(19) United States
(12) Patent Application Publication
(10) Pub. No.: US 2019/0070172 A1
(43) Pub. Date: Mar. 7, 2019

(54) LYSINE-SPECIFIC HISTONE DEMETHYLASE AS A NOVEL THERAPEUTIC TARGET IN MYELOPROLIFERATIVE NEOPLASMS

(71) Applicant: Imago Biosciences, Inc., San Carlos, CA (US)

(72) Inventor: Hugh Y. RIENHOFF, Jr., San Carlos, CA (US)

(21) Appl. No.: 15/773,911

(22) PCT Filed: Nov. 7, 2016
(86) PCT No.: PCT/US16/60847

§ 371 Date: May 4, 2018

Related U.S. Application Data
(60) Provisional application No. 62/251,674, filed on Nov. 5, 2015.

(51) Int. Cl. A61K 31/496; A61P 35/00; G01N 33/50

(52) U.S. Cl. CPC A61K 31/496; G01N 33/5094; A61P 35/00

(57) ABSTRACT

Disclosed herein are methods for treating or preventing myeloproliferative neoplasms in a subject in need thereof, and for effecting specific clinically relevant endpoints, comprising administering a therapeutically effective amount of an LSD1 inhibitor.

This application claims the benefit of priority of U.S. Provisional Application No. 62/251,674, filed Nov. 5, 2015, the disclosure of which is hereby incorporated by reference as if written herein in its entirety.

Myeloproliferative neoplasms (MPN), a disease category that includes polycythemia vera (PV), essential thrombocytosis (ET) and myelofibrosis (MF), are a distinct family of hematopoietic disorders caused by somatic mutations acquired by a multipotent hematopoietic stem/progenitor cell resulting in abnormalities in hematologic disturbances in red cell, white cell, and platelet production as well as splenomegaly and constitutional symptoms. The MPNs share common mutations which constitutively alter the normal physiologic signals responsible for hematopoiesis. MPN may present clinically as a benign clonal myeloproliferation but the initiating abnormal stem/progenitor cell is susceptible to new mutations and epigenetic alterations that allow for the rapid evolution to bone marrow failure with myelofibrosis or transformation to acute myelogenous leukemia (AML).

Many MPN patients are asymptomatic at the time of diagnosis. Confounding a definitive diagnosis and prognosis, ET, PV and PMF can masquerade as one another. Common presenting manifestations include fatigue, weight loss, night sweats, fever, dyspnea, and abdominal discomfort due to sometimes massive splenomegaly. The three MPN disorders overlap phenotypically and even share similarities with other myeloid neoplasms.

A specific point mutation in JAK2 (JAK2^{V617F}) as well as mutations in calreticulin (CALR) and the thrombopoietin receptor (MPL) are found in 90% of MPN patients. Although the distribution of these mutations is not equal among PV, ET and primary MF PMF), they do not diagnostically define the specific MPN or the prognosis nor are they mutually exclusive. Healthy individuals may carry one of these mutations without developing an MPN, indeed some of these mutations can be carried as germline mutations giving rise to hereditary forms of MPNs. ET, PV and PMF are nevertheless regarded as separate clinical entities each based on a distinct epidemiology, natural history and molecule profile. PV is the most common MPN and would appear to be the phenotypic manifestation mutations in JAK2. PV is the only MPN characterized by erythrocytosis defined as a hematocrit ≥60% and hemoglobin ≥20 gm/dL. ET is characterized by a sustained platelet count of >450,000/μL and occurs predominantly in women. MF, primary or secondary myelofibrosis but sometimes called myelofibrosis with myeloid metaplasia, agnogenic myeloid metaplasia, or primary myelosclerosis, is a chronic inflammatory process in which excess collagen is deposited in bone marrow impairing hematopoiesis in association with marrow fibrosis and extramedullary hematopoiesis.

The major complications arise from cytopenias secondary to bone marrow failure, extramedullary hematopoiesis, principally in the spleen and liver, and evolution to acute myeloid leukemia. To patients, splenomegaly is the most distressing complication of primary myelofibrosis, leading to mechanical discomfort, inanition, splenic infarction, portal and pulmonary hypertension and blood cell sequestration. Both ET and PV are complicated by thrombosis. ET and PV can progress to MF as well as to AML.

Many other somatic mutations found in MPN are also present in myelodysplastic syndrome (MDS) and de novo AML; these include mutations in DNMT3A, IDH1/2, TET2, ASXLI, EZH2, TP53, NF1, NRAS, KRAS, SF3BJ, U2AF1, SRSF2 and RUNX1. This shared mutational spectrum contributes to the phenotypic overlap of these disorders as well as influences their natural history including evolution to bone marrow failure or AML.

There is no treatment specific for primary myelofibrosis, essential thrombocythemia or polycythemia vera. Current treatments do not alter the natural history of disease significantly and are thus aimed principally at improving symptoms. Anemia associated with an erythropoietin (EPO) level <100 mU/ml may respond to recombinant EPO therapy but is associated with an increase in hepatosplenomegaly. Prednisone may be effective for patients with evidence of active inflammation or autoimmune disease. Hyperuricemia is managed with allopurinol. The nonselective JAK1/2 inhibitor ruxolitinib is approved for intermediate 1 and 2 and high risk MF patients and high-risk PV patients. Ruxolitinib is effective in alleviating constitutional symptoms and reducing spleen size or volume by 35% in approximately 50% of patients. Ruxolitinib prolonged survival and lowered the JAK^{V617F} allele burden in high-risk patients with primary MF (PMF). Anemia is exacerbated by ruxolitinib in some patients but thrombocytopenia, even if severe, may be improved. Ruxolitinib is effective only while the drug is administered; symptoms will recur when the drug is stopped. Fibrosis in the marrow is not affected and ruxolitinib has no impact on the mutation burden. Thalidomide at doses of 50 to 100 mg/day in combination with prednisone is effective in improving anemia and thrombocytopenia in approximately 60%, of primary myelofibrosis patients and reducing spleen size in approximately 20%. Interferon-α at low-doses to reduce splenomegaly can be effective in the early course of the illness but can cause cytopenias. Pegylated interferon can produce molecular remissions in PV and reverse myelofibrosis in PMF in a minority of patients. Hydroxycarbamide has a low incidence of acute toxicity but causes marrow suppression and is leukemogenic. Low-dose alkylating agents can reduce organomegaly, reverse marrow fibrosis, and improve blood counts but only occasionally has durable effects; alkylating agents can cause severe bone marrow suppression and are leukemogenic. The only potentially curative treatment is allogeneic bone marrow transplantation indicated for patients younger than 65 years of age with intermediate-2 or high DIPSS score who have a matched donor. Five-year survival from stem cell transplant averages is approximately 50%.

Epigenetic modifications of DNA such as methylation of cytosine or post-translational modifications of histones such as methylation and acetylation influence gene expression by altering chromatin structure. Changes in gene expression patterns have the potential to alter the phenotype of a given cell. Mutations in DNMT3A and TET2 are associated with changes in the normal methylation patterns of cytosine in DNA while mutations in JAK2, EZH2 and ASXLI alter the methylation, acetylation and phosphorylation state of histones: both of these classes of changes alter the patterns of normal gene expression programs. Mutations in genes coding for proteins influencing the epigenetic state of the cells suggest that targeting the enzymatic function of such proteins may selectively eliminate malignant stem/progenitor clones and/or restoring their normal phenotype.

Lysine-specific demethylase 1 (LSD1, also known as KDM1A) is an enzyme that removes mono- and dimethyl groups from histone (H) H3 at critical lysines (K), K4 and K9 (Shi et al., 2004). Methylation of histone H3K4 and H3K9 is a post-translational modification associated with changes in rates of gene transcription (Bannister and Kouzarides, 2011; Beisel and Paro, 2011). By virtue of altering the local state of chromatin, LSD1 is an epigenetic regulator of gene expression. The lysine (K) sites on histone H3 and the degree of methylation on those sites (1, 2 or 3 methyl groups) are associated with specific functions, e.g., enhancers and super-enhancers are characterized by H3K4me1 marks, whereas H3K4me2 is more often found in the proximal promoters and enhancers of actively transcribed genes (Campos and Reinberg, 2009; Gardner et al., 2011; Rando, 2012).

LSD1 is localized to three general regions of the genome: enhancers and super-enhancers, proximal promoters, and internal regions of transcription units through the agencies of proteins that bind DNA directly, generally TFs (Whyte et al., 2012; Whyte et al., 2013). Many TFs, both activators such as V-Myb Avian Myeloblastosis Viral Oncogene Homolog (MYB) and steroid hormone receptors, as well as repressors such as growth factor independence 1 transcription repressor (GFI1), recruit LSD1 to specific genomic locations (Metzger et al., 2005; Saleque et al., 2007; Lin et al., 2010). LSD1 is part of a larger protein complex, containing, e.g., Co-RE 1 silencing transcription factor (CoREST) or nucleosome remodeling and histone deacetylase (NuRD), which dictate the cell-specific chromatin remodeling (Lee et al., 2005; Foster et al., 2010). These complexes may also include DNMT1 and histone deacetylases 1, 2 and 3 (HDAC 1, 2, and 3) activities, all of which contribute to maintaining or modifying the epigenetic state at that genomic site (Shi et al., 2005; Orkin and Hochedlinger, 2011). Thus, an important property of LSD1 beyond its own enzymatic activity is its function as a scaffold for other epigenetic enzymes that are co-recruited to genomic sites. Among the many histone demethylases, LSD1 uniquely employs flavin adenine dinucleotide (FAD) to oxidatively remove one or two methyl groups in the process producing H2O2 and formaldehyde. As such, FAD is an essential co-factor for LSD1 activity (Shi et al., 2004). The other 33 histone lysine demethylases, the Jumonji types, employ an iron-dependent mechanism to remove methyl groups from histone lysines (Klose et al., 2006).

LSD1 is an essential gene; loss of LSD1 activity leads to early embryonic lethality (Wang et al., 2009; Foster et al., 2010). The protein is also needed for regulating the balance between self-renewal and proliferation (Wang et al., 2007). A conditional in vivo LSD1 knockdown (KD) using a doxycycline-inducible short hairpin LSD1 (shLSD1) established LSD1 as a central regulator of hematopoietic stem cells (HSCs) and myeloid progenitor cells (Sprussel et al., 2012). LSD1 KD resulted in profound but reversible thrombocytopenia, neutropenia and anemia; monocyte numbers were increased. LSD1 KD for 27 days led to an increase in circulating multipotent progenitors (MPPs) and HSCs with a concomitant down-regulation of chemokine (C-X-C motif) receptor 4 (CXCR4) without affecting the size of the dormant HSC pool (Sprussel et al., 2012). Impaired self-renewal was observed in long term HSCs 12 weeks following LSD1 excision using an inducible Cre system (M×1Cre mice×Lsd1fl/fl mice) (Kerenyi et al., 2013), consistent with LSD1 inhibition driving differentiation.

LSD1 plays a key role in regulating the progression from pluripotency to terminal differentiation (Adamo et al., 2011; Whyte et al., 2012). LSD1 is recruited to “high confidence” promoters and super-enhancers of genes essential for normal development by the “master” transcription factors octamer-binding transcription factor 4 (OCT4), SRY (sex determining region Y)-box 2 (SOX2), Nanog and the co-activator Mediator. Though not essential for maintenance of the embryonic stem cell (ESC) state, as part of the NuRD complex, LSD1 “decommissions” enhancers of genes directing the pluripotency program allowing ESC differentiation. LSD1 is essential for the complete shutdown of the ESC gene expression program as cells transition to more differentiated cell states (Whyte et al., 2012). The role LSD1 plays in the ESC is phenomenologically similar to the essential role LSD1 plays during myeloid hematopoiesis, in which enhancers active in HSCs generating a stem-cell gene expression signature are also “decommissioned”, allowing commitment of progenitors to specific myeloid lineages (Lara-Astiaso et al., 2014). Enhancers essential for terminal differentiation in lineage-specific progenitor cells are poised for activation by H3K4me1 marks while promoters are characterized by progressive methylation of H3K4 culminating in H3K4me3. Enhancer H3K27 acetylation locks in transcriptional activation and lineage commitment. Consistent with the need for stable H3K4 methylation during differentiation, LSD1 expression decreases dramatically as myeloid differentiation proceeds to terminal cell states (Lara-Astiaso et al., 2014). The LSD1 enzyme sits at the apex of myeloid hematopoiesis. LSD1 prevents myeloid differentiation in stem and myeloid progenitor cells but is down-regulated as cells commit to specific myeloid lineages (erythroid, granulocytic, and megakaryocytic). The inhibition of LSD1 in acute myeloid leukemia cells causes a loss of stem cell potential (clonogenicity) and a concomitant induction of differentiation to a more mature monocytic immunophenotype. In mouse models of myeloproliferative neoplasm, treatment with LSD1 inhibitors reduces the mutant progenitor cell population consistent with the role LSD1 plays in sustaining the self-renewal phenotype.

As a key factor in regulating myeloid maturation, LSD1 is suitable as a target for a variety of myeloproliferative neoplasms. There are three major myeloproliferative neoplasms that may be treated with an LSD1 inhibitor: polycythemia vera, essential thrombocythemia, primary myelofibrosis (or myelofibrosis secondary to PV and ET); other MPNs are disclosed below and may also be treated by the methods disclosed herein. Other MPNs include All begin as clonal disorders as a consequence of somatic mutations occurring in hematopoietic stem/progenitor cells. The clinical overlap among these related diseases is mirrored by their shared genetic spectrum of somatic mutations including mutations in JAK2, DNMT3A, MPL, CALR, and ASXLI. In mouse models of myelofibrosis (Jak2^{V617F} and Mpl^{W515L}), inhibition of LSD1 causes a significant improvement in five parameters of disease: reduction in platelets, reduction in splenomegaly, reduction in red cell count, resolution of marrow fibrosis and reduction in mutant cell burden.

Among BCR-ABL-negative myeloproliferative neoplasms, primary myelofibrosis and post-PV/ET myelofibrosis (PPV-MF and PET-MF) are associated with the highest degree of morbidity and mortality, including progressive bone marrow (BM) fibrosis and resultant BM failure. Although the JAK inhibitor ruxolitinib is now approved for the treatment of MF-associated splenomegaly and systemic symptoms, JAK inhibitor therapy does not reduce the population of JAK2-mutant cells in MPN patients. The limited ability of JAK inhibition to induce clinically meaningful molecular responses in MPN patients underscores the need for the development of more effective therapies for these JAK kinase/STAT-dependent malignancies.

Recent studies have shown that the lysine-specific histone demethylase, LSD1 (KDM1A), participates in the balance in hematopoietic stem/progenitor cells between proliferation and differentiation in vivo by influencing state-specific gene expression patterns. In physiologic hematopoiesis, LSD1 is essential for normal myeloid differentiation affecting the erythroid, megakaryocytic and granulocytic lineages but not the monocytic/dendritic lineage. Small molecule inhibitors of LSD1 have shown promising results in preclinical models of acute myeloid leukemia (AML) and solid cancers and have recently entered clinical trials in AML. However, the role and requirement for LSD1 in the pathogenesis of MPNs and the therapeutic targeting of LSD1 in MPN is an area of current investigation.

WO 2012/107498 discloses the use of certain LSD1 inhibitors to treat the Philadelphia chromosome negative myeloproliferative disorders essential thrombocythemia, myelofibrosis, and polycythemia vera. There remains a need for potent LSD1 inhibitors with demonstrated ability to treat myelofibrosis and other myeloproliferative neoplasms, and attendant symptoms.

### DETAILED DESCRIPTION OF THE INVENTION

Provided herein is a method for treating or preventing a myeloproliferative neoplasm in a subject in need thereof, the method comprising administering a therapeutically effective amount of an LSD1 inhibitor.

Further provided is a method for suppressing proliferation of malignant myeloid cells in a subject in need thereof, the method comprising administering a therapeutically effective amount of an LSD1 inhibitor.

Also provided is a method for reducing reticulin and collagen bone marrow fibrosis in a subject in need thereof, the method comprising administering a therapeutically effective amount of an LSD1 inhibitor.

Also provided is a method for reducing plasma levels of one or more inflammatory cytokines in a subject in need thereof, the method comprising administering a therapeutically effective amount of an LSD1 inhibitor.

Also provided is a method for reducing the mass of malignant myeloid cells in a subject in need thereof, the method comprising administering a therapeutically effective amount of an LSD1 inhibitor.

Also provided is a method for reducing abnormal spleen size or volume in a subject in need thereof, the method comprising administering a therapeutically effective amount of an LSD1 inhibitor.

Also provided is a method for reducing the amount of extramedullary hematopoiesis in a subject in need thereof, the method comprising administering a therapeutically effective amount of an LSD1 inhibitor.

Also provided is a method for reducing the constitutional symptoms of myelofibrosis measured by patient-reported surveys in a subject in need thereof, the method comprising administering a therapeutically effective amount of an LSD1 inhibitor.

Also provided is a method for reducing platelet counts in a subject in need thereof, the method comprising administering a therapeutically effective amount of an LSD1 inhibitor.

Also provided is a method for reducing bone marrow cellularity to age-adjusted normocellularity with fewer than 5% blast cells in a subject in need thereof, the method comprising administering a therapeutically effective amount of an LSD1 inhibitor.

Also provided is a method for a) reducing hemoglobin level in a PV patient to <160 g/L, or b) decreasing red cell mass in a PV patient, wherein the decrease is inferred from hemoglobin levels Hb of <160 g/L, either comprising administering a therapeutically effective amount of an LSD1 inhibitor. Also provided is a method for increasing hemoglobin to >100 g/L in a MF patient, comprising administering a therapeutically effective amount of an LSD inhibitor. Also provided is a method for increasing hemoglobin to a value >100 g/L and less than the upper limit of age- and sex adjusted normal in a subject in need thereof, the method comprising administering a therapeutically effective amount of an LSD1 inhibitor.

In certain embodiments of each of the above methods, the LSD1 inhibitor is a compound of Formula I:

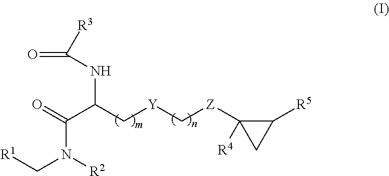

or a salt thereof, wherein:

Y is chosen from a bond, NR^{4a}, O, C(O)NH, NHC(O), S, SO₂, and CH₂;

Z is chosen from a bond, NR^{4b}, O, C(O)NH, NHC(O), S, SO₂, and CH₂;

m is an integer from 0 to 5;

n is an integer from 0 to 3;

R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;

R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;

R⁴, R^{4a}, and R^{4b} are independently chosen from hydrogen, alkyl, alkenyl, alkynyl, and cycloalkyl;

R⁵ is chosen from aryl and heteroaryl, any of which may be optionally substituted with between 0 and 3 R⁶ groups;

each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and

R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;

optionally with the proviso that when Y═CH₂, R⁴═H, and Z═R^{4b}, then m+n≠3.

In certain embodiments of each of the above methods, the LSD1 inhibitor is a compound of Formula II:

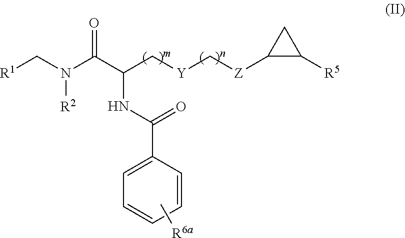

or a salt, polymorph, or solvate thereof, wherein:

Y is chosen from a bond, NR^{4a}, O, C(O)NH, NHC(O), S, SO₂, CHOH, and CH₂;

Z is chosen from a bond, NR^{4b}, O, C(O)NH, NHC(O), S, SO₂, and CH₂;

m is chosen from 0, 1, 2, 3, 4, and 5;

n is chosen from 0, 1, 2, and 3;

R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;

R^{4a} and R^{4b} are independently chosen from hydrogen, alkyl, alkenyl, alkynyl, and cycloalkyl;

R⁵ is chosen from aryl and heteroaryl, any of which may be optionally substituted with between 0 and 3 R⁶ groups;

R^{6a} is chosen from heteroaryl, cyano, and S(O)₂N(CH₃)₂;

each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and

R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;

optionally with the proviso that when Y═CH₂ and Z═R^{4b}, then m+n≠3.

In certain embodiments of each of the above methods, the LSD1 inhibitor is a compound as disclosed below, of any of Formulas III, IV or IV, or an embodiment thereof, or any one of Examples A1-A34, Examples 1-188, and the Examples disclosed in Paragraph [0191] of the specification, or a compound as disclosed in any of the references listed in paragraph [0176] below.

### BRIEF DESCRIPTION OF THE DRAWINGS

FIG. 1 presents data with Compound 1 in the MPLW515L-driven ET/MF mouse model. FIGS. 1a and 1b show, respectively, white blood cell counts and platelet counts in animals receiving either Compound 1 or vehicle. Animals receiving Compound 1 showed significant reduction of white blood cell count and platelets compared to vehicle-treated animals. FIG. 1c shows levels of the serum levels of the cytokine Cxcl5 in the two groups of animals; drug-treated animals had significantly lower levels of Cxcl5. FIG. 1d shows the levels of GFP-tagged mutant gene in drug-treated and vehicle-treated animals. Animals receiving Compound 1 showed significant reduction of mutant allele burden. FIG. 1e, shows hematoxylin and eosin (H+E) staining of reticulin and collagen of animals receiving either Compound 1 or vehicle. Compound 1-treated animals had lower levels of H+E staining compared to vehicle-only animals, indicated reduced bone marrow fibrosis.

FIG. 2a shows data with Compound 2 bis-tosylate in the JAK2^{V617F} knock-in mouse model of myeloproliferative neoplasm. FIG. 2a shows platelet counts in animals receiving Compound 2 bis-tosylate or vehicle. Animals receiving Compound 2 bis-tosylate showed markedly reduced platelet levels as compared to vehicle controls.

FIG. 2b shows white blood cell counts in animals receiving Compound 2 bis-tosylate or vehicle. Animals receiving Compound 2 bis-tosylate showed markedly reduced white blood cell counts as compared to vehicle controls.

FIG. 2c shows hematocrit in animals receiving Compound 2 bis-tosylate or vehicle. Animals receiving Compound 2 bis-tosylate showed a reduction in hematocrit as compared to vehicle controls.

### ABBREVIATIONS AND DEFINITIONS

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

A “therapeutically effective amount” of a drug is an amount of drug or its pharmaceutically acceptable salt that eliminates, alleviates, or provides relief of the symptoms of the disease for which it is administered.

A “subject in need thereof” is a human or non-human animal that exhibits one or more symptoms or indicia of a disease.

When ranges of values are disclosed, and the notation “from n₁, . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonylalkyl include, but are not limited to, methylsulfonylmethyl and ethylsulfonylmethyl.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched-chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, hydroxyalkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “amino acid”, as used herein, alone or in combination, refers to a —NHCHRC(O)O— group, which may be attached to the parent molecular moiety to give either an N-terminus or C-terminus amino acid, wherein R is independently chosen from hydrogen, alkyl, aryl, heteroaryl, heterocycloalkyl, aminoalkyl, amido, amidoalkyl, carboxyl, carboxylalkyl, guanidinealkyl, hydroxyl, thiol, and thioalkyl, any of which themselves may be optionally substituted. The term C-terminus, as used herein, alone or in combination, refers to the parent molecular moiety being bound to the amino acid at the amino group, to give an amide as described herein, with the carboxyl group unbound, resulting in a terminal carboxyl group, or the corresponding carboxylate anion. The term N-terminus, as used herein, alone or in combination, refers to the parent molecular moiety being bound to the amino acid at the carboxyl group, to give an ester as described herein, with the amino group unbound resulting in a terminal secondary amine, or the corresponding ammonium cation. In other words, C-terminus refers to —NHCHRC(O)OH or to —NHCHRC(O)O⁻ and N-terminus refers to H₂NCHRC(O)O— or to H₃N⁺CHRC(O)O—.

The term “aryl”, as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The term azetidine, as used herein, alone or in combination, refers to an

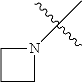 [SMILES: *N1CCC1]

group.

The term pyrrolidine, as used herein, alone or in combination, refers to a

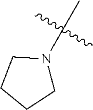 [SMILES: *N1CCCC1]

group.

The term imidazolidine, as used herein, alone or in combination, refers to a

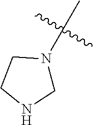 [SMILES: *N1CCNC1]

group.

The term pyrazolidine, as used herein, alone or in combination, refers to a

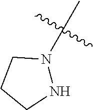 [SMILES: *N1CCCN1]

group.

The term thiomorpholine, as used herein, alone or in combination, refers to a

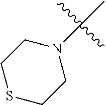 [SMILES: *N1CCSCC1]

group.

The term pyrrole, as used herein, alone or in combination, refers to a

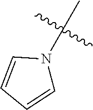 [SMILES: *n1cccc1]

group.

The term pyrazole, as used herein, alone or in combination, refers to a N

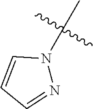 [SMILES: *n1cccn1]

group.

The terms “benzo” and “benz,” as used herein, alone or in combination, refer to the divalent group C₆H₄═ derived from benzene. Examples include benzothiophene and benzimidazole.

The term “biphenyl” as used herein refers to two phenyl groups connected at one carbon site on each ring.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′ group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “guanidine”, as used herein, alone or in combination, refers to —NHC(═NH)NH₂, or the corresponding guanidinium cation.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogen atoms are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms chosen from O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom chosen from O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furanyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl, azepinyl, diazepinyl, benzazepinyl, and the like. Exemplary tricyclic heterocyclic groups include carbazolyl, benzidolyl, phenanthrolinyl, dibenzofuranyl, acridinyl, phenanthridinyl, xanthenyl and the like.

The term “heteroarylalkyl” as used herein alone or as part of another group refers to alkyl groups as defined above having a heteroaryl substituent.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, imidazolidinyl, isoindolinyl, morpholinyl, oxazolidinyl, isoxazolidinyl, piperidinyl, piperazinyl, methylpiperazinyl, N-methylpiperazinyl, pyrrolidinyl, pyrazolidinyl, tetrahydrofuranyl, tetrahydropyridinyl, thiomorpholinyl, thiazolidinyl, diazepanyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “hydroxamic acid”, as used herein, alone or in combination, refers to —C(═O)NHOH, wherein the parent molecular moiety is attached to the hydroxamic acid group by means of the carbon atom.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either 1) monocyclic heteroaryl comprising five or six ring members, of which between one and four said members may be heteroatoms chosen from O, S, and N, or 2) bicyclic heteroaryl, wherein each of the fused rings comprises five or six ring members, comprising between them one to four heteroatoms chosen from O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphonate,” as used herein, alone or in combination, refers to a —P(═O)(OR)₂ group, wherein R is chosen from alkyl and aryl. The term “phosphonic acid”, as used herein, alone or in combination, refers to a —P(═O)(OH)₂ group.

The term “phosphoramide”, as used herein, alone or in combination, refers to a —P(═O)(NR)₃ group, with R as defined herein.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethoxy” refers to a X₃CO— group where X is a halogen.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. Similarly, when a designation such as “n” which may be chosen from a group or range of integers is designated to be 0, then the group which it designates is either absent, if in a terminal position, or condenses to form a bond, if it falls between two other groups.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reaction of the appropriate compound, in the form of the free base, with the appropriate acid.

The compounds disclosed herein can exist as polymorphs and other distinct solid forms such as solvates, hydrates, and the like. A compound may be a polymorph, solvate, or hydrate of a salt or of the free base or acid.

The term “myeloproliferative neoplasm” (MPN) refers to blood cancers that occur when the body makes too many white or red blood cells, or platelets as a consequence of somatic mutations that activate the hormone signaling pathways that control the production of these types of blood cells. They are “clonal diseases of hematopoietic stem cells” given that the neoplastic cells arise from a single mutant clone arising from bone marrow cells (Campregher et al. Rev Bras Hematol Hemoter. 2012; 34(2): 150-5). MPNs include polycythemia vera (PV), myelofibrosis including primary myelofibrosis (PMF, including, in certain embodiments, both the prefibrotic/early stage and the overt fibrotic stage) and post-PV/ET myelofibrosis (PPV-MF and PET-MF), essential thrombocythemia (ET), chronic neutrophilic leukemia (CNL), chronic eosinophilic leukemia, not otherwise specified (CEL-NOS), and chronic myeloid leukemia (CML), as well as other unclassifiable MPNs. For a more thorough discussion of MPNs and related myeloid neoplasms and acute leukemia, as well as diagnostic criteria for PV, ET, PMF, and other MPNs, see Arber et al. “The 2016 revision to the World Health Organization classification of myeloid neoplasms and acute leukemia”, Blood 2016, 127(20):2391-2405. For a thorough discussion of myelofibrosis diagnostic and response criteria, see Tefferi A et al., “Revised response criteria for myelofibrosis: International Working Group-Myeloproliferative Neoplasms Research and Treatment (IWG-MRT) and European LeukemiaNet (ELN) consensus report,” Blood, 122(8):1395-98 (2013).

### Formulations

While it may be possible for the compounds disclosed herein to be administered as the raw chemical, it is also possible to present them as pharmaceutical formulations (equivalently, “pharmaceutical compositions”). Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, intraadiposal, intraarterial, intracranial, intralesional, intranasal, intraocular, intrapericardial, intraperitoneal, intrapleural, intraprostatical, intrarectal, intrathecal, intratracheal, intratumoral, intraumbilical, intravaginal, intravesicular, intravitreal, and intramedullary), intraperitoneal, rectal, topical (including, without limitation, dermal, buccal, sublingual, vaginal, rectal, nasal, otic, and ocular), local, mucosal, sublingual, subcutaneous, transmucosal, transdermal, transbuccal, transdermal, and vaginal; liposomal, in cremes, in lipid compositions, via a catheter, via a lavage, via continuous infusion, via infusion, via inhalation, via injection, via local delivery, via localized perfusion, bathing target cells directly, or any combination thereof. Administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as hard or soft capsules, wafers, cachets or tablets each containing a predetermined amount of the active ingredient; as a powder or granules; as a syrup, elixir, solution, or a suspension in an aqueous liquid or a non-aqueous liquid; or as an oil-in-water liquid emulsion, a water-in-oil liquid emulsion, or a compound dispersed in a liposome. The active ingredient may also be presented as a bolus, electuary or paste.

Pharmaceutical preparations that can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide delayed, slowed, or controlled release or absorption of the active ingredient therein. Compositions may further comprise an agent that enhances solubility or dispersability. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Depending on the route of administration, the compounds, or granules or particles thereof, may be coated in a material to protect the compounds from the action of acids and other natural conditions that may inactivate the compounds.

The compounds may be formulated for parenteral administration by injection, e.g., by bolus injection or continuous infusion, either to the body or to the site of a disease or wound. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. To administer the therapeutic compound by other than parenteral administration, it may be necessary to coat the compound with, or co-administer the compound with a material to prevent its inactivation (for example, via liposomal formulation).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Topical ophthalmic, otic, and nasal formulations disclosed herein may comprise excipients in addition to the active ingredient. Excipients commonly used in such formulations include, but are not limited to, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants. Any of a variety of excipients may be used in formulations disclosed herein including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, guar gum, xanthan gum, carrageenan, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the active ingredient. In preferred embodiments, the excipients to be included in the formulations are typically selected because of their inertness towards the active ingredient component of the formulations.

Relative to ophthalmic, otic, and nasal formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants (though nonionic surfactants are preferred), RLM 100, POE 20 cetylstearyl ethers such as Procol® CS20 and poloxamers such as Pluronic® F68.

The formulations set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, amino alcohols such as AMP-95, or sorbic acid. In certain embodiments, the formulation may be self-preserved so that no preservation agent is required.

In certain topical embodiments, formulations are prepared using a buffering system that maintains the formulation at a pH of about 4.5 to a pH of about 8. In further embodiments, the pH is from 7 to 8.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. Several optional ingredients can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, galactomannan polymers (such as guar and derivatives thereof), and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage, an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example, a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

The therapeutic compound may also be administered intraspinally or intracerebrally. Dispersions for these types of administrations can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and required other ingredients to be pharmacologically sound. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

It should be understood that in addition to the ingredients particularly mentioned above, the formulations described above may include other agents conventional in the art having regard to the type of formulation in question, for example, those suitable for oral administration may include flavoring agents.

Compounds may be administered at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

Preferred unit dosage formulations are those containing an effective dose, as herein below recited, or an appropriate fraction thereof, of the active ingredient. In certain embodiments, a formulation disclosed herein is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

The amount of active ingredient that may be combined with the carrier materials to produce a single dosage form will vary depending upon the host treated and the particular mode of administration. Similarly, the precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Alternatively, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). There is even the possibility that two compounds, one of the compounds described herein and a second compound may together achieve the desired therapeutic effect that neither alone could achieve. Alternatively, by way of example only, the benefit experienced by a patient may be increased by administering one of the compounds described herein with another therapeutic agent (which also includes a therapeutic regimen) that also has therapeutic benefit. By way of example only, in a treatment for acute myelogenous leukemia or sickle cell anemia involving administration of one of the compounds described herein, increased therapeutic benefit may result by also providing the patient with another therapeutic agent for sickle cell anemia or for acute myelogenous leukemia. In any case, regardless of the disease, disorder or condition being treated, the overall benefit experienced by the patient may simply be additive of the two therapeutic agents or the two agents may have synergistic therapeutic effects in a patient.

Effective combination therapy may be achieved with a single composition or pharmacological formulation that includes both agents, or with two distinct compositions or formulations, at the same time, wherein one composition includes a compound of the present disclosure, and the other includes the second agent(s). Alternatively, the therapy may precede or follow the other agent treatment by intervals ranging from minutes to months. Administration of the compounds of the present disclosure to a patient will follow general protocols for the administration of pharmaceuticals, taking into account the toxicity, if any, of the drug. It is expected that the treatment cycles would be repeated as necessary.

Specific, non-limiting examples of possible combination therapies include use of certain compounds of the invention with the following agents and classes of agents: agents that inhibit DNA methyltransferases such as decitabine or 5′-aza-cytadine; agents that inhibit the activity of histone deacetylases, histone de-sumoylases, histone de-ubiquitinases, or histone phosphatases such as hydroxyurea; antisense RNAs that might inhibit the expression of other components of the protein complex bound at the DR site in the gamma globin promoter; agents that inhibit the action of Klf1 or the expression of KLF1; agents that inhibit the action of Bcl11a or the expression of BCL11A; and agents that inhibit cell cycle progression such as hydroxyurea, ara-C or daunorubicin; agents that induce differentiation in leukemic cells such as all-trans retinoic acid (ATRA).

Thus, in another aspect, the present invention provides methods for treating diseases or disorders in a human or animal subject in need of such treatment comprising administering to said subject an amount of a compound disclosed herein effective to reduce or prevent said disorder in the subject in combination with at least one additional agent for the treatment of said disorder that is known in the art.

### Compounds

Compounds useful in the methods disclosed herein include those disclosed in the following references:

| | | |
|---|---|---|
| WO2014/164867A1 | WO2012/013727A1 | US2015/0225394A1 |
| WO2015/021128A1 | WO2012/042042A1 | US2015/0225401A1 |
| WO2016/130952A1 | WO2012/045883A1 | US2015/0225375A1 |
| U.S. Pat. No. 9,388,123 | WO2012/072713A2 | US2015/0225379A1 |
| WO2010/143582A1 | WO2012/107499A1 | US2016/0009711A1 |
| WO2012/135113A2 | WO2012/107498A1 | US2016/0009712A1 |
| WO2009/027349A2 | WO2012/156537A2 | US2016/0009720A1 |
| WO2010/043721A1 | WO2012/156531A2 | US2016/0009721A1 |
| WO2010/084160A1 | WO2013/057320A1 | WO2016/161282A1 |
| WO2011/035941A1 | WO2013/057322A1 | US2015/0315126A1 |
| WO2011/042217A1 | U.S. Pat. No. 8,765,820 | US2015/0065495A1 |
| WO2011/106105A2 | U.S. Pat. No. 8,389,580 | WO2015/120281A1 |
| WO2011/106106A2 | US2016/0120862A1 | WO2016/123387A1 |
| WO2011/106573A2 | US2015/0025054A1 | WO2016172496A1 |
| WO2011/106574A2 | WO2015/134973A1 | US2015/0065495A1 |
| WO2011/131697A1 | US2014/0011857A1 | |
| WO2012/013728A1 | US2012/0322877A1 | |

the contents of which are hereby incorporated by reference as if written herein in their entireties.

Examples of LSD1-inhibiting compounds which may be used in the methods disclosed herein include the compounds in Table I below.

TABLE 1
Compound Examples

| Ex | |
|---|---|
| # | Structure |
| A1 | 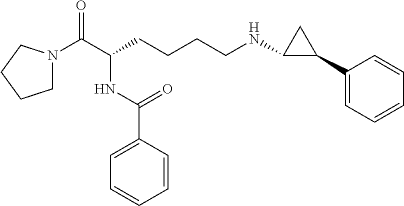 [SMILES: O=C(N[C@@H](CCCCN[C@@H]1C[C@H]1c1ccccc1)C(=O)N1CCCC1)c1ccccc1] |
| A2 | 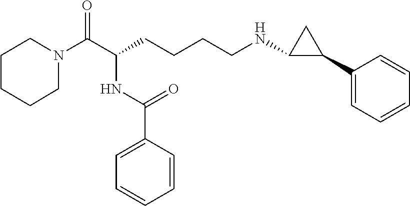 [SMILES: O=C(N[C@@H](CCCCN[C@@H]1C[C@H]1c1ccccc1)C(=O)N1CCCCC1)c1ccccc1] |
| A3 Cpd1 | 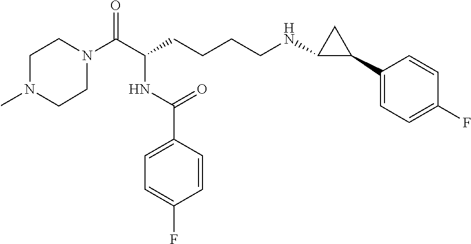 [SMILES: CN1CCN(C(=O)[C@H](CCCCN[C@@H]2C[C@H]2c2ccc(F)cc2)NC(=O)c2ccc(F)cc2)CC1] |
| A4 | 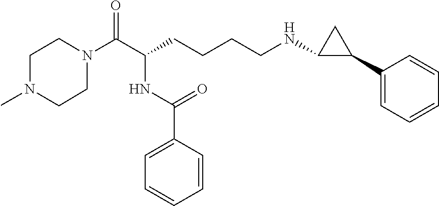 [SMILES: CN1CCN(C(=O)[C@H](CCCCN[C@@H]2C[C@H]2c2ccccc2)NC(=O)c2ccccc2)CC1] |
| A5 | 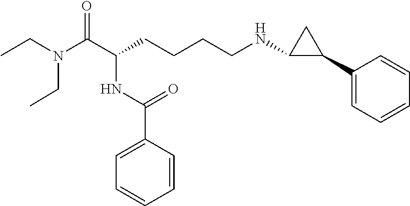 [SMILES: CCN(CC)C(=O)[C@H](CCCCN[C@@H]1C[C@H]1c1ccccc1)NC(=O)c1ccccc1] |
| A6 | 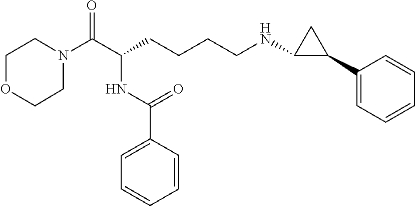 [SMILES: O=C(N[C@@H](CCCCN[C@@H]1C[C@H]1c1ccccc1)C(=O)N1CCOCC1)c1ccccc1] |
| A7 | 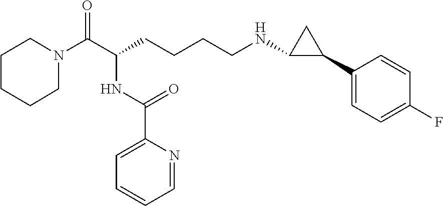 [SMILES: O=C(N[C@@H](CCCCN[C@@H]1C[C@H]1c1ccc(F)cc1)C(=O)N1CCCCC1)c1ccccn1] |
| A8 | 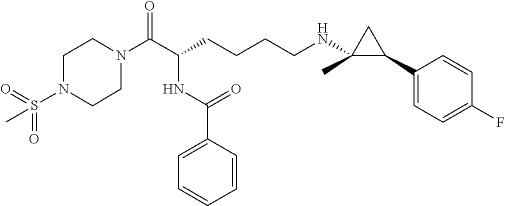 [SMILES: C[C@@]1(NCCCC[C@H](NC(=O)c2ccccc2)C(=O)N2CCN(S(C)(=O)=O)CC2)C[C@H]1c1ccc(F)cc1] |
| A9 | 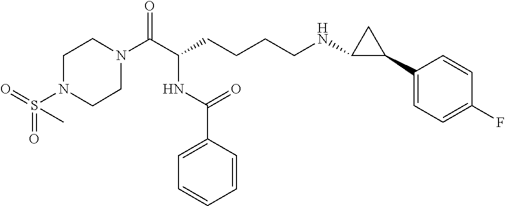 [SMILES: CS(=O)(=O)N1CCN(C(=O)[C@H](CCCCN[C@@H]2C[C@H]2c2ccc(F)cc2)NC(=O)c2ccccc2)CC1] |
| A10 | 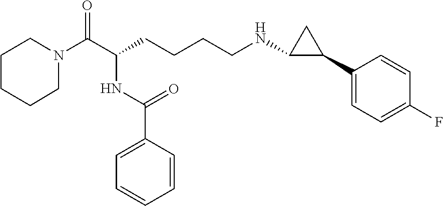 [SMILES: O=C(N[C@@H](CCCCN[C@@H]1C[C@H]1c1ccc(F)cc1)C(=O)N1CCCCC1)c1ccccc1] |
| A11 | 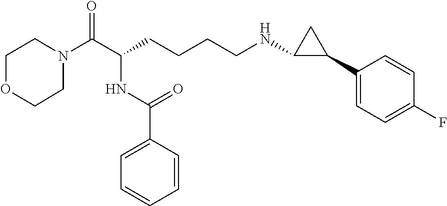 [SMILES: O=C(N[C@@H](CCCCN[C@@H]1C[C@H]1c1ccc(F)cc1)C(=O)N1CCOCC1)c1ccccc1] |
| A12 | 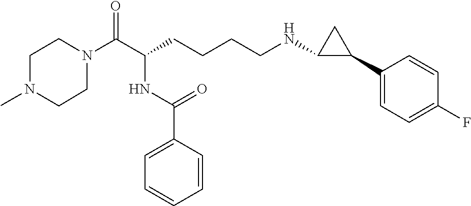 [SMILES: CN1CCN(C(=O)[C@H](CCCCN[C@@H]2C[C@H]2c2ccc(F)cc2)NC(=O)c2ccccc2)CC1] |
| A13 | 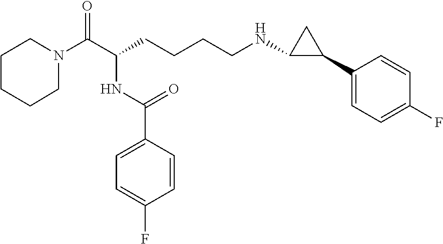 [SMILES: O=C(N[C@@H](CCCCN[C@@H]1C[C@H]1c1ccc(F)cc1)C(=O)N1CCCCC1)c1ccc(F)cc1] |
| A14 | 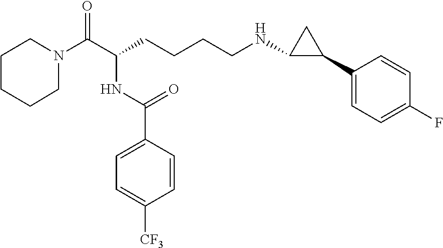 [SMILES: O=C(N[C@@H](CCCCN[C@@H]1C[C@H]1c1ccc(F)cc1)C(=O)N1CCCCC1)c1ccc(C(F)(F)F)cc1] |
| A15 | 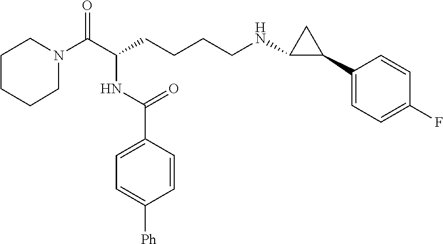 [SMILES: O=C(N[C@@H](CCCCN[C@@H]1C[C@H]1c1ccc(F)cc1)C(=O)N1CCCCC1)c1ccc(-c2ccccc2)cc1] |
| A16 | 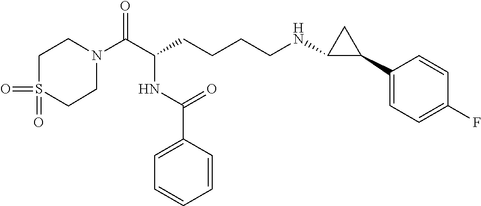 [SMILES: O=C(N[C@@H](CCCCN[C@@H]1C[C@H]1c1ccc(F)cc1)C(=O)N1CCS(=O)(=O)CC1)c1ccccc1] |
| A17 | 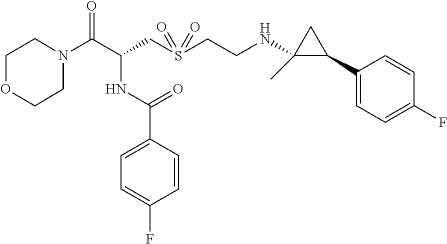 [SMILES: C[C@@]1(NCCS(=O)(=O)C[C@H](NC(=O)c2ccc(F)cc2)C(=O)N2CCOCC2)C[C@H]1c1ccc(F)cc1] |
| A18 | 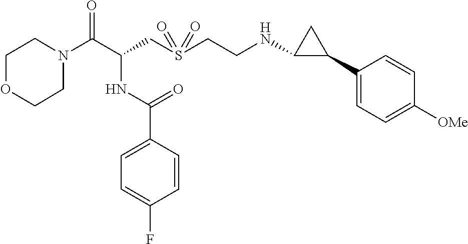 [SMILES: COc1ccc([C@@H]2C[C@H]2NCCS(=O)(=O)C[C@H](NC(=O)c2ccc(F)cc2)C(=O)N2CCOCC2)cc1] |
| A19 | 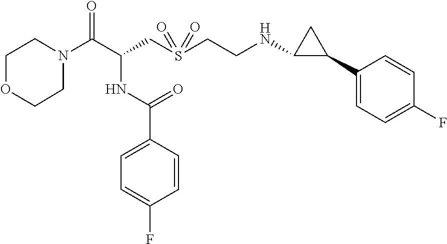 [SMILES: O=C(N[C@@H](CS(=O)(=O)CCN[C@@H]1C[C@H]1c1ccc(F)cc1)C(=O)N1CCOCC1)c1ccc(F)cc1] |
| A20 | 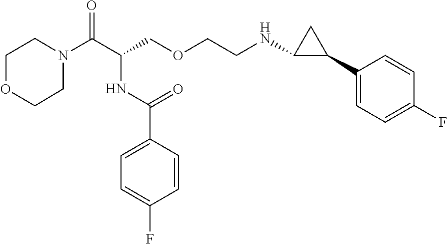 [SMILES: O=C(N[C@@H](COCCN[C@@H]1C[C@H]1c1ccc(F)cc1)C(=O)N1CCOCC1)c1ccc(F)cc1] |
| A21 | 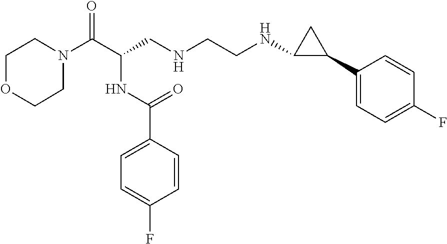 [SMILES: O=C(N[C@@H](CNCCN[C@@H]1C[C@H]1c1ccc(F)cc1)C(=O)N1CCOCC1)c1ccc(F)cc1] |
| A22 | 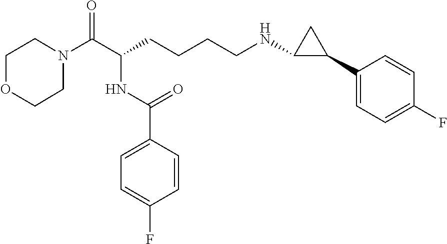 [SMILES: O=C(N[C@@H](CCCCN[C@@H]1C[C@H]1c1ccc(F)cc1)C(=O)N1CCOCC1)c1ccc(F)cc1] |
| A23 | 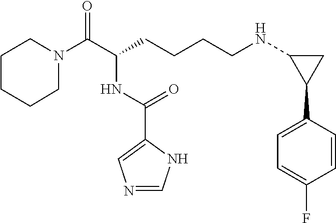 |
| A24 | 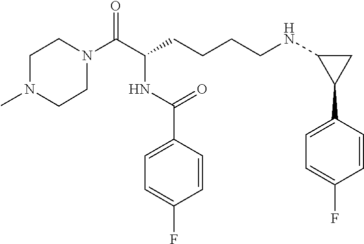 |
| A25 | 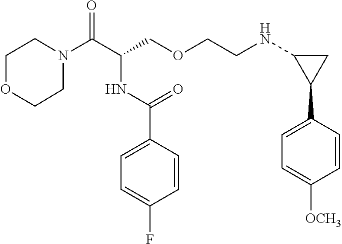 |
| A26 | 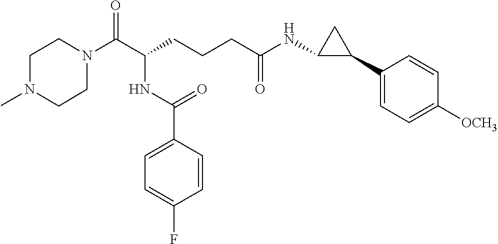 [SMILES: COc1ccc([C@@H]2C[C@H]2NC(=O)CCC[C@H](NC(=O)c2ccc(F)cc2)C(=O)N2CCN(C)CC2)cc1] |
| A27 | 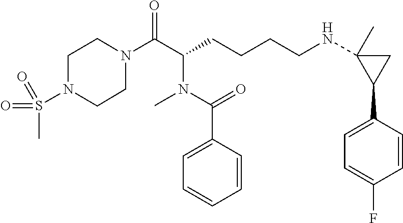 |
| A28 | 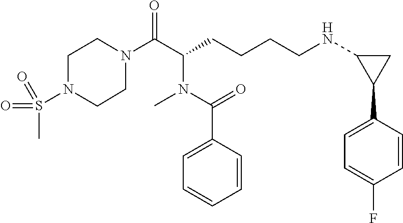 |
| A29 | 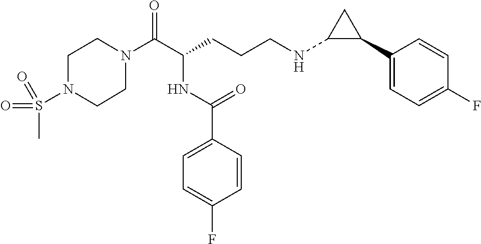 |
| A30 | 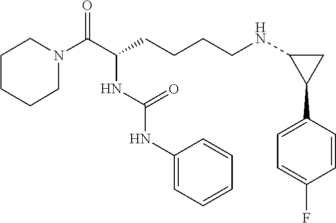 |
| A31 | 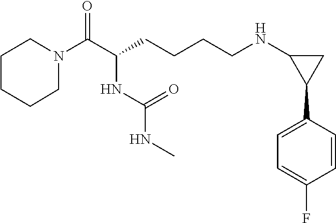 [SMILES: CNC(=O)N[C@@H](CCCCNC1C[C@H]1c1ccc(F)cc1)C(=O)N1CCCCC1] |
| A32 | 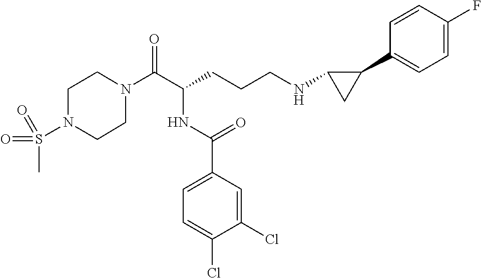 [SMILES: CS(=O)(=O)N1CCN(C(=O)[C@H](CCCN[C@H]2C[C@@H]2c2ccc(F)cc2)NC(=O)c2ccc(Cl)c(Cl)c2)CC1] |
| A33 | 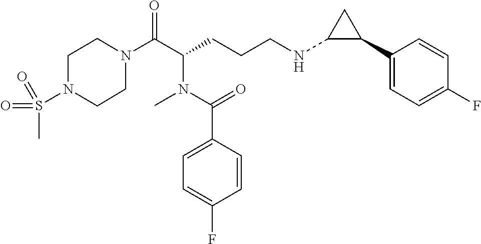 |
| A34 | 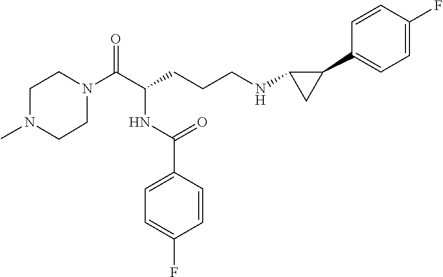 [SMILES: CN1CCN(C(=O)[C@H](CCCN[C@H]2C[C@@H]2c2ccc(F)cc2)NC(=O)c2ccc(F)cc2)CC1] |
| 1 | 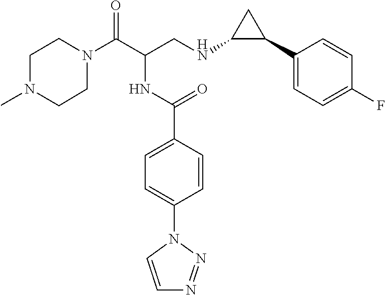 [SMILES: CN1CCN(C(=O)C(CN[C@@H]2C[C@H]2c2ccc(F)cc2)NC(=O)c2ccc(-n3ccnn3)cc2)CC1] |
| 2 | 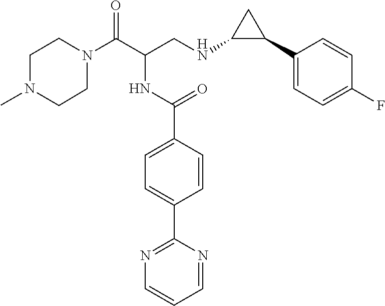 [SMILES: CN1CCN(C(=O)C(CN[C@@H]2C[C@H]2c2ccc(F)cc2)NC(=O)c2ccc(-c3ncccn3)cc2)CC1] |
| 3 | 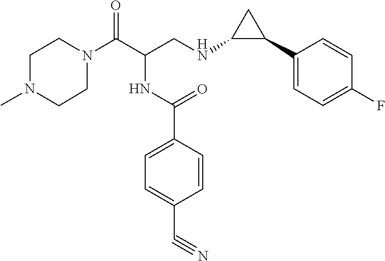 [SMILES: CN1CCN(C(=O)C(CN[C@@H]2C[C@H]2c2ccc(F)cc2)NC(=O)c2ccc(C#N)cc2)CC1] |
| 4 | 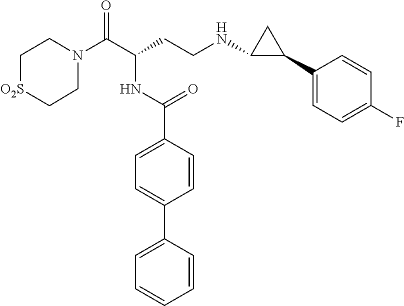 [SMILES: O=C(N[C@@H](CCN[C@@H]1C[C@H]1c1ccc(F)cc1)C(=O)N1CCS(=O)(=O)CC1)c1ccc(-c2ccccc2)cc1] |
| 5 | 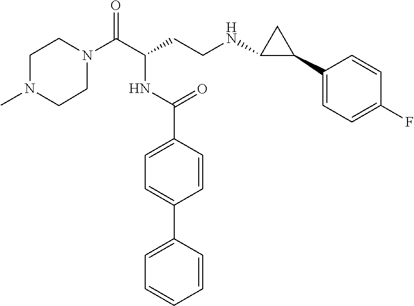 [SMILES: CN1CCN(C(=O)[C@H](CCN[C@@H]2C[C@H]2c2ccc(F)cc2)NC(=O)c2ccc(-c3ccccc3)cc2)CC1] |
| 6 | 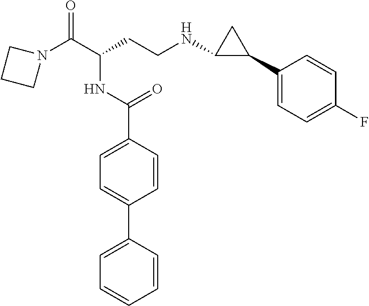 [SMILES: O=C(N[C@@H](CCN[C@@H]1C[C@H]1c1ccc(F)cc1)C(=O)N1CCC1)c1ccc(-c2ccccc2)cc1] |
| 7 | 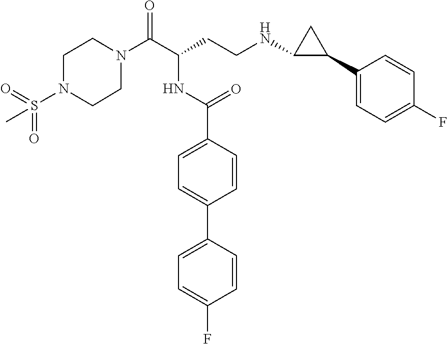 [SMILES: CS(=O)(=O)N1CCN(C(=O)[C@H](CCN[C@@H]2C[C@H]2c2ccc(F)cc2)NC(=O)c2ccc(-c3ccc(F)cc3)cc2)CC1] |
| 8 | 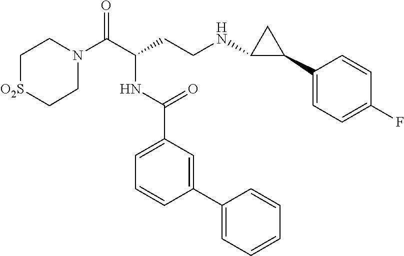 [SMILES: O=C(N[C@@H](CCN[C@@H]1C[C@H]1c1ccc(F)cc1)C(=O)N1CCS(=O)(=O)CC1)c1cccc(-c2ccccc2)c1] |
| 9 | 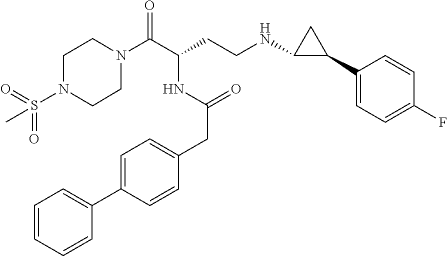 [SMILES: CS(=O)(=O)N1CCN(C(=O)[C@H](CCN[C@@H]2C[C@H]2c2ccc(F)cc2)NC(=O)Cc2ccc(-c3ccccc3)cc2)CC1] |
| 10 | 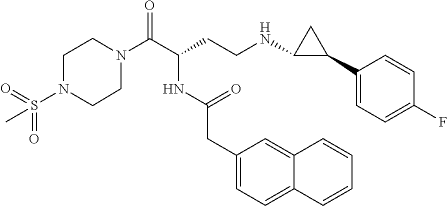 [SMILES: CS(=O)(=O)N1CCN(C(=O)[C@H](CCN[C@@H]2C[C@H]2c2ccc(F)cc2)NC(=O)Cc2ccc3ccccc3c2)CC1] |
| 11 | 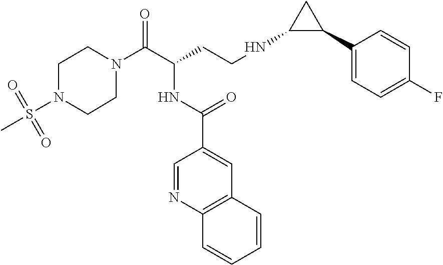 [SMILES: CS(=O)(=O)N1CCN(C(=O)[C@H](CCN[C@@H]2C[C@H]2c2ccc(F)cc2)NC(=O)c2cnc3ccccc3c2)CC1] |
| 12 | 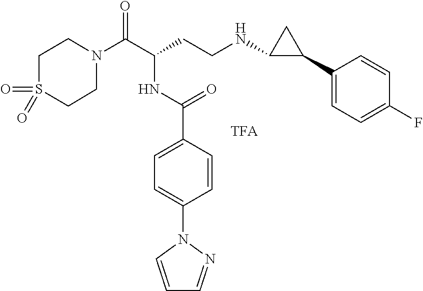 [SMILES: O=C(N[C@@H](CCN[C@@H]1C[C@H]1c1ccc(F)cc1)C(=O)N1CCS(=O)(=O)CC1)c1ccc(-n2cccn2)cc1] |
| 13 | 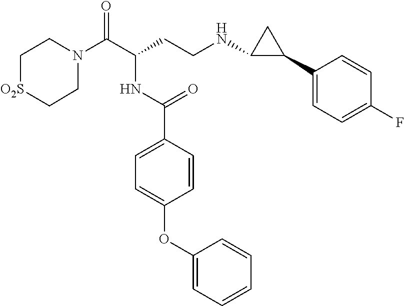 [SMILES: O=C(N[C@@H](CCN[C@@H]1C[C@H]1c1ccc(F)cc1)C(=O)N1CCS(=O)(=O)CC1)c1ccc(Oc2ccccc2)cc1] |
| 14 | 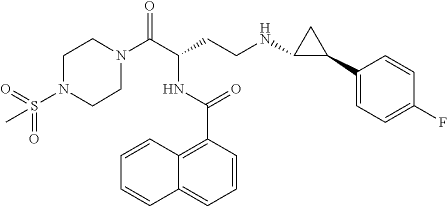 [SMILES: CS(=O)(=O)N1CCN(C(=O)[C@H](CCN[C@@H]2C[C@H]2c2ccc(F)cc2)NC(=O)c2cccc3ccccc23)CC1] |
| 15 | 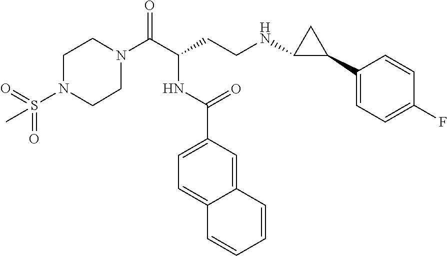 [SMILES: CS(=O)(=O)N1CCN(C(=O)[C@H](CCN[C@@H]2C[C@H]2c2ccc(F)cc2)NC(=O)c2ccc3ccccc3c2)CC1] |
| 16 | 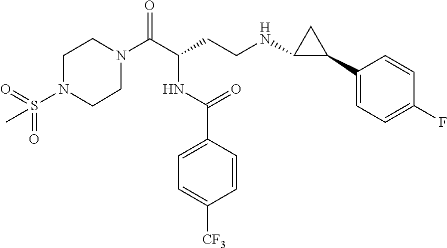 [SMILES: CS(=O)(=O)N1CCN(C(=O)[C@H](CCN[C@@H]2C[C@H]2c2ccc(F)cc2)NC(=O)c2ccc(C(F)(F)F)cc2)CC1] |
| 17 | 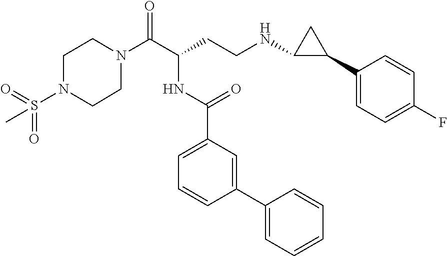 [SMILES: CS(=O)(=O)N1CCN(C(=O)[C@H](CCN[C@@H]2C[C@H]2c2ccc(F)cc2)NC(=O)c2cccc(-c3ccccc3)c2)CC1] |
| 18 | 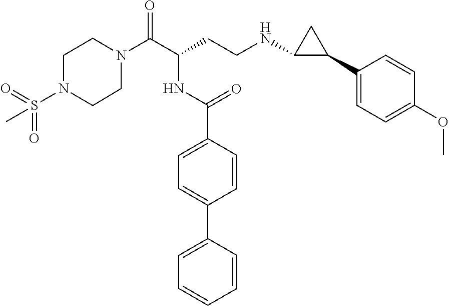 |
| 19 | 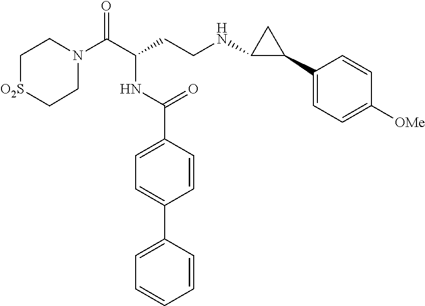 [SMILES: COc1ccc([C@@H]2C[C@H]2NCC[C@H](NC(=O)c2ccc(-c3ccccc3)cc2)C(=O)N2CCS(=O)(=O)CC2)cc1] |
| 20 | 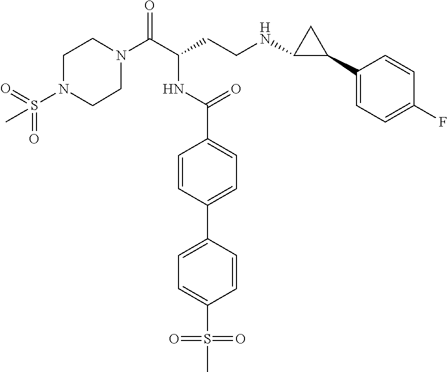 [SMILES: CS(=O)(=O)c1ccc(-c2ccc(C(=O)N[C@@H](CCN[C@@H]3C[C@H]3c3ccc(F)cc3)C(=O)N3CCN(S(C)(=O)=O)CC3)cc2)cc1] |
| 21 | 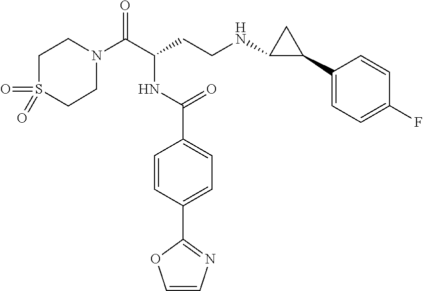 [SMILES: O=C(N[C@@H](CCN[C@@H]1C[C@H]1c1ccc(F)cc1)C(=O)N1CCS(=O)(=O)CC1)c1ccc(-c2ncco2)cc1] |
| 22 | 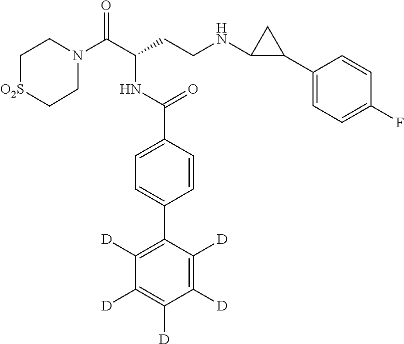 [SMILES: [2H]c1c([2H])c([2H])c(-c2ccc(C(=O)N[C@@H](CCNC3CC3c3ccc(F)cc3)C(=O)N3CCS(=O)(=O)CC3)cc2)c([2H])c1[2H]] |
| 23 | 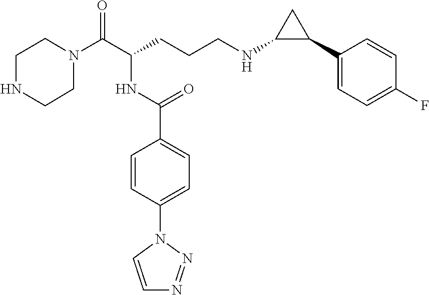 [SMILES: O=C(N[C@@H](CCCN[C@@H]1C[C@H]1c1ccc(F)cc1)C(=O)N1CCNCC1)c1ccc(-n2ccnn2)cc1] |
| 24 | 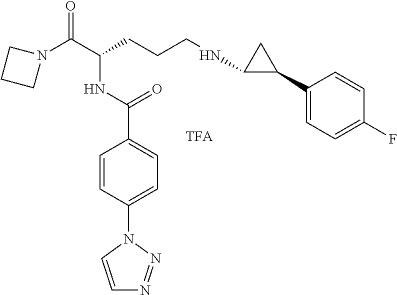 [SMILES: O=C(N[C@@H](CCCN[C@@H]1C[C@H]1c1ccc(F)cc1)C(=O)N1CCC1)c1ccc(-n2ccnn2)cc1] |
| 25 | 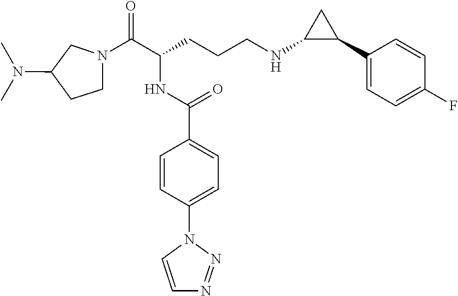 [SMILES: CN(C)C1CCN(C(=O)[C@H](CCCN[C@@H]2C[C@H]2c2ccc(F)cc2)NC(=O)c2ccc(-n3ccnn3)cc2)C1] |
| 26 | 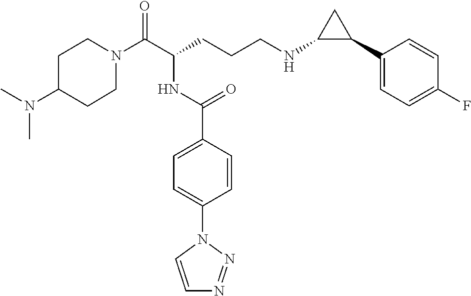 [SMILES: CN(C)C1CCN(C(=O)[C@H](CCCN[C@@H]2C[C@H]2c2ccc(F)cc2)NC(=O)c2ccc(-n3ccnn3)cc2)CC1] |
| 27 | 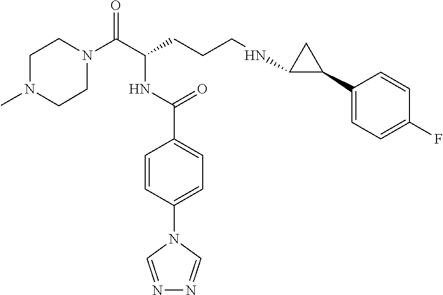 [SMILES: CN1CCN(C(=O)[C@H](CCCN[C@@H]2C[C@H]2c2ccc(F)cc2)NC(=O)c2ccc(-n3cnnc3)cc2)CC1] |
| 28 | 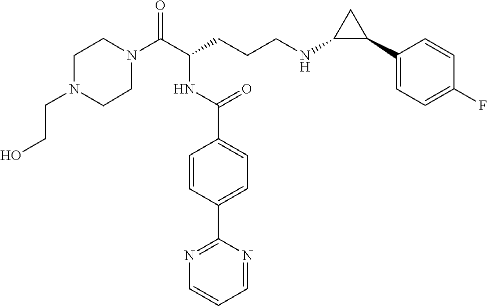 [SMILES: O=C(N[C@@H](CCCN[C@@H]1C[C@H]1c1ccc(F)cc1)C(=O)N1CCN(CCO)CC1)c1ccc(-c2ncccn2)cc1] |
| 29 | 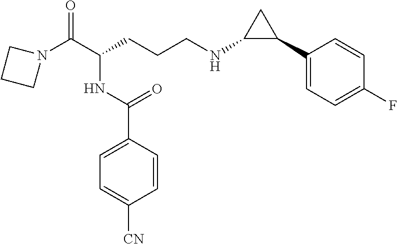 [SMILES: N#Cc1ccc(C(=O)N[C@@H](CCCN[C@@H]2C[C@H]2c2ccc(F)cc2)C(=O)N2CCC2)cc1] |
| 30 | 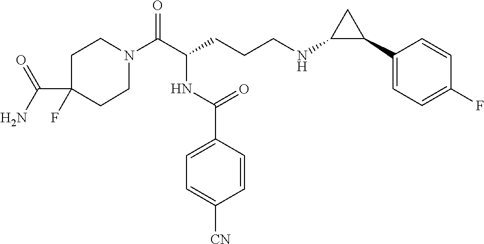 [SMILES: N#Cc1ccc(C(=O)N[C@@H](CCCN[C@@H]2C[C@H]2c2ccc(F)cc2)C(=O)N2CCC(F)(C(N)=O)CC2)cc1] |
| 31 | 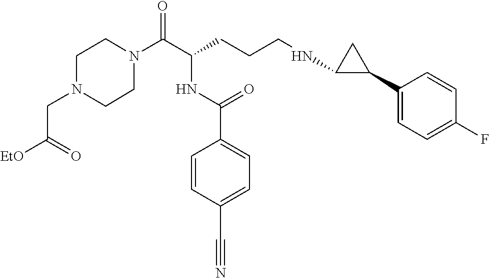 [SMILES: CCOC(=O)CN1CCN(C(=O)[C@H](CCCN[C@@H]2C[C@H]2c2ccc(F)cc2)NC(=O)c2ccc(C#N)cc2)CC1] |
| 32 | 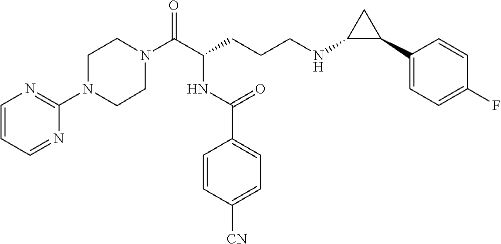 [SMILES: N#Cc1ccc(C(=O)N[C@@H](CCCN[C@@H]2C[C@H]2c2ccc(F)cc2)C(=O)N2CCN(c3ncccn3)CC2)cc1] |
| 33 | 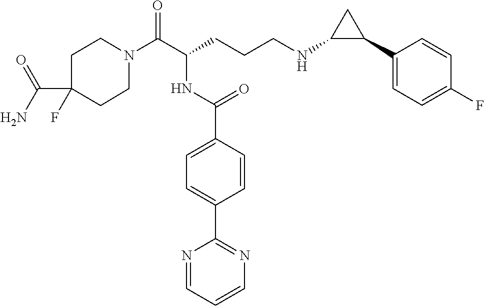 [SMILES: NC(=O)C1(F)CCN(C(=O)[C@H](CCCN[C@@H]2C[C@H]2c2ccc(F)cc2)NC(=O)c2ccc(-c3ncccn3)cc2)CC1] |
| 34 | 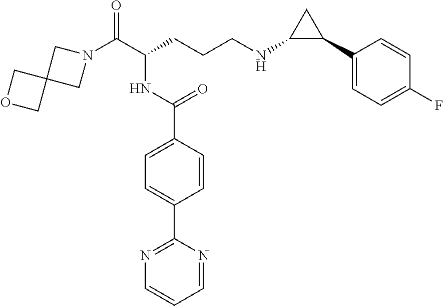 [SMILES: O=C(N[C@@H](CCCN[C@@H]1C[C@H]1c1ccc(F)cc1)C(=O)N1CC2(COC2)C1)c1ccc(-c2ncccn2)cc1] |
| 35 | 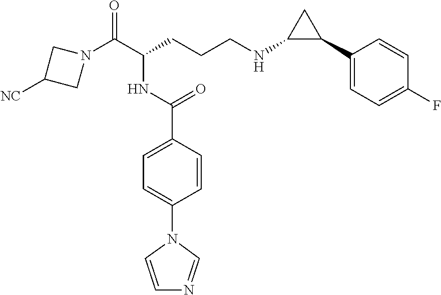 [SMILES: N#CC1CN(C(=O)[C@H](CCCN[C@@H]2C[C@H]2c2ccc(F)cc2)NC(=O)c2ccc(-n3ccnc3)cc2)C1] |
| 36 | 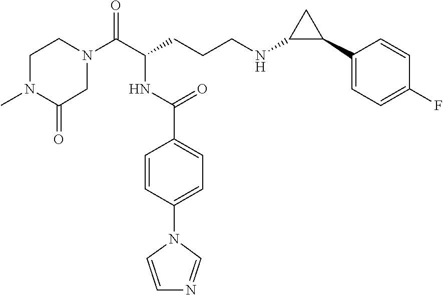 |
| 37 | 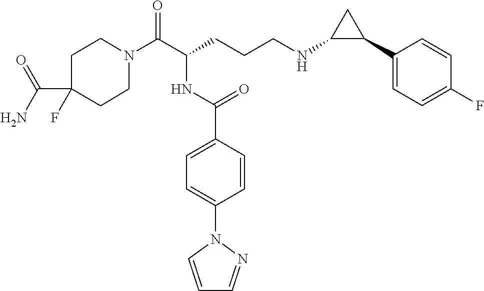 [SMILES: NC(=O)C1(F)CCN(C(=O)[C@H](CCCN[C@@H]2C[C@H]2c2ccc(F)cc2)NC(=O)c2ccc(-n3cccn3)cc2)CC1] |
| 38 | 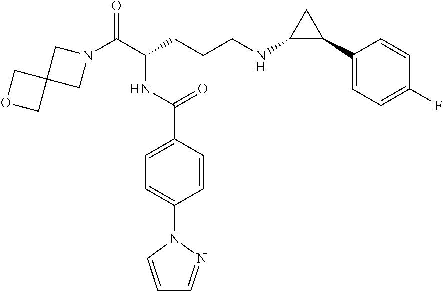 [SMILES: O=C(N[C@@H](CCCN[C@@H]1C[C@H]1c1ccc(F)cc1)C(=O)N1CC2(COC2)C1)c1ccc(-n2cccn2)cc1] |
| 41 | 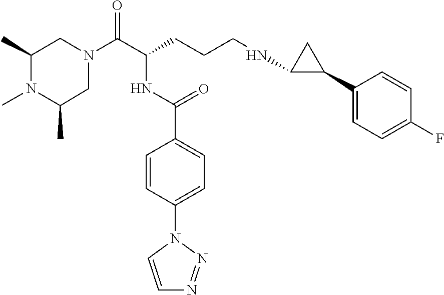 [SMILES: C[C@@H]1CN(C(=O)[C@H](CCCN[C@@H]2C[C@H]2c2ccc(F)cc2)NC(=O)c2ccc(-n3ccnn3)cc2)C[C@H](C)N1C] |
| 42 | 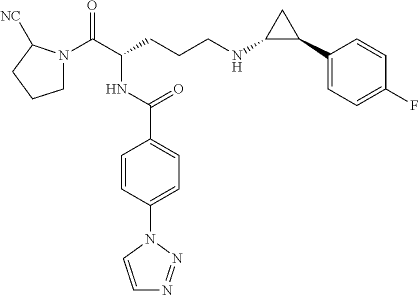 [SMILES: N#CC1CCCN1C(=O)[C@H](CCCN[C@@H]1C[C@H]1c1ccc(F)cc1)NC(=O)c1ccc(-n2ccnn2)cc1] |
| 43 | 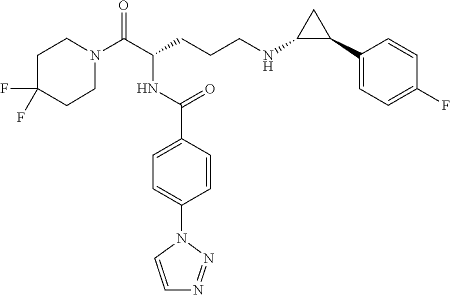 [SMILES: O=C(N[C@@H](CCCN[C@@H]1C[C@H]1c1ccc(F)cc1)C(=O)N1CCC(F)(F)CC1)c1ccc(-n2ccnn2)cc1] |
| 44 | 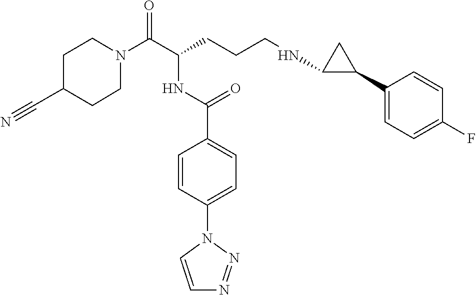 [SMILES: N#CC1CCN(C(=O)[C@H](CCCN[C@@H]2C[C@H]2c2ccc(F)cc2)NC(=O)c2ccc(-n3ccnn3)cc2)CC1] |
| 45 | 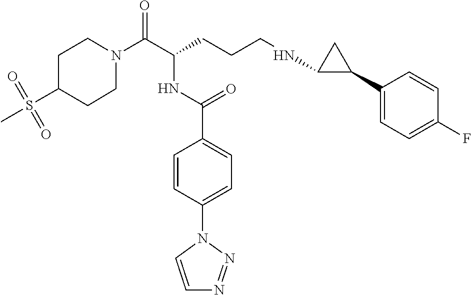 [SMILES: CS(=O)(=O)C1CCN(C(=O)[C@H](CCCN[C@@H]2C[C@H]2c2ccc(F)cc2)NC(=O)c2ccc(-n3ccnn3)cc2)CC1] |
| 46 | 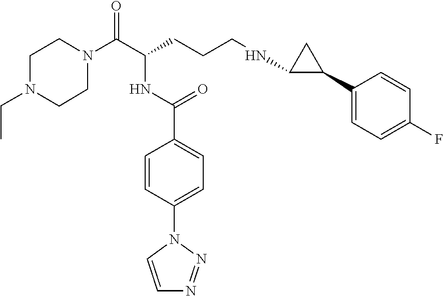 [SMILES: CCN1CCN(C(=O)[C@H](CCCN[C@@H]2C[C@H]2c2ccc(F)cc2)NC(=O)c2ccc(-n3ccnn3)cc2)CC1] |
| 47 | 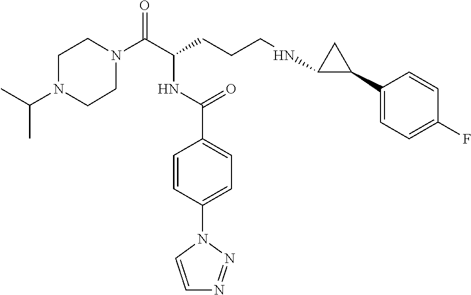 [SMILES: CC(C)N1CCN(C(=O)[C@H](CCCN[C@@H]2C[C@H]2c2ccc(F)cc2)NC(=O)c2ccc(-n3ccnn3)cc2)CC1] |
| 48 | 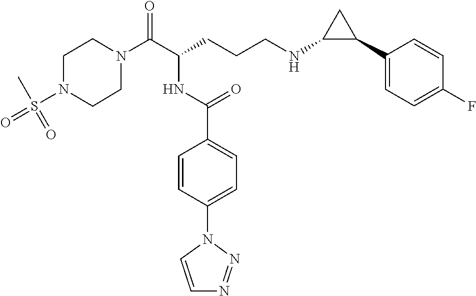 [SMILES: CS(=O)(=O)N1CCN(C(=O)[C@H](CCCN[C@@H]2C[C@H]2c2ccc(F)cc2)NC(=O)c2ccc(-n3ccnn3)cc2)CC1] |
| 49 | 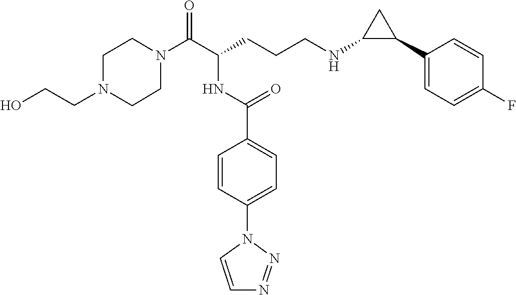 [SMILES: O=C(N[C@@H](CCCN[C@@H]1C[C@H]1c1ccc(F)cc1)C(=O)N1CCN(CCO)CC1)c1ccc(-n2ccnn2)cc1] |
| 50 | 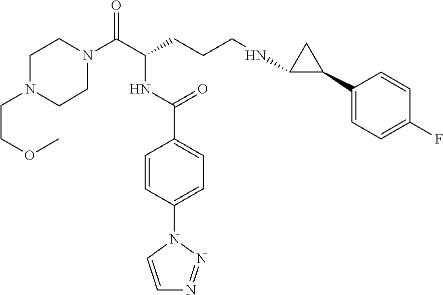 [SMILES: COCCN1CCN(C(=O)[C@H](CCCN[C@@H]2C[C@H]2c2ccc(F)cc2)NC(=O)c2ccc(-n3ccnn3)cc2)CC1] |
| 51 | 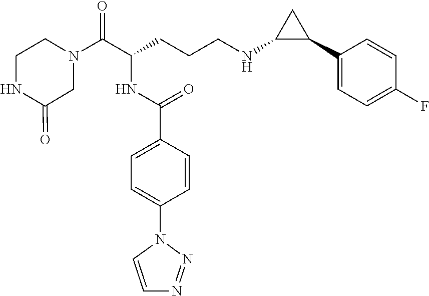 [SMILES: O=C1CN(C(=O)[C@H](CCCN[C@@H]2C[C@H]2c2ccc(F)cc2)NC(=O)c2ccc(-n3ccnn3)cc2)CCN1] |
| 52 | 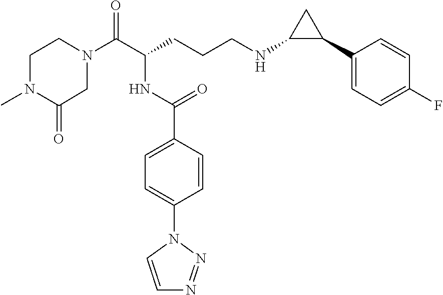 [SMILES: CN1CCN(C(=O)[C@H](CCCN[C@@H]2C[C@H]2c2ccc(F)cc2)NC(=O)c2ccc(-n3ccnn3)cc2)CC1=O] |
| 53 | 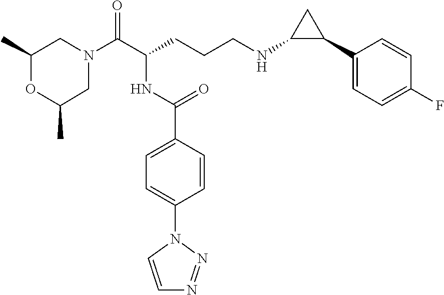 [SMILES: C[C@@H]1CN(C(=O)[C@H](CCCN[C@@H]2C[C@H]2c2ccc(F)cc2)NC(=O)c2ccc(-n3ccnn3)cc2)C[C@H](C)O1] |
| 54 | 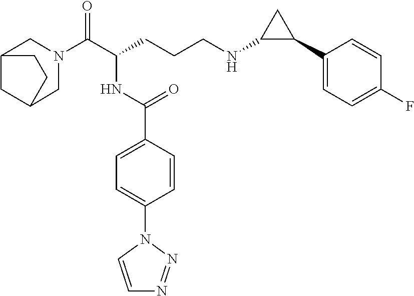 |
| 55 | 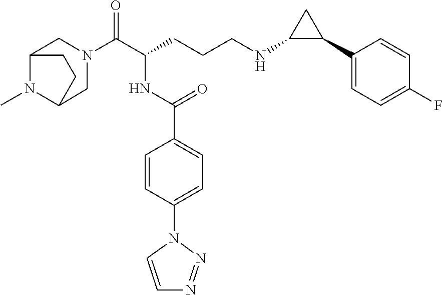 [SMILES: CN1C2CCC1CN(C(=O)[C@H](CCCN[C@@H]1C[C@H]1c1ccc(F)cc1)NC(=O)c1ccc(-n3ccnn3)cc1)C2] |
| 56 | 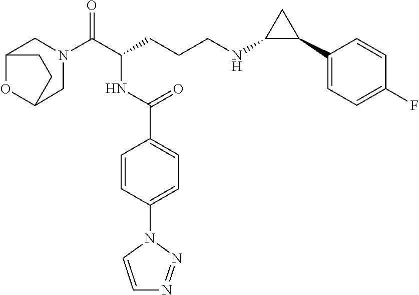 [SMILES: O=C(N[C@@H](CCCN[C@@H]1C[C@H]1c1ccc(F)cc1)C(=O)N1CC2CCC(C1)O2)c1ccc(-n2ccnn2)cc1] |
| 57 | 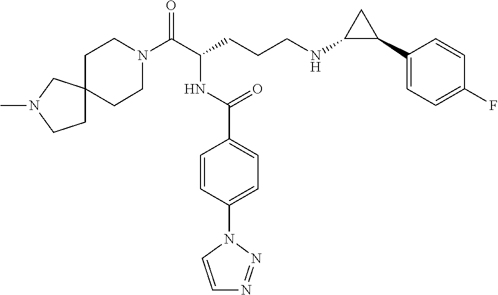 [SMILES: CN1CCC2(CCN(C(=O)[C@H](CCCN[C@@H]3C[C@H]3c3ccc(F)cc3)NC(=O)c3ccc(-n4ccnn4)cc3)CC2)C1] |
| 58 | 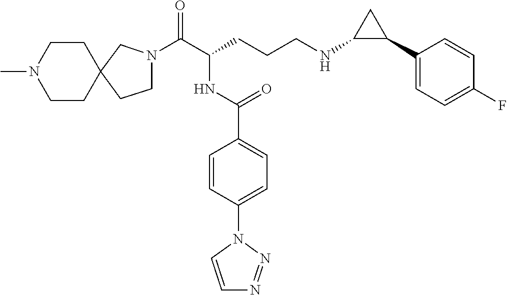 [SMILES: CN1CCC2(CC1)CCN(C(=O)[C@H](CCCN[C@@H]1C[C@H]1c1ccc(F)cc1)NC(=O)c1ccc(-n3ccnn3)cc1)C2] |
| 59 | 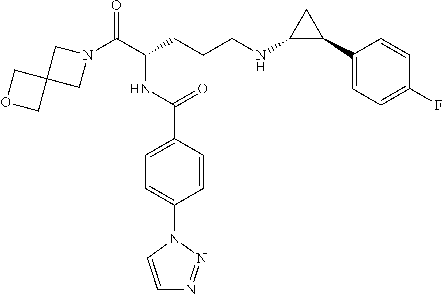 [SMILES: O=C(N[C@@H](CCCN[C@@H]1C[C@H]1c1ccc(F)cc1)C(=O)N1CC2(COC2)C1)c1ccc(-n2ccnn2)cc1] |
| 60 | 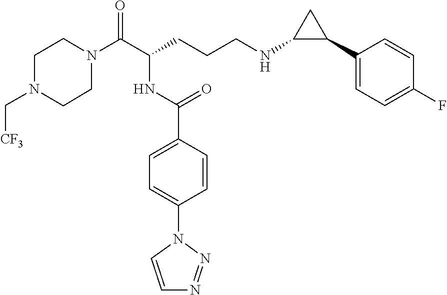 [SMILES: O=C(N[C@@H](CCCN[C@@H]1C[C@H]1c1ccc(F)cc1)C(=O)N1CCN(CC(F)(F)F)CC1)c1ccc(-n2ccnn2)cc1] |
| 61 | 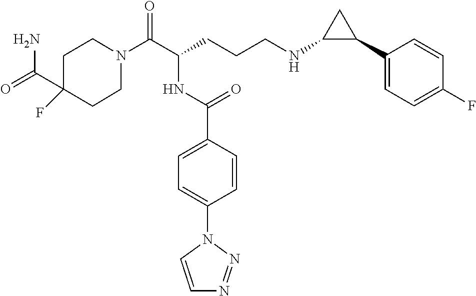 [SMILES: NC(=O)C1(F)CCN(C(=O)[C@H](CCCN[C@@H]2C[C@H]2c2ccc(F)cc2)NC(=O)c2ccc(-n3ccnn3)cc2)CC1] |
| 62 | 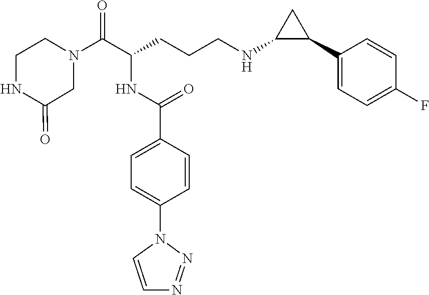 [SMILES: O=C1CN(C(=O)[C@H](CCCN[C@@H]2C[C@H]2c2ccc(F)cc2)NC(=O)c2ccc(-n3ccnn3)cc2)CCN1] |
| 63 | 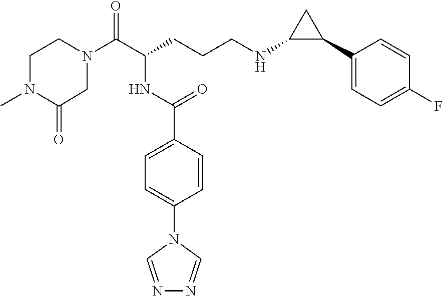 [SMILES: CN1CCN(C(=O)[C@H](CCCN[C@@H]2C[C@H]2c2ccc(F)cc2)NC(=O)c2ccc(-n3cnnc3)cc2)CC1=O] |
| 64 | 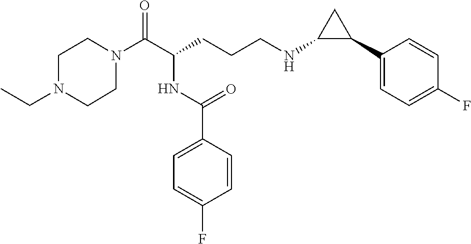 [SMILES: CCN1CCN(C(=O)[C@H](CCCN[C@@H]2C[C@H]2c2ccc(F)cc2)NC(=O)c2ccc(F)cc2)CC1] |
| 65 | 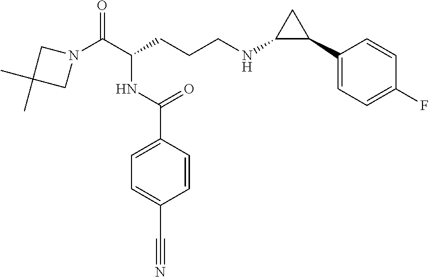 [SMILES: CC1(C)CN(C(=O)[C@H](CCCN[C@@H]2C[C@H]2c2ccc(F)cc2)NC(=O)c2ccc(C#N)cc2)C1] |
| 66 | 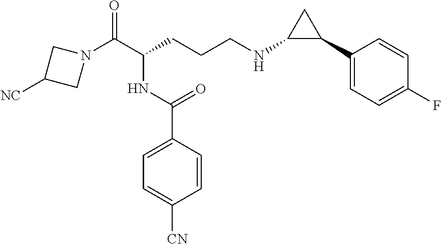 [SMILES: N#Cc1ccc(C(=O)N[C@@H](CCCN[C@@H]2C[C@H]2c2ccc(F)cc2)C(=O)N2CC(C#N)C2)cc1] |
| 67 | 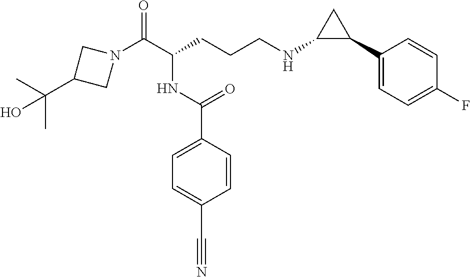 [SMILES: CC(C)(O)C1CN(C(=O)[C@H](CCCN[C@@H]2C[C@H]2c2ccc(F)cc2)NC(=O)c2ccc(C#N)cc2)C1] |
| 68 | 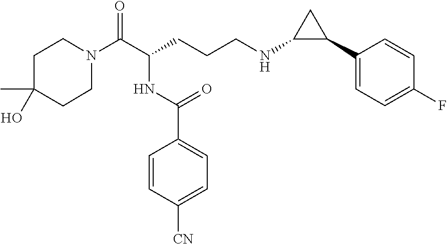 [SMILES: CC1(O)CCN(C(=O)[C@H](CCCN[C@@H]2C[C@H]2c2ccc(F)cc2)NC(=O)c2ccc(C#N)cc2)CC1] |
| 69 | 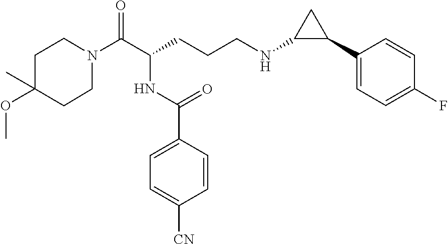 |
| 70 | 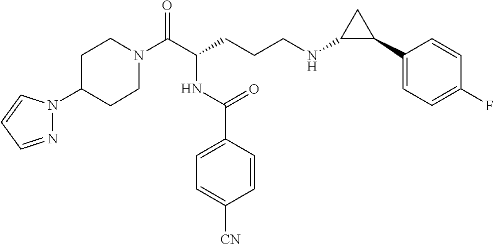 |
| 71 | 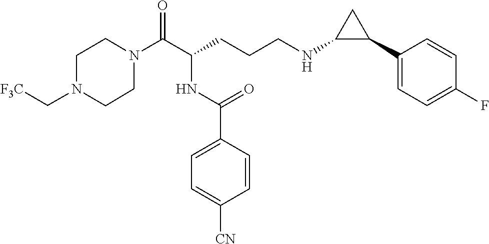 [SMILES: N#Cc1ccc(C(=O)N[C@@H](CCCN[C@@H]2C[C@H]2c2ccc(F)cc2)C(=O)N2CCN(CC(F)(F)F)CC2)cc1] |
| 72 | 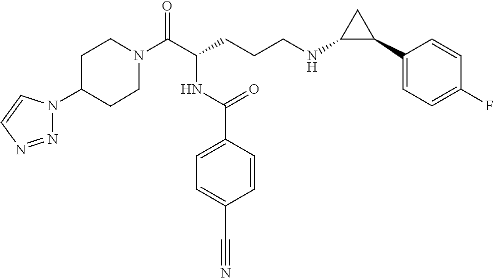 [SMILES: N#Cc1ccc(C(=O)N[C@@H](CCCN[C@@H]2C[C@H]2c2ccc(F)cc2)C(=O)N2CCC(n3ccnn3)CC2)cc1] |
| 73 | 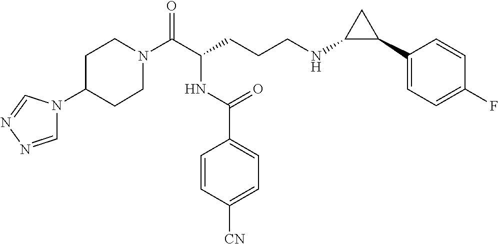 [SMILES: N#Cc1ccc(C(=O)N[C@@H](CCCN[C@@H]2C[C@H]2c2ccc(F)cc2)C(=O)N2CCC(n3cnnc3)CC2)cc1] |
| 74 | 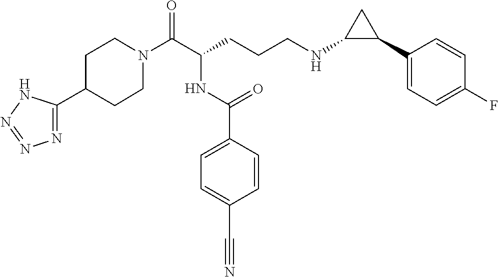 [SMILES: N#Cc1ccc(C(=O)N[C@@H](CCCN[C@@H]2C[C@H]2c2ccc(F)cc2)C(=O)N2CCC(c3nnn[nH]3)CC2)cc1] |
| 75 | 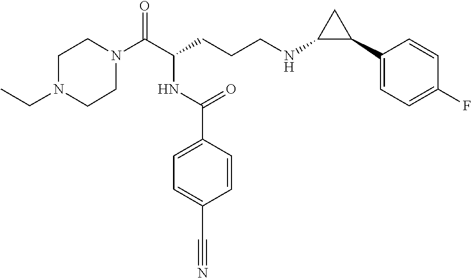 [SMILES: CCN1CCN(C(=O)[C@H](CCCN[C@@H]2C[C@H]2c2ccc(F)cc2)NC(=O)c2ccc(C#N)cc2)CC1] |
| 76 | 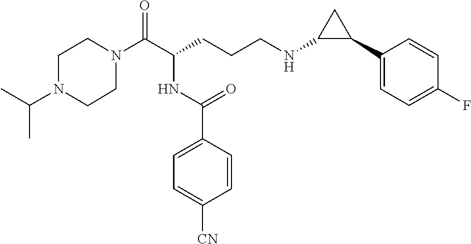 |
| 77 | 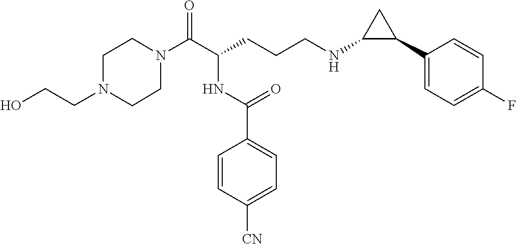 [SMILES: N#Cc1ccc(C(=O)N[C@@H](CCCN[C@@H]2C[C@H]2c2ccc(F)cc2)C(=O)N2CCN(CCO)CC2)cc1] |
| 78 | 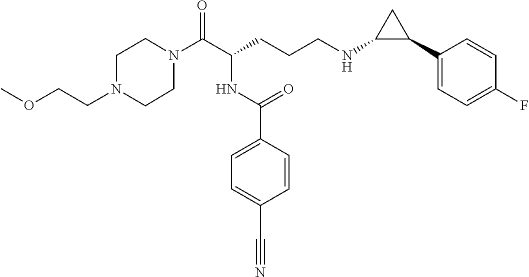 [SMILES: COCCN1CCN(C(=O)[C@H](CCCN[C@@H]2C[C@H]2c2ccc(F)cc2)NC(=O)c2ccc(C#N)cc2)CC1] |
| 79 | 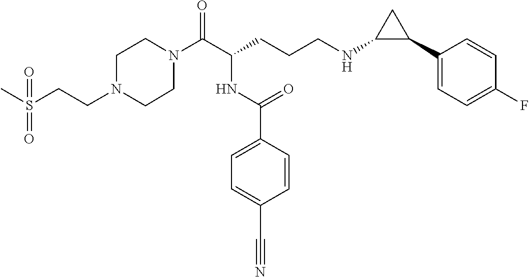 [SMILES: CS(=O)(=O)CCN1CCN(C(=O)[C@H](CCCN[C@@H]2C[C@H]2c2ccc(F)cc2)NC(=O)c2ccc(C#N)cc2)CC1] |
| 80 | 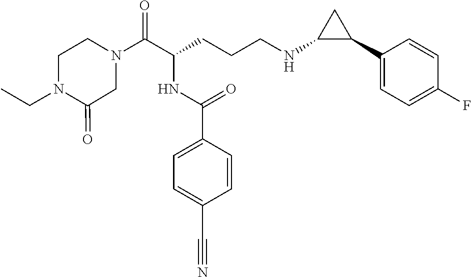 [SMILES: CCN1CCN(C(=O)[C@H](CCCN[C@@H]2C[C@H]2c2ccc(F)cc2)NC(=O)c2ccc(C#N)cc2)CC1=O] |
| 81 | 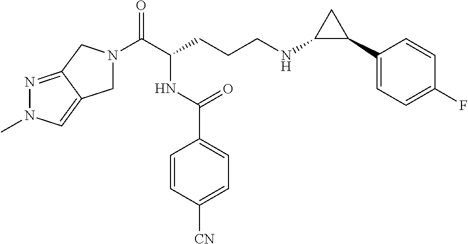 [SMILES: Cn1cc2c(n1)CN(C(=O)[C@H](CCCN[C@@H]1C[C@H]1c1ccc(F)cc1)NC(=O)c1ccc(C#N)cc1)C2] |
| 82 | 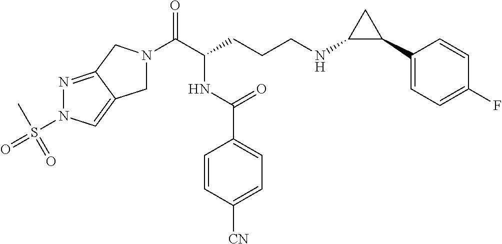 [SMILES: CS(=O)(=O)n1cc2c(n1)CN(C(=O)[C@H](CCCN[C@@H]1C[C@H]1c1ccc(F)cc1)NC(=O)c1ccc(C#N)cc1)C2] |
| 83 | 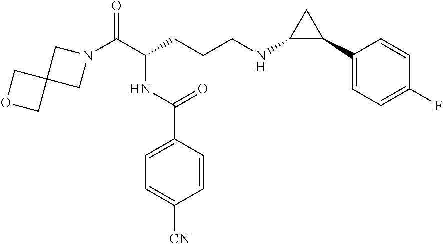 [SMILES: N#Cc1ccc(C(=O)N[C@@H](CCCN[C@@H]2C[C@H]2c2ccc(F)cc2)C(=O)N2CC3(COC3)C2)cc1] |
| 84 | 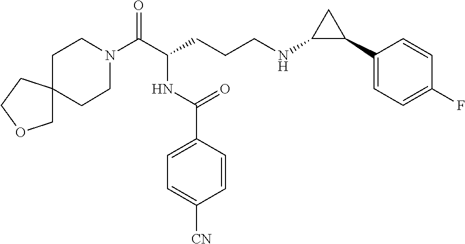 [SMILES: N#Cc1ccc(C(=O)N[C@@H](CCCN[C@@H]2C[C@H]2c2ccc(F)cc2)C(=O)N2CCC3(CCOC3)CC2)cc1] |
| 85 | 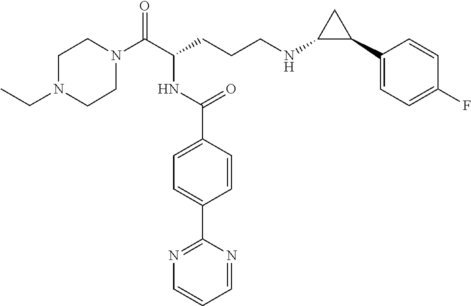 [SMILES: CCN1CCN(C(=O)[C@H](CCCN[C@@H]2C[C@H]2c2ccc(F)cc2)NC(=O)c2ccc(-c3ncccn3)cc2)CC1] |
| 86 | 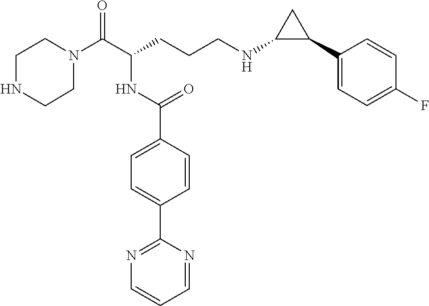 [SMILES: O=C(N[C@@H](CCCN[C@@H]1C[C@H]1c1ccc(F)cc1)C(=O)N1CCNCC1)c1ccc(-c2ncccn2)cc1] |
| 89 | 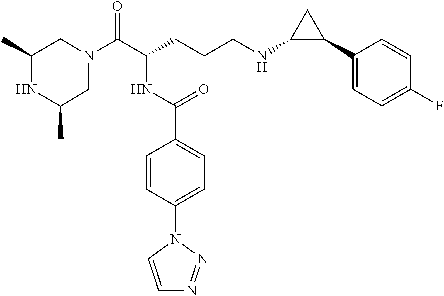 [SMILES: C[C@@H]1CN(C(=O)[C@H](CCCN[C@@H]2C[C@H]2c2ccc(F)cc2)NC(=O)c2ccc(-n3ccnn3)cc2)C[C@H](C)N1] |
| 90 | 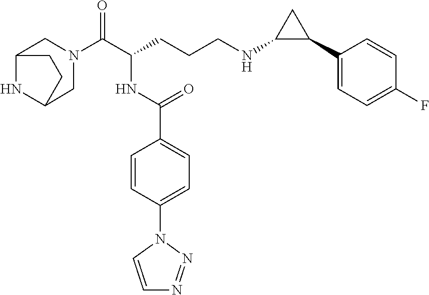 [SMILES: O=C(N[C@@H](CCCN[C@@H]1C[C@H]1c1ccc(F)cc1)C(=O)N1CC2CCC(C1)N2)c1ccc(-n2ccnn2)cc1] |
| 91 | 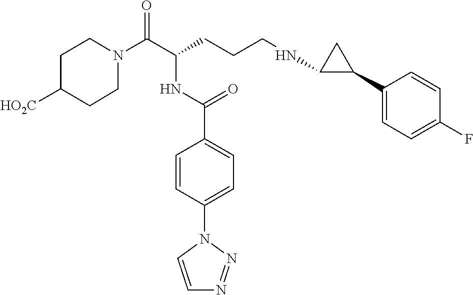 [SMILES: O=C(N[C@@H](CCCN[C@@H]1C[C@H]1c1ccc(F)cc1)C(=O)N1CCC(C(=O)O)CC1)c1ccc(-n2ccnn2)cc1] |
| 92 | 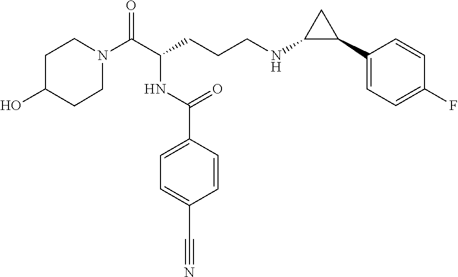 [SMILES: N#Cc1ccc(C(=O)N[C@@H](CCCN[C@@H]2C[C@H]2c2ccc(F)cc2)C(=O)N2CCC(O)CC2)cc1] |
| 93 | 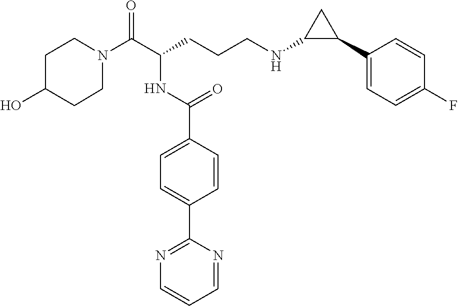 |
| 94 | 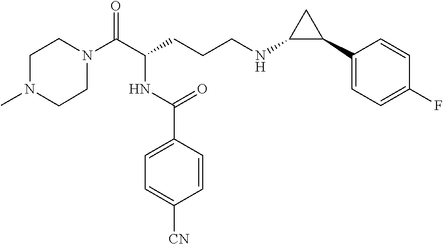 [SMILES: CN1CCN(C(=O)[C@H](CCCN[C@@H]2C[C@H]2c2ccc(F)cc2)NC(=O)c2ccc(C#N)cc2)CC1] |
| 95 | 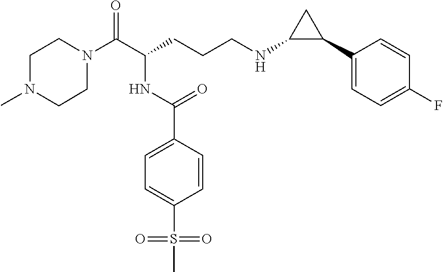 [SMILES: CN1CCN(C(=O)[C@H](CCCN[C@@H]2C[C@H]2c2ccc(F)cc2)NC(=O)c2ccc(S(C)(=O)=O)cc2)CC1] |
| 96 | 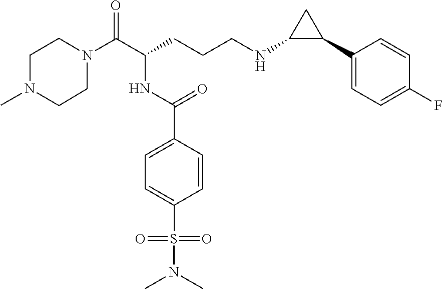 [SMILES: CN1CCN(C(=O)[C@H](CCCN[C@@H]2C[C@H]2c2ccc(F)cc2)NC(=O)c2ccc(S(=O)(=O)N(C)C)cc2)CC1] |
| 97 | 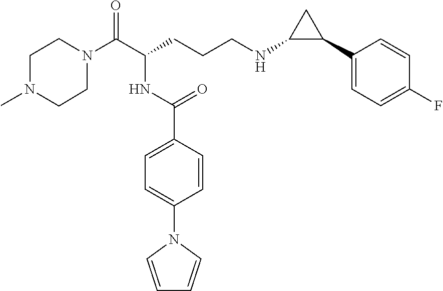 [SMILES: CN1CCN(C(=O)[C@H](CCCN[C@@H]2C[C@H]2c2ccc(F)cc2)NC(=O)c2ccc(-n3cccc3)cc2)CC1] |
| 98 | 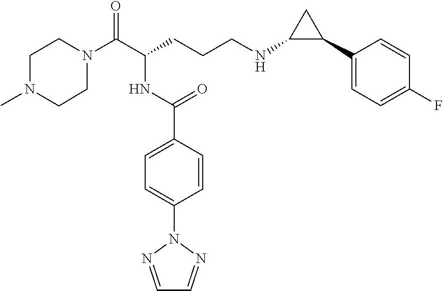 [SMILES: CN1CCN(C(=O)[C@H](CCCN[C@@H]2C[C@H]2c2ccc(F)cc2)NC(=O)c2ccc(-n3nccn3)cc2)CC1] |
| 99 | 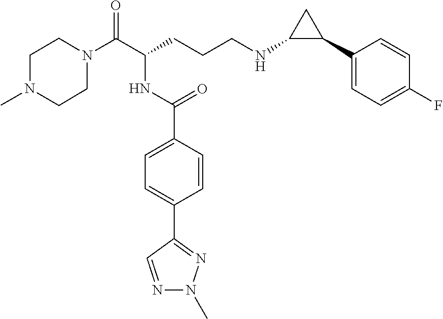 [SMILES: CN1CCN(C(=O)[C@H](CCCN[C@@H]2C[C@H]2c2ccc(F)cc2)NC(=O)c2ccc(-c3cnn(C)n3)cc2)CC1] |
| 100 | 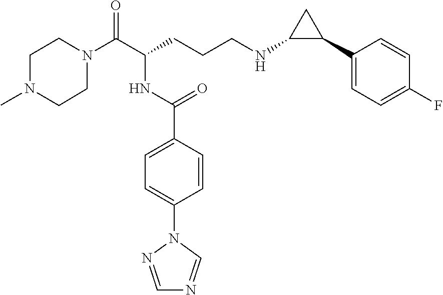 [SMILES: CN1CCN(C(=O)[C@H](CCCN[C@@H]2C[C@H]2c2ccc(F)cc2)NC(=O)c2ccc(-n3cncn3)cc2)CC1] |
| 101 | 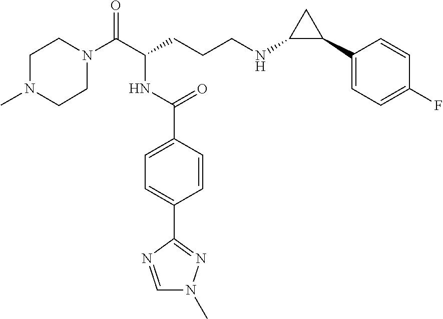 [SMILES: CN1CCN(C(=O)[C@H](CCCN[C@@H]2C[C@H]2c2ccc(F)cc2)NC(=O)c2ccc(-c3ncn(C)n3)cc2)CC1] |
| 102 | 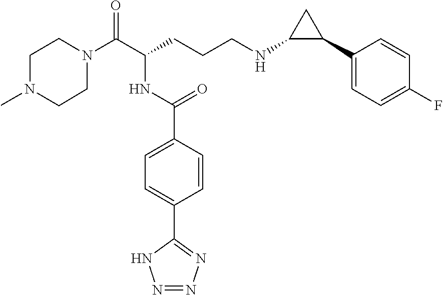 [SMILES: CN1CCN(C(=O)[C@H](CCCN[C@@H]2C[C@H]2c2ccc(F)cc2)NC(=O)c2ccc(-c3nnn[nH]3)cc2)CC1] |
| 103 | 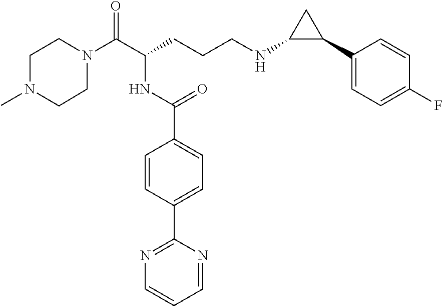 [SMILES: CN1CCN(C(=O)[C@H](CCCN[C@@H]2C[C@H]2c2ccc(F)cc2)NC(=O)c2ccc(-c3ncccn3)cc2)CC1] |
| 104 | 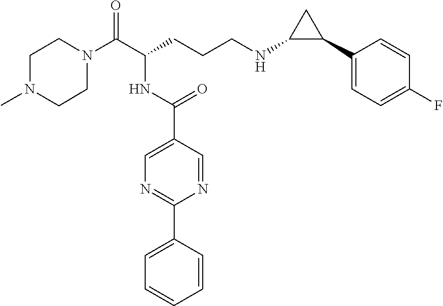 [SMILES: CN1CCN(C(=O)[C@H](CCCN[C@@H]2C[C@H]2c2ccc(F)cc2)NC(=O)c2cnc(-c3ccccc3)nc2)CC1] |
| 105 | 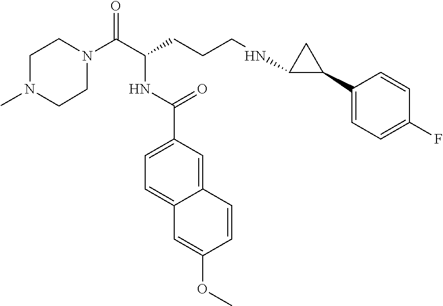 [SMILES: COc1ccc2cc(C(=O)N[C@@H](CCCN[C@@H]3C[C@H]3c3ccc(F)cc3)C(=O)N3CCN(C)CC3)ccc2c1] |
| 106 | 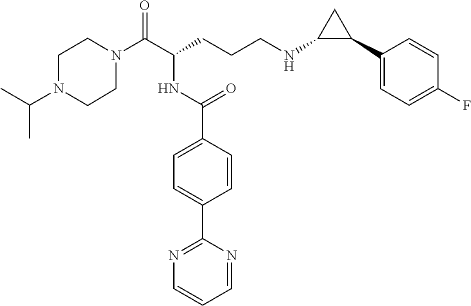 [SMILES: CC(C)N1CCN(C(=O)[C@H](CCCN[C@@H]2C[C@H]2c2ccc(F)cc2)NC(=O)c2ccc(-c3ncccn3)cc2)CC1] |
| 107 | 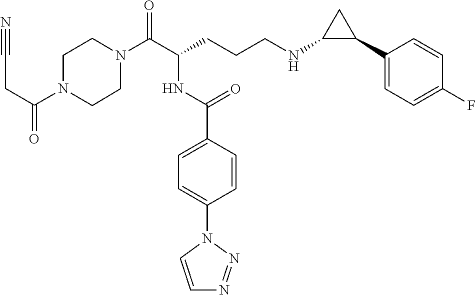 [SMILES: N#CCC(=O)N1CCN(C(=O)[C@H](CCCN[C@@H]2C[C@H]2c2ccc(F)cc2)NC(=O)c2ccc(-n3ccnn3)cc2)CC1] |
| 108 | 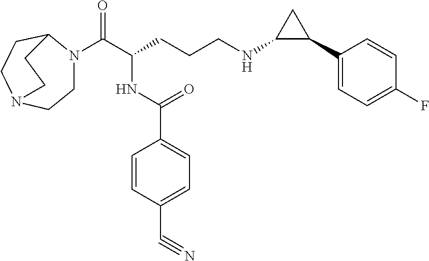 [SMILES: N#Cc1ccc(C(=O)N[C@@H](CCCN[C@@H]2C[C@H]2c2ccc(F)cc2)C(=O)N2CCN3CCC2CC3)cc1] |
| 109 | 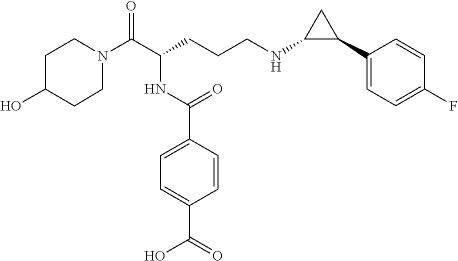 [SMILES: O=C(O)c1ccc(C(=O)N[C@@H](CCCN[C@@H]2C[C@H]2c2ccc(F)cc2)C(=O)N2CCC(O)CC2)cc1] |
| 110 | 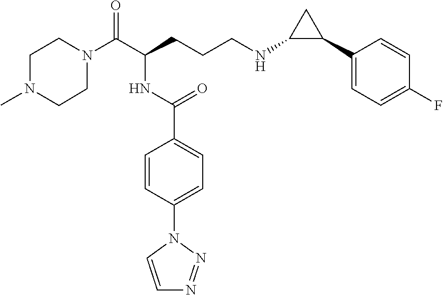 [SMILES: CN1CCN(C(=O)[C@@H](CCCN[C@@H]2C[C@H]2c2ccc(F)cc2)NC(=O)c2ccc(-n3ccnn3)cc2)CC1] |
| 111 | 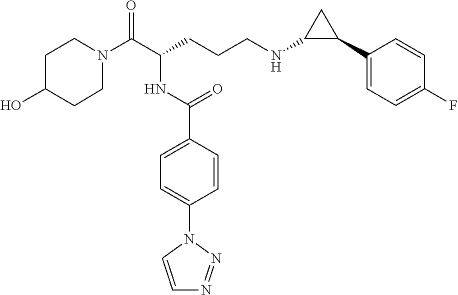 |
| 112 | 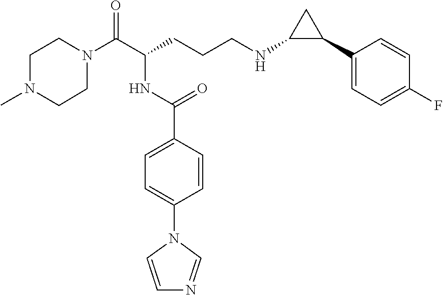 [SMILES: CN1CCN(C(=O)[C@H](CCCN[C@@H]2C[C@H]2c2ccc(F)cc2)NC(=O)c2ccc(-n3ccnc3)cc2)CC1] |
| 113 | 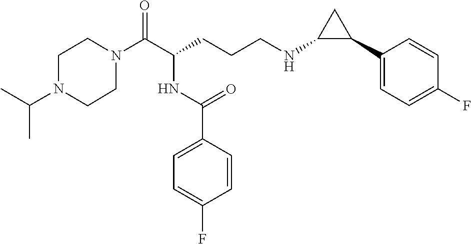 [SMILES: CC(C)N1CCN(C(=O)[C@H](CCCN[C@@H]2C[C@H]2c2ccc(F)cc2)NC(=O)c2ccc(F)cc2)CC1] |
| 114 | 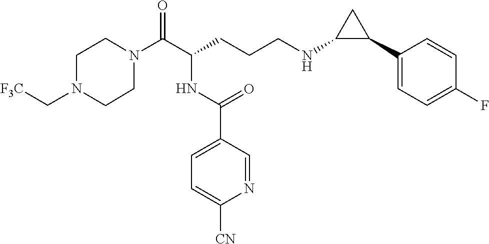 [SMILES: N#Cc1ccc(C(=O)N[C@@H](CCCN[C@@H]2C[C@H]2c2ccc(F)cc2)C(=O)N2CCN(CC(F)(F)F)CC2)cn1] |
| 115 | 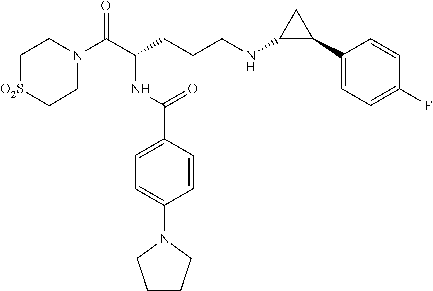 |
| 116 | 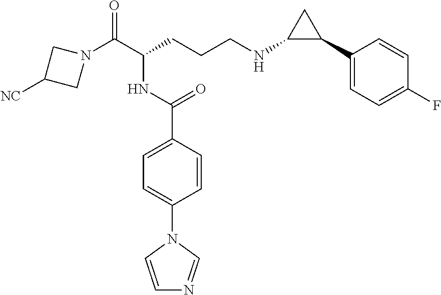 [SMILES: N#CC1CN(C(=O)[C@H](CCCN[C@@H]2C[C@H]2c2ccc(F)cc2)NC(=O)c2ccc(-n3ccnc3)cc2)C1] |
| 117 | 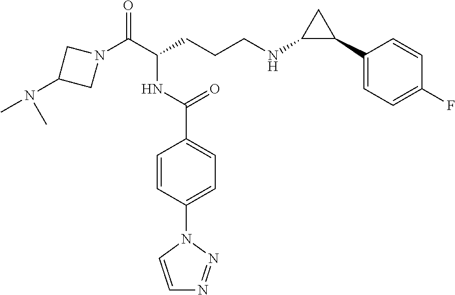 [SMILES: CN(C)C1CN(C(=O)[C@H](CCCN[C@@H]2C[C@H]2c2ccc(F)cc2)NC(=O)c2ccc(-n3ccnn3)cc2)C1] |
| 118 | 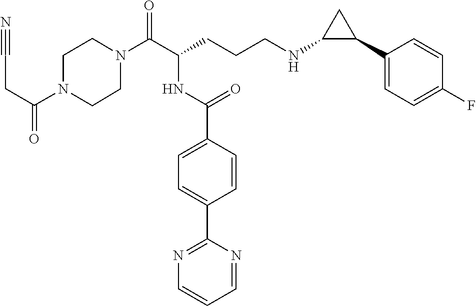 [SMILES: N#CCC(=O)N1CCN(C(=O)[C@H](CCCN[C@@H]2C[C@H]2c2ccc(F)cc2)NC(=O)c2ccc(-c3ncccn3)cc2)CC1] |
| 119 | 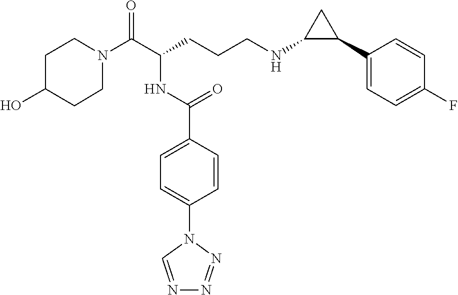 [SMILES: O=C(N[C@@H](CCCN[C@@H]1C[C@H]1c1ccc(F)cc1)C(=O)N1CCC(O)CC1)c1ccc(-n2cnnn2)cc1] |
| 120 | 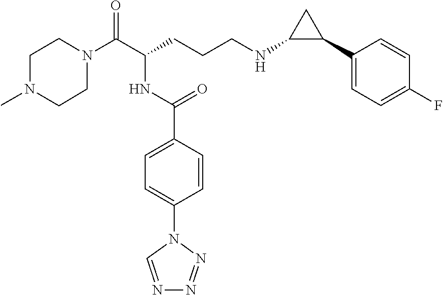 [SMILES: CN1CCN(C(=O)[C@H](CCCN[C@@H]2C[C@H]2c2ccc(F)cc2)NC(=O)c2ccc(-n3cnnn3)cc2)CC1] |
| 121 | 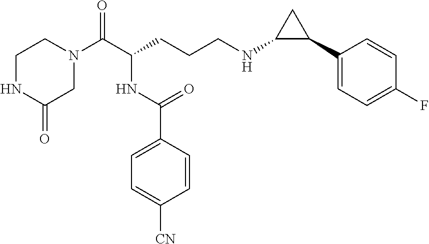 [SMILES: N#Cc1ccc(C(=O)N[C@@H](CCCN[C@@H]2C[C@H]2c2ccc(F)cc2)C(=O)N2CCNC(=O)C2)cc1] |
| 122 | 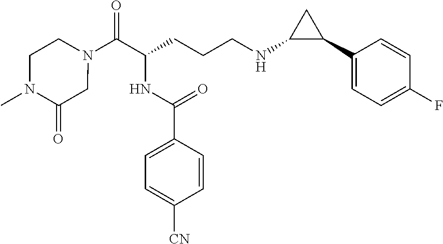 [SMILES: CN1CCN(C(=O)[C@H](CCCN[C@@H]2C[C@H]2c2ccc(F)cc2)NC(=O)c2ccc(C#N)cc2)CC1=O] |
| 123 | 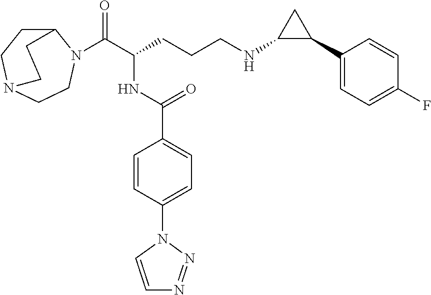 [SMILES: O=C(N[C@@H](CCCN[C@@H]1C[C@H]1c1ccc(F)cc1)C(=O)N1CCN2CCC1CC2)c1ccc(-n2ccnn2)cc1] |
| 124 | 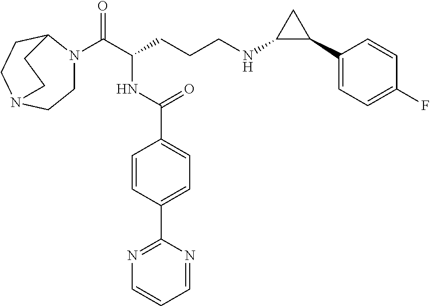 [SMILES: O=C(N[C@@H](CCCN[C@@H]1C[C@H]1c1ccc(F)cc1)C(=O)N1CCN2CCC1CC2)c1ccc(-c2ncccn2)cc1] |
| 125 | 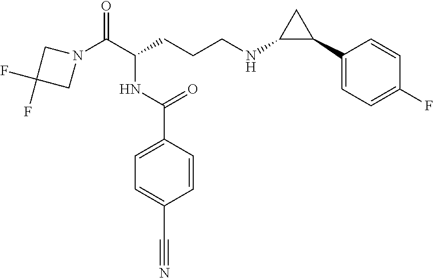 [SMILES: N#Cc1ccc(C(=O)N[C@@H](CCCN[C@@H]2C[C@H]2c2ccc(F)cc2)C(=O)N2CC(F)(F)C2)cc1] |
| 126 | 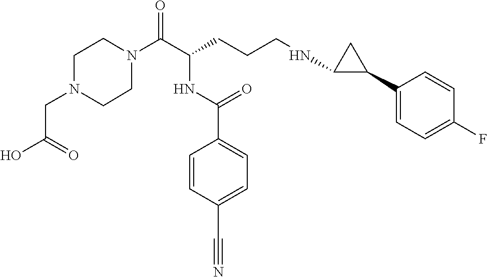 [SMILES: N#Cc1ccc(C(=O)N[C@@H](CCCN[C@@H]2C[C@H]2c2ccc(F)cc2)C(=O)N2CCN(CC(=O)O)CC2)cc1] |
| 127 | 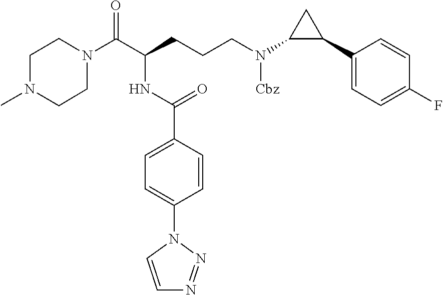 [SMILES: CN1CCN(C(=O)[C@@H](CCCN(C(=O)OCc2ccccc2)[C@@H]2C[C@H]2c2ccc(F)cc2)NC(=O)c2ccc(-n3ccnn3)cc2)CC1] |
| 128 | 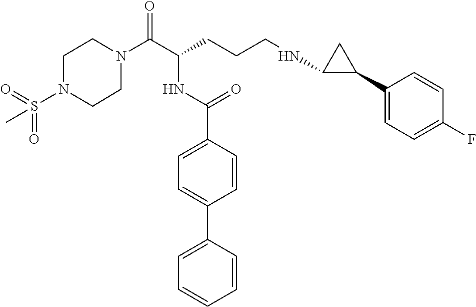 [SMILES: CS(=O)(=O)N1CCN(C(=O)[C@H](CCCN[C@@H]2C[C@H]2c2ccc(F)cc2)NC(=O)c2ccc(-c3ccccc3)cc2)CC1] |
| 129 | 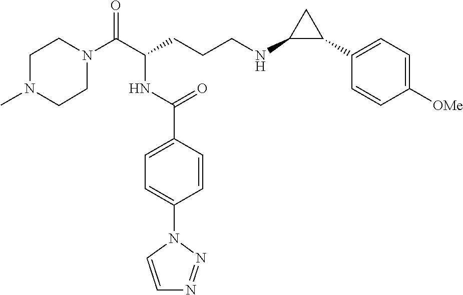 [SMILES: COc1ccc([C@H]2C[C@@H]2NCCC[C@H](NC(=O)c2ccc(-n3ccnn3)cc2)C(=O)N2CCN(C)CC2)cc1] |
| 130 | 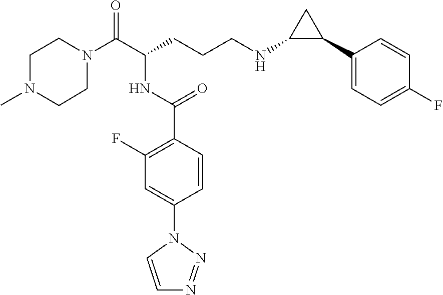 [SMILES: CN1CCN(C(=O)[C@H](CCCN[C@@H]2C[C@H]2c2ccc(F)cc2)NC(=O)c2ccc(-n3ccnn3)cc2F)CC1] |
| 131 | 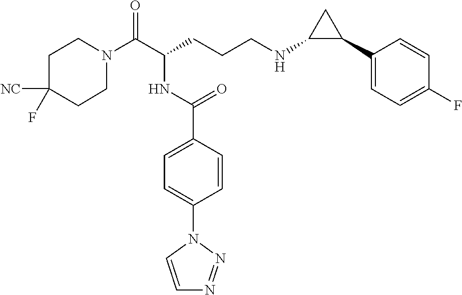 [SMILES: N#CC1(F)CCN(C(=O)[C@H](CCCN[C@@H]2C[C@H]2c2ccc(F)cc2)NC(=O)c2ccc(-n3ccnn3)cc2)CC1] |
| 132 | 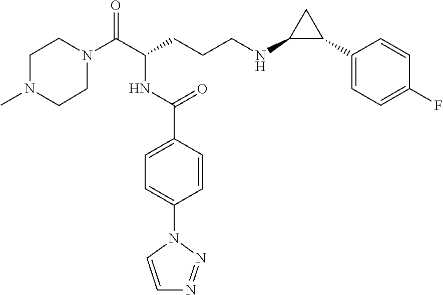 [SMILES: CN1CCN(C(=O)[C@H](CCCN[C@H]2C[C@@H]2c2ccc(F)cc2)NC(=O)c2ccc(-n3ccnn3)cc2)CC1] |
| 133 | 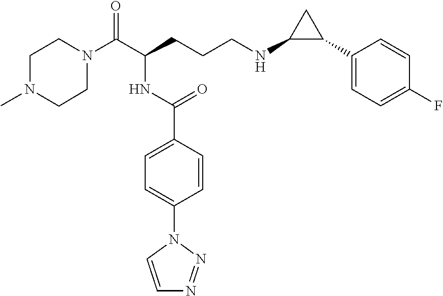 [SMILES: CN1CCN(C(=O)[C@@H](CCCN[C@H]2C[C@@H]2c2ccc(F)cc2)NC(=O)c2ccc(-n3ccnn3)cc2)CC1] |
| 134 | 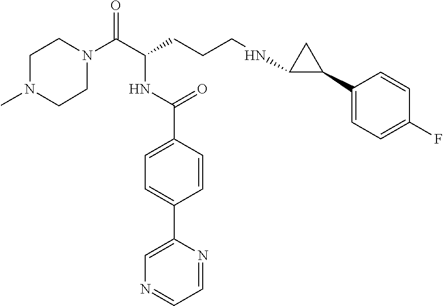 [SMILES: CN1CCN(C(=O)[C@H](CCCN[C@@H]2C[C@H]2c2ccc(F)cc2)NC(=O)c2ccc(-c3cnccn3)cc2)CC1] |
| 135 | 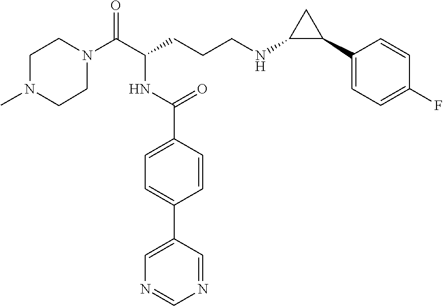 [SMILES: CN1CCN(C(=O)[C@H](CCCN[C@@H]2C[C@H]2c2ccc(F)cc2)NC(=O)c2ccc(-c3cncnc3)cc2)CC1] |
| 136 | 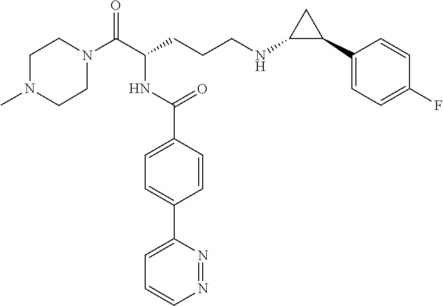 [SMILES: CN1CCN(C(=O)[C@H](CCCN[C@@H]2C[C@H]2c2ccc(F)cc2)NC(=O)c2ccc(-c3cccnn3)cc2)CC1] |
| 137 | 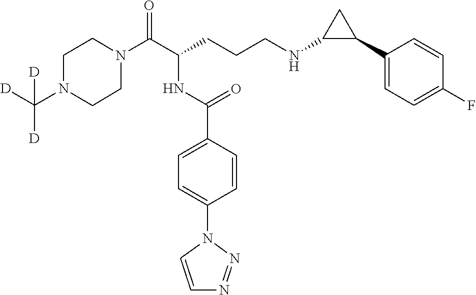 |
| 138 | 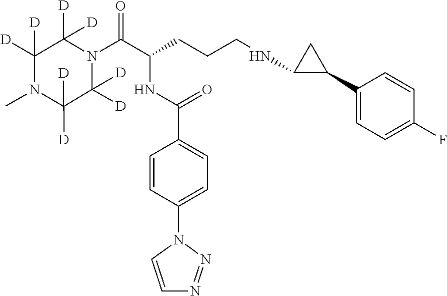 |
| 139 | 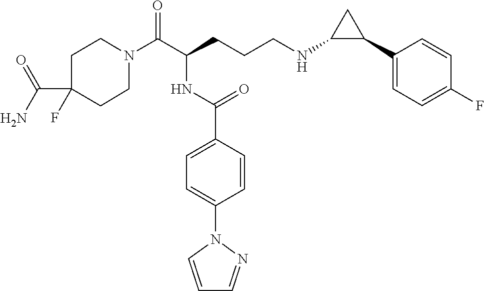 [SMILES: NC(=O)C1(F)CCN(C(=O)[C@@H](CCCN[C@@H]2C[C@H]2c2ccc(F)cc2)NC(=O)c2ccc(-n3cccn3)cc2)CC1] |
| 140 | 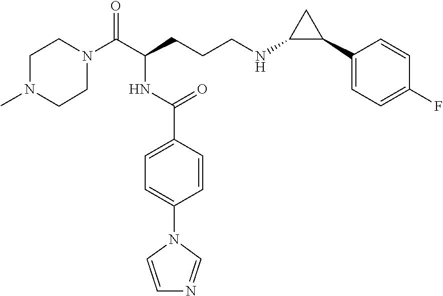 [SMILES: CN1CCN(C(=O)[C@@H](CCCN[C@@H]2C[C@H]2c2ccc(F)cc2)NC(=O)c2ccc(-n3ccnc3)cc2)CC1] |
| 141 | 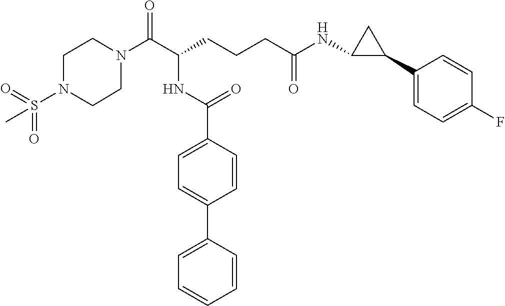 [SMILES: CS(=O)(=O)N1CCN(C(=O)[C@H](CCCC(=O)N[C@@H]2C[C@H]2c2ccc(F)cc2)NC(=O)c2ccc(-c3ccccc3)cc2)CC1] |
| 142 | 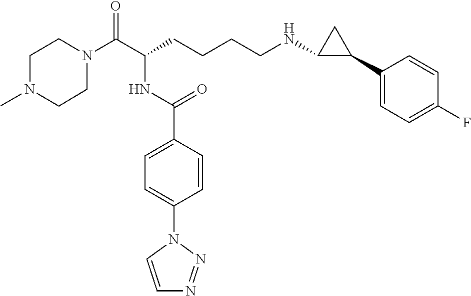 [SMILES: CN1CCN(C(=O)[C@H](CCCCN[C@@H]2C[C@H]2c2ccc(F)cc2)NC(=O)c2ccc(-n3ccnn3)cc2)CC1] |
| 143 | 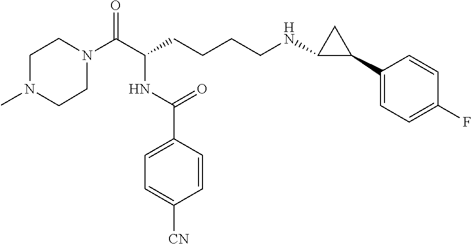 [SMILES: CN1CCN(C(=O)[C@H](CCCCN[C@@H]2C[C@H]2c2ccc(F)cc2)NC(=O)c2ccc(C#N)cc2)CC1] |
| 144 | 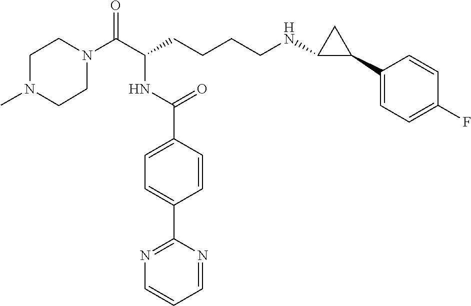 [SMILES: CN1CCN(C(=O)[C@H](CCCCN[C@@H]2C[C@H]2c2ccc(F)cc2)NC(=O)c2ccc(-c3ncccn3)cc2)CC1] |
| 145 | 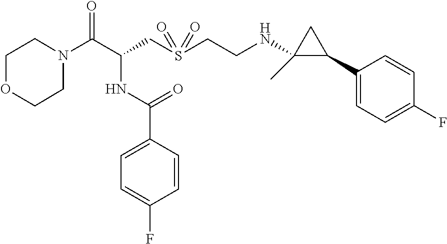 [SMILES: C[C@@]1(NCCS(=O)(=O)C[C@H](NC(=O)c2ccc(F)cc2)C(=O)N2CCOCC2)C[C@H]1c1ccc(F)cc1] |
| 146 | 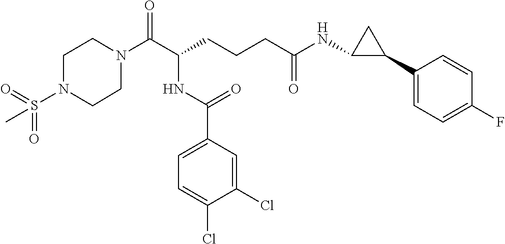 [SMILES: CS(=O)(=O)N1CCN(C(=O)[C@H](CCCC(=O)N[C@@H]2C[C@H]2c2ccc(F)cc2)NC(=O)c2ccc(Cl)c(Cl)c2)CC1] |
| 147 | 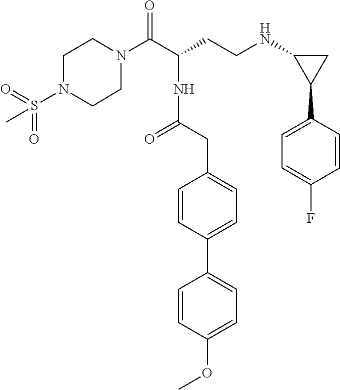 [SMILES: COc1ccc(-c2ccc(CC(=O)N[C@@H](CCN[C@@H]3C[C@H]3c3ccc(F)cc3)C(=O)N3CCN(S(C)(=O)=O)CC3)cc2)cc1] |
| 148 | 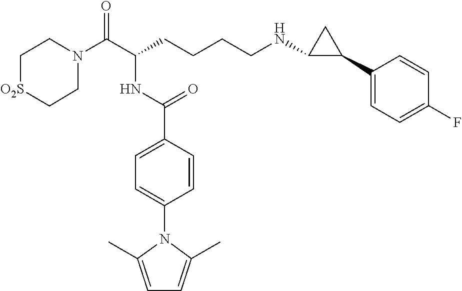 [SMILES: Cc1ccc(C)n1-c1ccc(C(=O)N[C@@H](CCCCN[C@@H]2C[C@H]2c2ccc(F)cc2)C(=O)N2CCS(=O)(=O)CC2)cc1] |
| 149 | 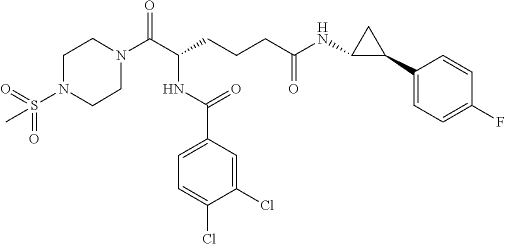 [SMILES: CS(=O)(=O)N1CCN(C(=O)[C@H](CCCC(=O)N[C@@H]2C[C@H]2c2ccc(F)cc2)NC(=O)c2ccc(Cl)c(Cl)c2)CC1] |
| 150 | 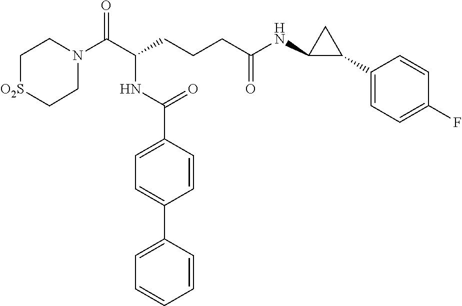 [SMILES: O=C(CCC[C@H](NC(=O)c1ccc(-c2ccccc2)cc1)C(=O)N1CCS(=O)(=O)CC1)N[C@H]1C[C@@H]1c1ccc(F)cc1] |
| 151 | 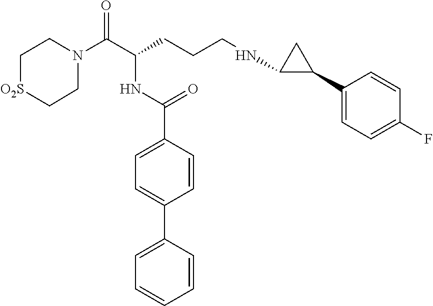 [SMILES: O=C(N[C@@H](CCCN[C@@H]1C[C@H]1c1ccc(F)cc1)C(=O)N1CCS(=O)(=O)CC1)c1ccc(-c2ccccc2)cc1] |
| 152 | 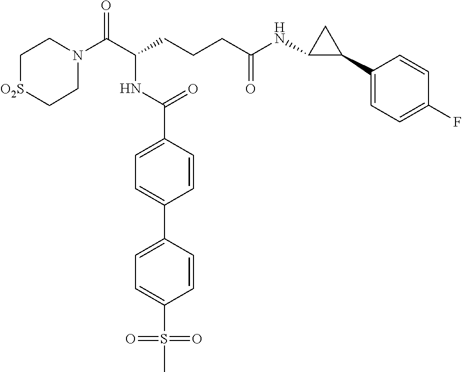 [SMILES: CS(=O)(=O)c1ccc(-c2ccc(C(=O)N[C@@H](CCCC(=O)N[C@@H]3C[C@H]3c3ccc(F)cc3)C(=O)N3CCS(=O)(=O)CC3)cc2)cc1] |
| 153 | 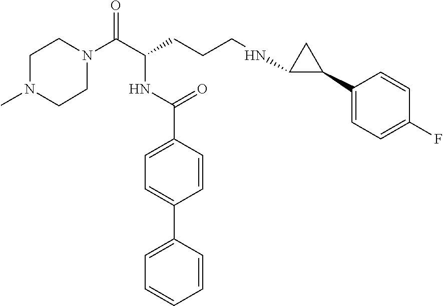 [SMILES: CN1CCN(C(=O)[C@H](CCCN[C@@H]2C[C@H]2c2ccc(F)cc2)NC(=O)c2ccc(-c3ccccc3)cc2)CC1] |
| 154 | 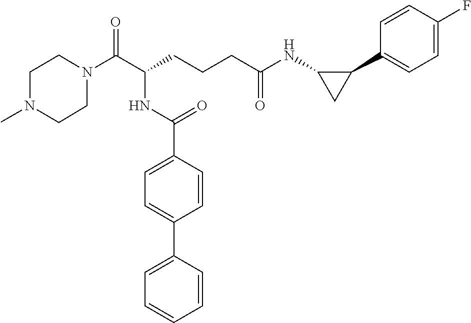 [SMILES: CN1CCN(C(=O)[C@H](CCCC(=O)N[C@H]2C[C@@H]2c2ccc(F)cc2)NC(=O)c2ccc(-c3ccccc3)cc2)CC1] |
| 155 | 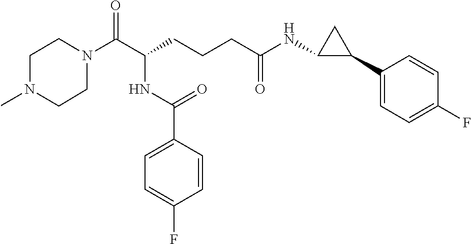 [SMILES: CN1CCN(C(=O)[C@H](CCCC(=O)N[C@@H]2C[C@H]2c2ccc(F)cc2)NC(=O)c2ccc(F)cc2)CC1] |
| 156 | 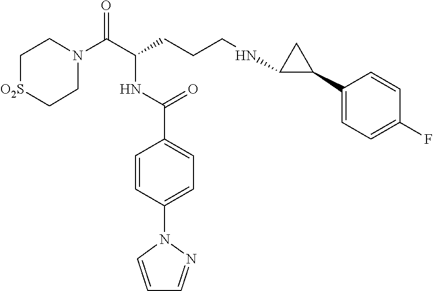 [SMILES: O=C(N[C@@H](CCCN[C@@H]1C[C@H]1c1ccc(F)cc1)C(=O)N1CCS(=O)(=O)CC1)c1ccc(-n2cccn2)cc1] |
| 157 | 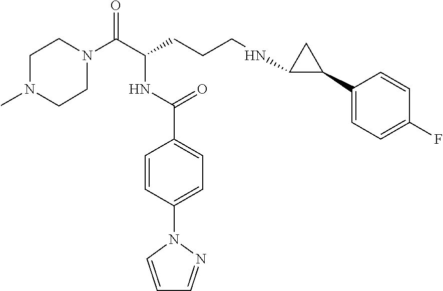 [SMILES: CN1CCN(C(=O)[C@H](CCCN[C@@H]2C[C@H]2c2ccc(F)cc2)NC(=O)c2ccc(-n3cccn3)cc2)CC1] |
| 158 | 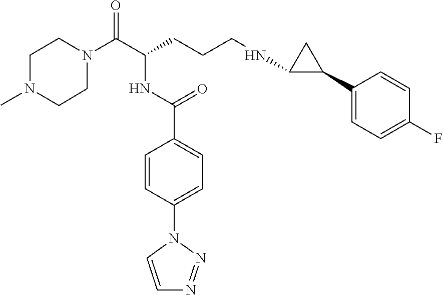 [SMILES: CN1CCN(C(=O)[C@H](CCCN[C@@H]2C[C@H]2c2ccc(F)cc2)NC(=O)c2ccc(-n3ccnn3)cc2)CC1] |
| 159 | 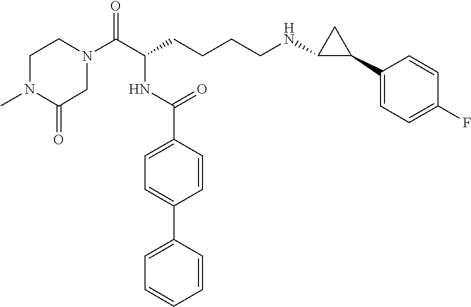 [SMILES: CN1CCN(C(=O)[C@H](CCCCN[C@@H]2C[C@H]2c2ccc(F)cc2)NC(=O)c2ccc(-c3ccccc3)cc2)CC1=O] |
| 160 | 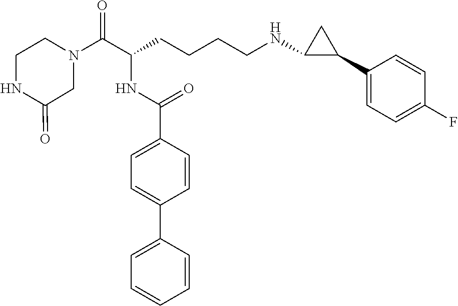 [SMILES: O=C1CN(C(=O)[C@H](CCCCN[C@@H]2C[C@H]2c2ccc(F)cc2)NC(=O)c2ccc(-c3ccccc3)cc2)CCN1] |
| 161 | 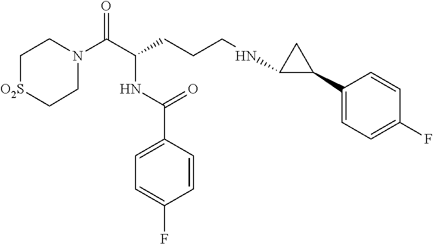 [SMILES: O=C(N[C@@H](CCCN[C@@H]1C[C@H]1c1ccc(F)cc1)C(=O)N1CCS(=O)(=O)CC1)c1ccc(F)cc1] |
| 162 | 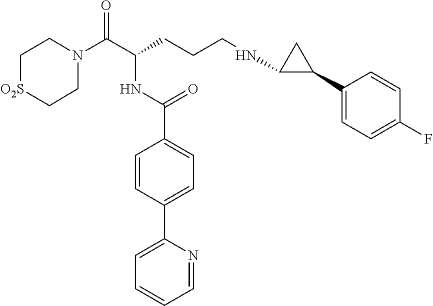 [SMILES: O=C(N[C@@H](CCCN[C@@H]1C[C@H]1c1ccc(F)cc1)C(=O)N1CCS(=O)(=O)CC1)c1ccc(-c2ccccn2)cc1] |
| 163 | 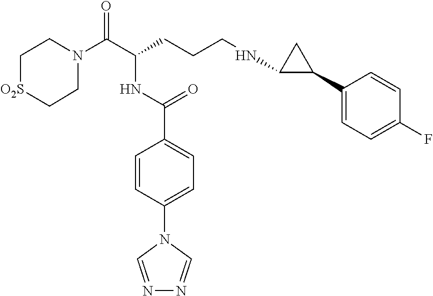 [SMILES: O=C(N[C@@H](CCCN[C@@H]1C[C@H]1c1ccc(F)cc1)C(=O)N1CCS(=O)(=O)CC1)c1ccc(-n2cnnc2)cc1] |
| 164 | 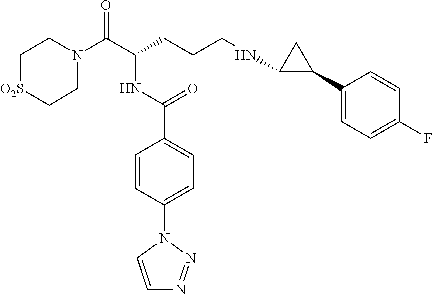 [SMILES: O=C(N[C@@H](CCCN[C@@H]1C[C@H]1c1ccc(F)cc1)C(=O)N1CCS(=O)(=O)CC1)c1ccc(-n2ccnn2)cc1] |
| 165 | 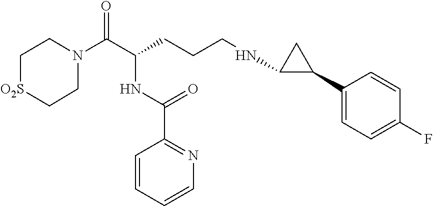 [SMILES: O=C(N[C@@H](CCCN[C@@H]1C[C@H]1c1ccc(F)cc1)C(=O)N1CCS(=O)(=O)CC1)c1ccccn1] |
| 166 | 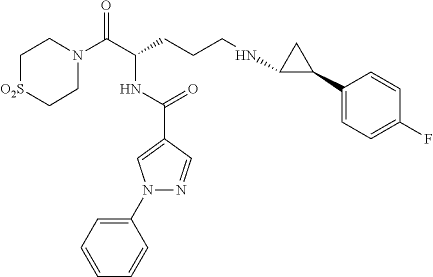 [SMILES: O=C(N[C@@H](CCCN[C@@H]1C[C@H]1c1ccc(F)cc1)C(=O)N1CCS(=O)(=O)CC1)c1cnn(-c2ccccc2)c1] |
| 167 | 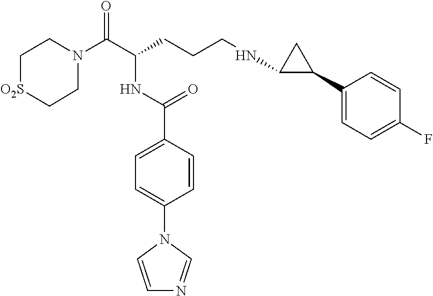 [SMILES: O=C(N[C@@H](CCCN[C@@H]1C[C@H]1c1ccc(F)cc1)C(=O)N1CCS(=O)(=O)CC1)c1ccc(-n2ccnc2)cc1] |
| 168 | 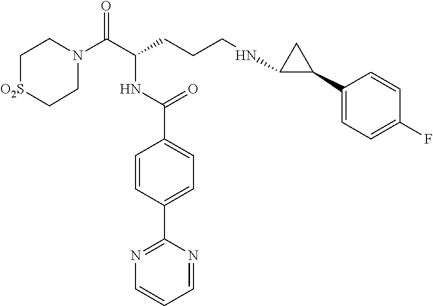 [SMILES: O=C(N[C@@H](CCCN[C@@H]1C[C@H]1c1ccc(F)cc1)C(=O)N1CCS(=O)(=O)CC1)c1ccc(-c2ncccn2)cc1] |
| 169 | 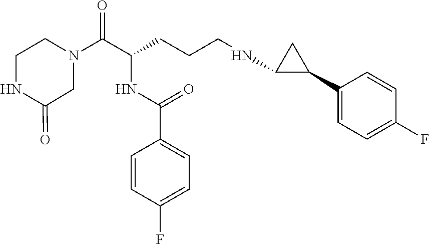 [SMILES: O=C1CN(C(=O)[C@H](CCCN[C@@H]2C[C@H]2c2ccc(F)cc2)NC(=O)c2ccc(F)cc2)CCN1] |
| 170 | 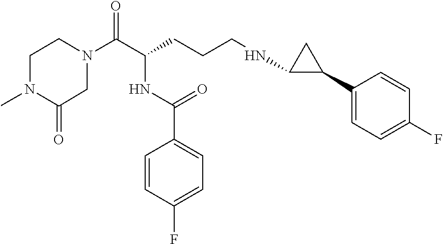 [SMILES: CN1CCN(C(=O)[C@H](CCCN[C@@H]2C[C@H]2c2ccc(F)cc2)NC(=O)c2ccc(F)cc2)CC1=O] |
| 171 | 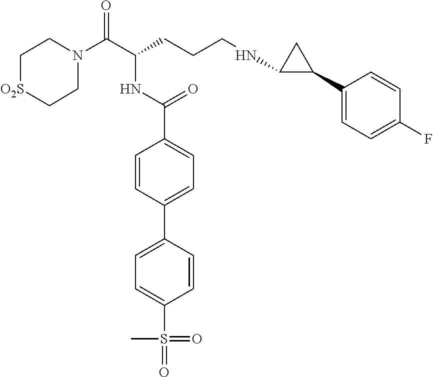 [SMILES: CS(=O)(=O)c1ccc(-c2ccc(C(=O)N[C@@H](CCCN[C@@H]3C[C@H]3c3ccc(F)cc3)C(=O)N3CCS(=O)(=O)CC3)cc2)cc1] |
| 172 | 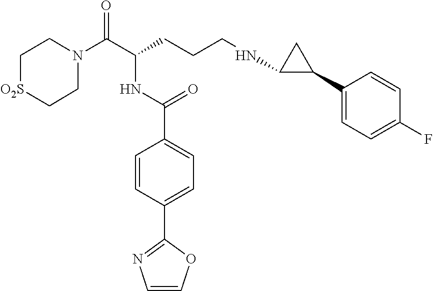 [SMILES: O=C(N[C@@H](CCCN[C@@H]1C[C@H]1c1ccc(F)cc1)C(=O)N1CCS(=O)(=O)CC1)c1ccc(-c2ncco2)cc1] |
| 173 | 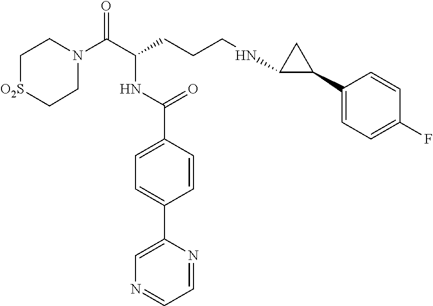 [SMILES: O=C(N[C@@H](CCCN[C@@H]1C[C@H]1c1ccc(F)cc1)C(=O)N1CCS(=O)(=O)CC1)c1ccc(-c2cnccn2)cc1] |
| 174 | 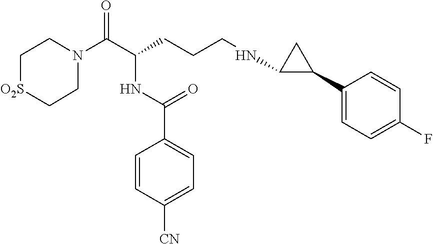 [SMILES: N#Cc1ccc(C(=O)N[C@@H](CCCN[C@@H]2C[C@H]2c2ccc(F)cc2)C(=O)N2CCS(=O)(=O)CC2)cc1] |
| 175 | 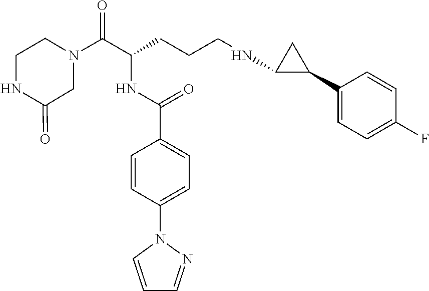 [SMILES: O=C1CN(C(=O)[C@H](CCCN[C@@H]2C[C@H]2c2ccc(F)cc2)NC(=O)c2ccc(-n3cccn3)cc2)CCN1] |
| 176 | 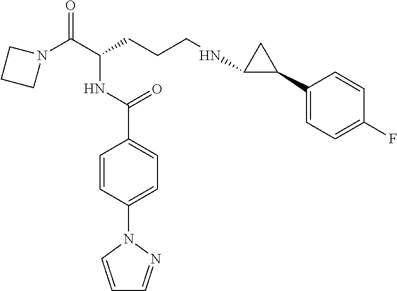 [SMILES: O=C(N[C@@H](CCCN[C@@H]1C[C@H]1c1ccc(F)cc1)C(=O)N1CCC1)c1ccc(-n2cccn2)cc1] |
| 177 | 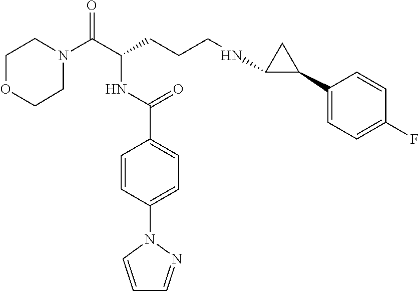 [SMILES: O=C(N[C@@H](CCCN[C@@H]1C[C@H]1c1ccc(F)cc1)C(=O)N1CCOCC1)c1ccc(-n2cccn2)cc1] |
| 178 | 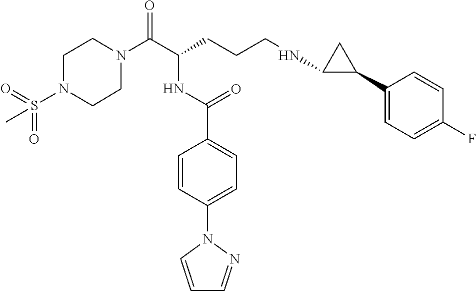 [SMILES: CS(=O)(=O)N1CCN(C(=O)[C@H](CCCN[C@@H]2C[C@H]2c2ccc(F)cc2)NC(=O)c2ccc(-n3cccn3)cc2)CC1] |
| 179 | 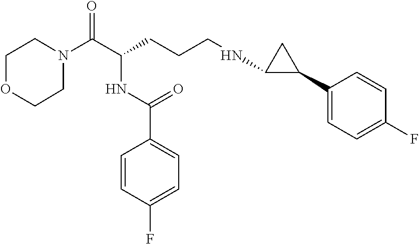 [SMILES: O=C(N[C@@H](CCCN[C@@H]1C[C@H]1c1ccc(F)cc1)C(=O)N1CCOCC1)c1ccc(F)cc1] |
| 180 | 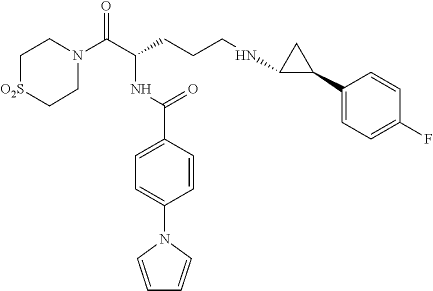 [SMILES: O=C(N[C@@H](CCCN[C@@H]1C[C@H]1c1ccc(F)cc1)C(=O)N1CCS(=O)(=O)CC1)c1ccc(-n2cccc2)cc1] |
| 181 | 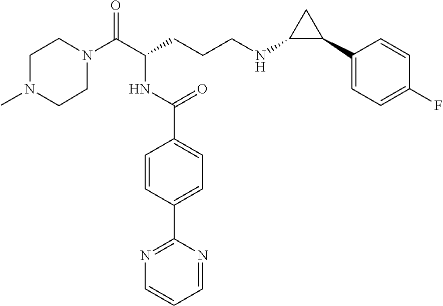 [SMILES: CN1CCN(C(=O)[C@H](CCCN[C@@H]2C[C@H]2c2ccc(F)cc2)NC(=O)c2ccc(-c3ncccn3)cc2)CC1] |
| 182 | 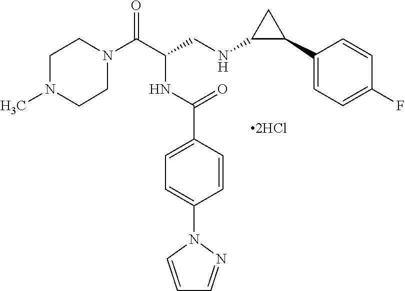 |
| 183 | 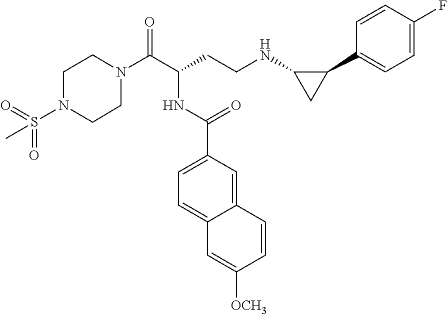 [SMILES: COc1ccc2cc(C(=O)N[C@@H](CCN[C@H]3C[C@@H]3c3ccc(F)cc3)C(=O)N3CCN(S(C)(=O)=O)CC3)ccc2c1] |
| 184 | 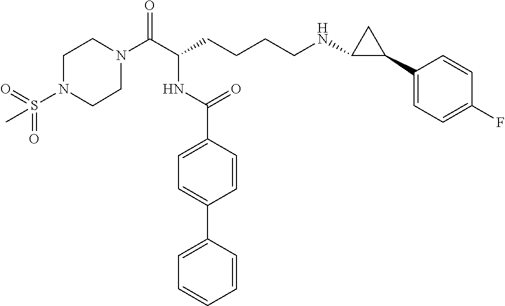 [SMILES: CS(=O)(=O)N1CCN(C(=O)[C@H](CCCCN[C@@H]2C[C@H]2c2ccc(F)cc2)NC(=O)c2ccc(-c3ccccc3)cc2)CC1] |
| 185 | 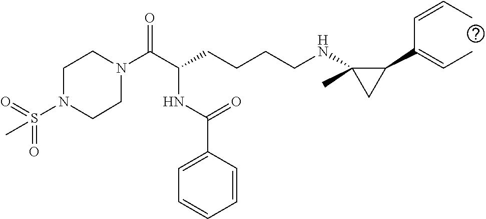 [SMILES: C/C=C\C(=C/C)[C@H]1C[C@]1(C)NCCCC[C@H](NC(=O)c1ccccc1)C(=O)N1CCN(S(C)(=O)=O)CC1] |
| 186 | 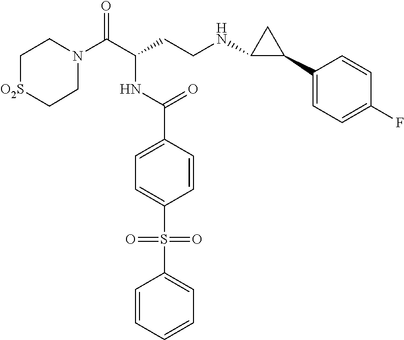 [SMILES: O=C(N[C@@H](CCN[C@@H]1C[C@H]1c1ccc(F)cc1)C(=O)N1CCS(=O)(=O)CC1)c1ccc(S(=O)(=O)c2ccccc2)cc1] |
| 187 | 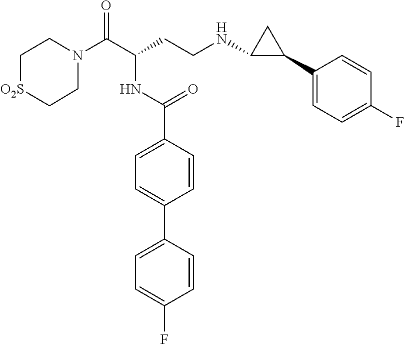 [SMILES: O=C(N[C@@H](CCN[C@@H]1C[C@H]1c1ccc(F)cc1)C(=O)N1CCS(=O)(=O)CC1)c1ccc(-c2ccc(F)cc2)cc1] |
| 188 | 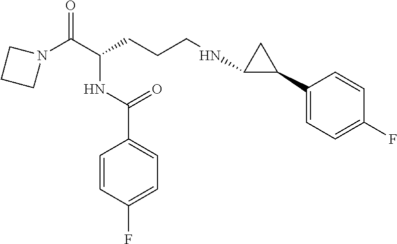 [SMILES: O=C(N[C@@H](CCCN[C@@H]1C[C@H]1c1ccc(F)cc1)C(=O)N1CCC1)c1ccc(F)cc1] |

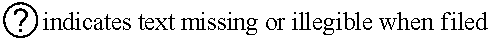

### General Synthetic Methods for Preparing Compounds

In the Examples below and throughout the disclosure, the following abbreviations may be used: PTFE=polytetrafluoroethylene; RM=Reaction Mixture; R H=Relative Humidity; RT=Room Temperature; SM=Starting Material; MeCN=acetonitrile; CIPh=chlorophenol; DCE=dichloroethane; DCM=dichloromethane; DIPE=di-isopropylether; DMA=dimethyl acetamide; DMF=dimethyl formamide; DMSO=dimethylsulfoxide; Et₂O=di-ethyl ether; EtOAc=ethyl acetate; EtOH=ethanol; H₂O=water; I PA=propan-2-ol; i-PrOAc=iso-propyl acetate; MEK=methyl ethyl ketone; MeOH=methanol; MIBK=methyl isobutyl ketone; MTBE=methyl tert-butyl ether; n-BuOAc=n-butyl acetate; n-BuOH=n-butanol; NMP=n-methyl pyrrolidone; n-PrOH=n-propanol; s-BuOAc=s-butyl acetate; t-BuOH=t-butanol; TFA=tri-fluoro acetic acid; THF=tetrahydrofuran; TMP=2,2,4-trimethylpentane; ¹H-NMR=Proton Nuclear magnetic Resonance; DSC=Differential Scanning Calorimetry; DVS=Dynamic Vapour Sorption; GVS=Gravimetric Vapour Sorption; HPLC=High Performance Liquid Chromatography; HS=Head Space; HSM=Hot Stage Microscopy; IC=Ion Chromatography; IDR=Intrinsic Dissolution Rate; KF=Karl-Fisher; MAS=Magic Angle Spinning; MDSC=Modulated Differential Scanning Calorimetry; PLM=Polarised Light Microscopy; PVM=Particle Vision and Measurement; SCXRD=Single Crystal X-Ray Diffraction; SS-NMR=Solid State Nuclear Magnetic Resonance; TGA=Thermal Gravimetric Analysis; UV=UltraViolet VH-XRPD=Variable Humidity X-Ray Powder Diffraction; VT-XRPD=Variable Temperature X-Ray Powder Diffraction; and XRPD=X-Ray Powder Diffraction. Other abbreviations may be used and will be familiar in context to those of skill in the art.

The invention is further illustrated by the following non-limiting examples. The methods exemplified below may also be extrapolated to compounds disclosed herein. Further methods suitable for use in preparation of examples of the present invention may be found in WO 2015/021128 and WO 2016/130952, the contents of which are hereby incorporated by reference as if written herein in their entireties. Additional LSD1 inhibitors may be prepared by methods disclosed above in paragraph 101761.

### Intermediate A: (1R,2S)-2-(4-fluorophenyl)-1-methylcyclopropanamine

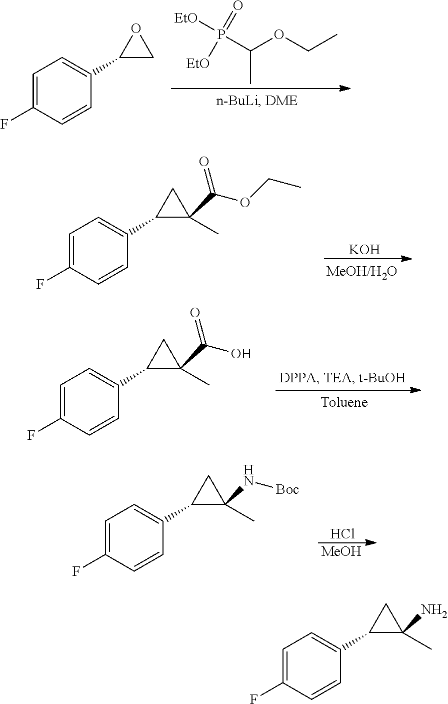

A solution of ethyl 2-(diethoxyphosphoryl)propanoate (3.45 g, 14.48 mmol, 2.00 equiv) in ethylene glycol dimethyl ether (20 mL) was treated with n-BuLi (2.5M) (5.8 mL) dropwise with stirring at 0° C. The resulting solution was stirred for 30 min at room temperature. To this was added 2-(4-fluorophenyl)oxirane (1 g, 7.24 mmol, 1.00 equiv). The resulting solution was stirred for 12 h while the temperature was maintained at 80° C. in an oil bath. The reaction mixture was cooled to RT. The reaction was then quenched by the addition of 20 mL of water. The resulting solution was extracted with ethyl acetate and the organic layers was dried and concentrated. The residue was chromatographed on silica gel and eluted with ethyl acetate/petroleum ether (1:100). This resulted in 1 g (62%) of ethyl (1R)-2-(4-fluorophenyl)-1-methylcyclopropane-1-carboxylate as yellow oil. A solution of ethyl (1R)-2-(4-fluorophenyl)-1-methylcyclopropane-1-carboxylate (1 g, 4.50 mmol, 1.00 equiv) in methanol/H₂O (10/2 mL) and potassium hydroxide (1.26 g, 22.46 mmol, 4.99 equiv) was stirred for 10 h at room temperature. The resulting solution was diluted with H₂O. The pH value of the solution was adjusted to 2 with hydrochloric acid (2 mol/L). The resulting solution was extracted with ethyl acetate and the organic layers combined and dried over anhydrous sodium sulfate and concentrated under vacuum. This resulted in 800 mg (92%) of (1R)-2-(4-fluorophenyl)-1-methylcyclopropane-1-carboxylic acid as yellow oil. A solution of (1R)-2-(4-fluorophenyl)-1-methylcyclopropane-1-carboxylic acid (400 mg, 2.06 mmol, 1.00 equiv) in toluene (10 mL) was mixed with diphenoxyphosphoryl azide (680 mg, 2.47 mmol, 1.20 equiv), and triethylamine (312 mg, 3.08 mmol, 1.50 equiv). The resulting solution was stirred for 30 min at 90° C. in an oil bath. Then, tert-butanol (2 mL) was added. The resulting solution was allowed to react, with stirring, for an additional 12 h while the temperature was maintained at 90° C. in an oil bath. The reaction mixture was cooled to room temperature and the resulting solution was diluted with ethyl acetate. The resulting mixture was washed with H₂O. The mixture was dried over anhydrous sodium sulfate and concentrated under vacuum. The residue was chromatographed on a silica gel column and eluted with ethyl acetate/petroleum ether (1:100). This resulted in 350 mg (64%) of tert-butyl N-[(1R)-2-(4-fluorophenyl)-1-methylcyclopropyl]carbamate as yellow oil. A solution of tert-butyl N-[(1R,2S)-2-(4-fluorophenyl)-1-methylcyclopropyl]carbamate (350 mg, 1.32 mmol, 1.00 equiv) in methanol (HCl) (10 mL) was stirred for 2 h at room temperature. The resulting solution was diluted with 10 mL of H₂O. The pH value of the solution was adjusted to 9 with saturated sodium bicarbonatesolution. The resulting solution was extracted with 3×10 mL of ethyl acetate and the organic layers combined and dried over anhydrous sodium sulfate and concentrated under vacuum. This resulted in 200 mg (92%) of (1R,2S)-2-(4-fluorophenyl)-1-methylcyclopropan-1-amine as yellow oil.

### Example A1: N—((S)-1-oxo-6-(((1R,2S)-2-phenylcyclopropyl)amino)-1-(pyrrolidin-1-yl)hexan-2-yl)benzamide

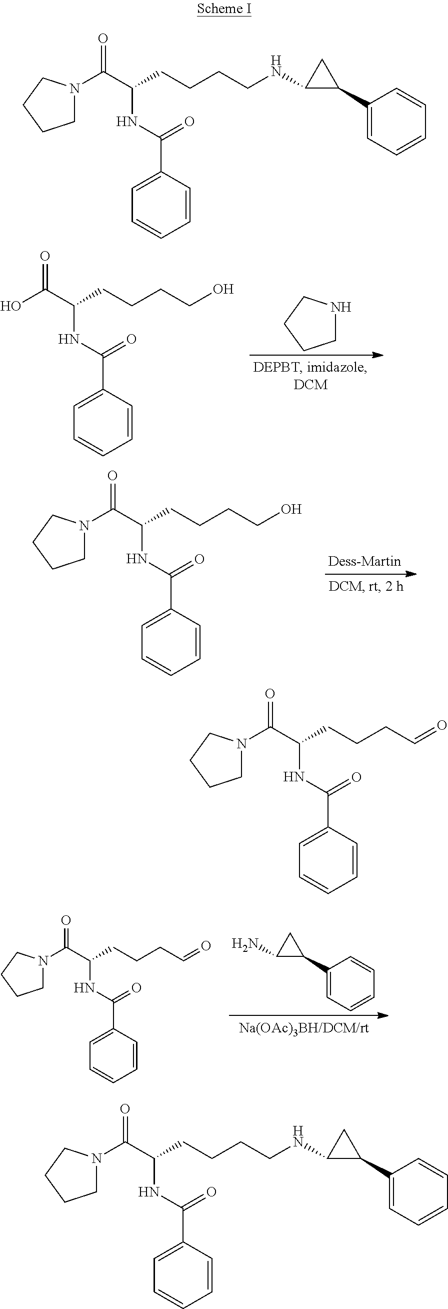

(S)-2-benzamido-6-hydroxyhexanoic acid was prepared from (S)-2-amino-6-hydroxyhexanoic acid. This material (1 g, 3.98 mmol, 1.00 equiv) in tetrahydrofuran was reacted with 3-(diethoxyphosphoryloxy)-1,2,3-benzotriazin-4(3H)-one (DEPBT) (2.4 g, 8.03 mmol, 2.00 equiv) and imidazole (542 mg, 7.97 mmol, 2.00 equiv). This was followed by the addition of a solution of pyrrolidine (283 mg, 3.98 mmol, 1.00 equiv) in tetrahydrofuran at 0° C. in 30 min. The resulting solution was stirred for 16 h at room temperature. The solution was diluted with KH₂PO₄ (aq.). The aqueous layer was extracted with ethyl acetate and the organic layers were washed with brine and dried over anhydrous sodium sulfate. After filtration, solvent was removed under reduced pressure. The residue was purified by preparative HPLC and eluted with MeCN with 0.5% NH₄HCO₃. This resulted in 640 mg (53%) of (S)—N-(6-hydroxy-1-oxo-1-(pyrrolidin-1-yl)hexan-2-yl)benzamide as a light yellow oil. (S)—N-(6-hydroxy-1-oxo-1-(pyrrolidin-1-yl)hexan-2-yl)benzamide (640 mg, 2.10 mmol, 1.00 equiv) in dichloromethane (100 ml) was oxidized with Dess-Martin periodinane (DMP) (893 mg, 2.11 mmol, 1.00 equiv). The resulting solution was stirred for 30 min at 0° C. in a water/ice bath and was then diluted with Na₂SO₃ (aq.) and NaHCO₃ (aq.). The aqueous layers were extracted with ethyl acetate and the organic layers were washed with brine and dried over anhydrous sodium sulfate. After filtration, solvent was removed under reduced pressure. The residue was chromatographed on silica gel and eluted with ethyl acetate/petroleum ether (10:1). This gave 150 mg (24%) of (S)—N-(1,6-dioxo-1-(pyrrolidin-1-yl)hexan-2-yl)benzamide as a white solid. (S)—N-(1,6-dioxo-1-(pyrrolidin-1-yl)hexan-2-yl)benzamide (150 mg, 0.50 mmol, 1.00 equiv) was dissolved in dichloromethane (25 mL). (1R,2S)-2-phenylcyclopropanamine (66 mg, 0.50 mmol, 1.00 equiv) was added. After stirring 5 minutes, sodium triacetoxyborohydride (252 mg, 1.19 mmol, 2.40 equiv) was added. The resulting solution was stirred for 30 min at 0° C. After the reaction was completed, the resulting solution was diluted with sat.NaHCO₃. Then it was extracted with dichloromethane. The organic layers were washed with brine and dried over anhydrous sodium sulfate. Solvent was removed under reduced pressure and the residue was purified by Prep-HPLC (CAN/H₂O with 0.5% NH₄HCO₃). This resulted in 29 mg (14%) of N—((S)-1-oxo-6-(((1R,2S)-2-phenvlcyclopropyl)amino)-1-(pyrrolidin-1-yl)hexan-2-yl)benzamide as colorless oil. ¹H NMR (300 MHz, CD₃OD-d₄) δ ppm: 7.85 (d, J=7.5 Hz, 2H), 7.60-7.00 (m, 8H), 4.85-4.75 (m, 1H), 3.92-3.80 (m, 1H), 3.70-3.30 (m, 4H), 2.74 (t, J=7.2 Hz, 1H), 2.36-2.28 (m, 1H), 2.07-1.75 (m, 7H), 1.74-1.37 (m, 4H), 1.10-0.95 (m, 2H); MS (ES, m/z): 420 (M+H).

### Example A2: N—((S)-1-oxo-6-(((1R,2S)-2-phenylcyclopropyl)amino)-1-(piperidin-1-yl)hexan-2-yl)benzamide

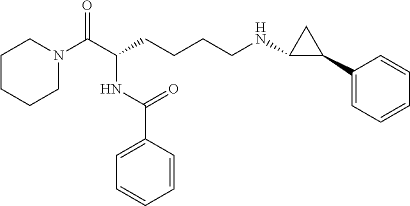 [SMILES: O=C(N[C@@H](CCCCN[C@@H]1C[C@H]1c1ccccc1)C(=O)N1CCCCC1)c1ccccc1]

N—((S)-1-oxo-6-(((1R,2S)-2-phenylcyclopropyl)amino)-1-(piperidin-1-yl)hexan-2-yl)benzamide was prepared in the same manner as was described for the synthesis of N—((S)-1-oxo-6-(((1R,2S)-2-phenylcyclopropyl)amino)-1-(pyrrolidin-1-yl)hexan-2-yl)benzamide. (S)-2-benzamido-6-hydroxyhexanoic acid was coupled with piperidine using 3-(diethoxyphosphoryloxy)-1,2,3-benzotriazin-4(3H)-one and imidazole. The resultant alcohol (S)—N-(6-hydroxy-1-oxo-1-(piperidin-1-yl)hexan-2-yl)benzamide was oxidized under Dess-Martin conditions to the aldehyde (S)—N-(1,6-dioxo-1-(piperidin-1-yl)hexan-2-yl)benzamide. This was coupled with (I R,2S)-2-phenylcyclopropanamine under reductive amination conditions (Na(OAc)₃BH) to yield the desired product N—((S)-1-oxo-6-(((1R,2S)-2-phenylcyclopropyl)amino)-1-(piperidin-1-yl)hexan-2-yl)benzamide as a colorless oil. ES, m/z=434 (M+H). ¹H NMR (300 MHz, CD₃OD-d₄) δ ppm: 7.86 (d, J=7.2 Hz, 2H), 7.70-7.40 (m, 3H), 7.30-7.15 (m, 2H), 7.15-7.08 (m, 1H), 7.06 (d, J=7.2 Hz, 2H), 5.15-5.00 (m, 1H), 3.80-3.60 (m, 2H), 3.60-3.40 (m, 2H), 2.34 (t, J=7.2 Hz, 2H), 2.40-2.30 (m, 1H), 2.10-1.40 (m, 4H), 1.15-1.00 (m, 2H).

### Example A3: 4-fluoro-N—((S)-6-(((1R,2S)-2-(4-fluorophenyl)cyclopropyl)amino)-1-(4-methylpiperazin-1-yl)-1-oxohexan-2-yl)benzamide (Compound 1)

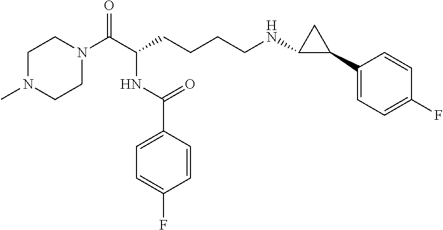 [SMILES: CN1CCN(C(=O)[C@H](CCCCN[C@@H]2C[C@H]2c2ccc(F)cc2)NC(=O)c2ccc(F)cc2)CC1]

4-fluoro-N—((S)-6-(((I R,2S)-2-(4-fluorophenyl)cyclopropyl)amino)-1-(4-methylpiperazin-1-yl)-1-oxohexan-2-yl)benzamide was prepared in a manner analogous to Example A2. The alcohol 4-fluoro-N—((S)-6-(((1R,2S)-2-(4-fluorophenyl)cyclopropyl)amino)-1-(4-methylpiperazin-1-yl)-1-oxohexan-2-yl)benzamide was prepared by reduction of (S)-2-(4-fluorobenzamido)hexanedioic acid with Me₂S—BH₃. This type of reduction was used to prepare similar alcohols (e.g. The alcohol starting material (S)-2-benzamido-6-hydroxyhexanoic acid for the synthesis of N—((S)-1-oxo-6-(((1R,2S)-2-phenylcyclopropyl)amino)-1-(pyrrolidin-1-yl)hexan-2-yl)benzamide (Example A1)). Into a 1000-mL 3-necked round-bottom flask purged and maintained with an inert atmosphere of nitrogen, was placed a solution of (S)-2-(4-fluorobenzamido)hexanedioic acid (10 g, 35.30 mmol, 1.00 equiv) in tetrahydrofuran (300 ml). Then a solution of Me₂S—BH₃ (11 mL, 3.00 equiv) in tetrahydrofuran (50 ml) was added at 0° C. The resulting solution was stirred for 3 h at 0° C. in an ice/salt bath. The reaction was then quenched by the addition of 20 ml of methanol. The resulting mixture was concentrated under vacuum. The resulting solution was diluted with 300 ml of sat.Na₂CO₃. The resulting solution was extracted with 3×100 mL of ethyl acetate and the aqueous layers combined. The pH value of the solution was adjusted to 2 with hydrochloric acid (2 mol/L). The resulting solution was extracted with 3×200 ML of ethyl acetate and the organic layers combined. The resulting mixture was washed with 1×500 mL of brine. The mixture was dried over anhydrous sodium sulfate. The solids were filtered out. The resulting mixture was concentrated under vacuum. This resulted in 6 g (63%) of(S)-2-(4-fluorobenzamido)-6-hydroxyhexanoic acid as colorless oil. This material was reacted with N-methyl piperazine followed by Dess-Martin oxidation and coupling via reductive amination with (1R,2S)-2-(4-fluorophenyl)cyclopropanamine in the manner described for the synthesis of N—((S)-1-oxo-6-(((1R,2S)-2-phenylcyclopropyl)amino)-1-(pyrrolidin-1-yl)hexan-2-yl)benzamide (Example A1) to yield the desired product 4-fluoro-N—((S)-6-(((1R,2S)-2-(4-fluorophenyl)cyclopropyl)amino)-1-(4-methylpiperazin-1-yl)-1-oxohexan-2-yl)benzamide as colorless oil. ES, m/s=485*M+H). ¹H NMR (300 MHz, CD₃OD-d₄) δ ppm: 7.83 (dd, J₁=5.4 Hz, J=1.4 Hz, 2H), 7.18-7.04 (m, 3H), 7.00-6.87 (m, 4H), 5.17-5.05 (m 1H), 3.78-3.50 (m, 4H), 2.71 (t. J=6.9 Hz, 2H), 2.30 (s, 3H), 2.28-2.21 (m, 1H), 1.90-1.78 (m, 2H), 1.72-1.31 (m, 9H), 1.07-0.96 (m, 1H), 0.94-0.86 (m, 1H).

### Example 158: N-[(2S)-1-(4-(methyl)piperazin-1-yl)-5-[[(1R,2S)-2-(4-fluorophenyl)cyclopropyl]amino]-1-oxopentan-2-yl]-4-(1H-1,2,3-triazol-1-yl)benzamide

(Compound 2) was prepared according to the method of Scheme II.

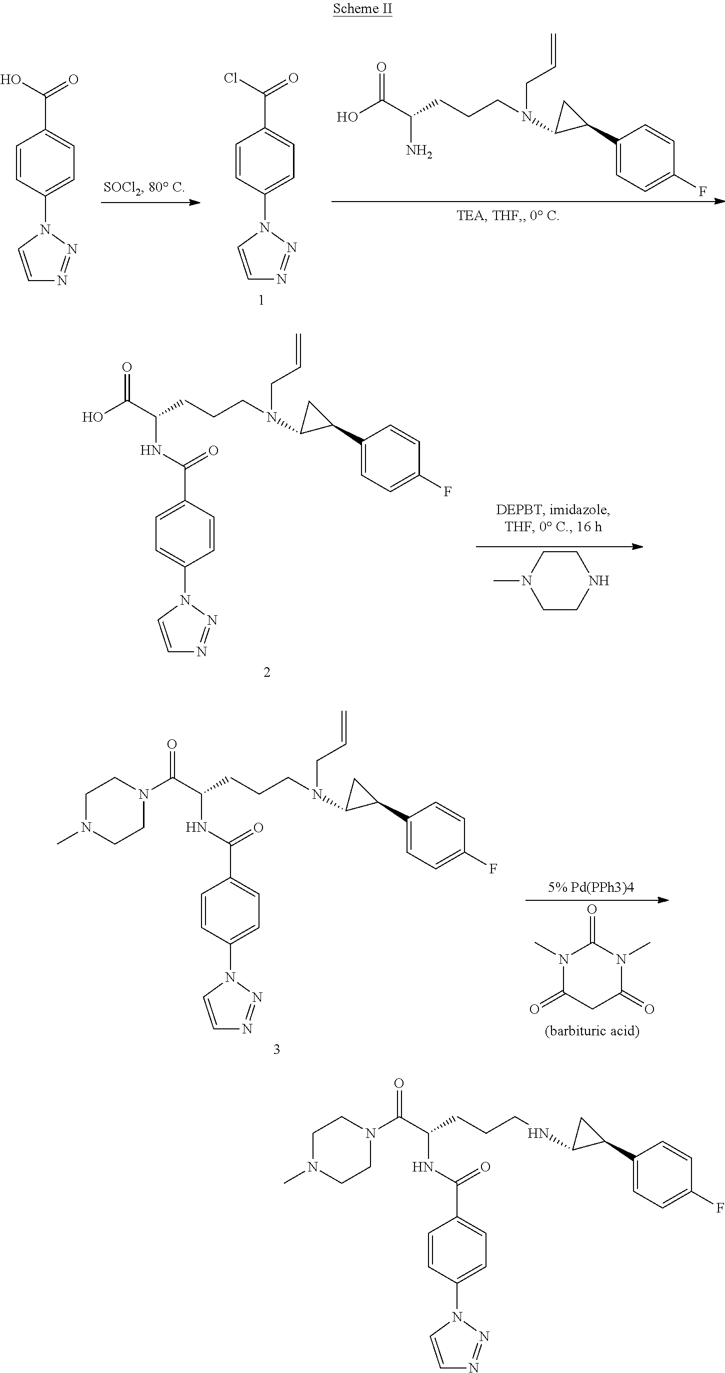

### 4-(1H-1,2,3-triazolyl-1-yl)benzoyl Chloride (1)

In a 100-mL round-bottom flask were combined 4-(1H-1,2,3-triazol-1-yl)benzoic acid (1 g, 5.29 mmol, 1.00 equiv) and thionyl chloride (20 mL). The resulting solution was stirred for 16 h at 80° C. in an oil bath. The resulting mixture was then concentrated under reduced pressure, affording 1 g (91%) of intermediate (1) as a yellow solid.

### (2S)-5-[[(1R,2S)-2-(4-fluorophenyl)cyclopropyl](propen-3-yl)amino]-2-[[4-(1H-1,2,3-triazol-1-yl)phenyl]formamido]pentanoic Acid (2)

In a 100-mL round-bottom flask were combined (2S)-2-amino-5-[(1R,2S)-2-(4-fluorophenyl)cyclopropyl](prop-2-en-1-yl)aminopentanoic acid (500 mg, 1.63 mmol, 1.00 equiv). Et₃N (494 mg, 4.88 mmol, 3.00 equiv) and THF (20 mL). This was followed by the addition of a solution of intermediate (1) from the previous step (1 g, 4.82 mmol, 2.95 equiv) in THF (20 mL) dropwise with stirring at 0° C. in 30 min. The resulting solution was stirred for 1 h at 0° C. in an ice/salt bath, then concentrated under reduced pressure, and applied onto a silica gel column with CH₂C₂/methanol (10:1). The collected fractions were combined and concentrated under reduced pressure, affording 400 mg (51%) of intermediate (2) as a off-white solid.

### N-[(2S)-1-(4-(methyl)piperazin-1-yl)-5-[[(1R,2S)-2-(4-fluorophenyl)-cyclopropyl](prop-2-en-1-yl)amino]-1-oxopentan-2-yl]-4-(1H-1,2,3-triazol-1-yl)-benzamide (3)

In a 100-mL round-bottom flask were combined intermediate (2) from the previous step (400 mg, 0.84 mmol, 1.00 equiv), DEPBT (375 mg, 1.25 mmol, 1.50 equiv), and THF (20 mL), followed by the addition of imidazole (85 mg, 1.25 mmol, 1.50 equiv). The mixture was stirred for 30 min at 0° C. at which point 1-methylpiperazine (127 mg, 1.27 mmol, 1.50 equiv) was added dropwise with stirring at 0° C. in 3 min. The resulting solution was stirred for 16 h at 20° C., then concentrated under reduced pressure. The residue was applied onto a silica gel column with CHCl₂/methanol (10:1). The collected fractions were combined and concentrated under vacuum, affordin 300 mg (64%) of intermediate (3) as a yellow solid.

### N-[(2S)-1-(4-(methyl)piperazin-1-yl)-5-[[(1R,2S)-2-(4-fluorophenyl)-cyclopropyl]amino]-1-oxopentan-2-yl]-4-(1H-1,2,3-triazol-1-yl)benzamide (Example 158; Compound 2)

Into a 100-mL round-bottom flask purged and maintained with an inert atmosphere of nitrogen, was placed N-[(2S)-5-[[(1R,2S)-2-(4-fluorophenyl)cyclopropyl](prop-2-en-1-yl)amino]-1-(4-methylpiperazin-1-yl)-1-oxopentan-2-yl]-4-(1H-1,2,3-triazol-1-yl)benzamide (300 mg, 0.54 mmol, 1.00 equiv), 1,3-dimethyl-1,3-diazinane-2,4,6-trione (210 mg, 1.34 mmol, 2.50 equiv), Pd(PPh₃)₄ (155 mg, 0.13 mmol, 0.25 equiv). The resulting solution was stirred for 2 h at 45° C. in an oil bath. The resulting mixture was concentrated under vacuum. The crude product (10 mL) was purified by Flash-Prep-HPLC. This resulted in 65 mg (23%) of Example 158 as a yellow solid.

Alternatively, Example 158 and its bis-tosylate salt (Compound 2 bis-tosylate)

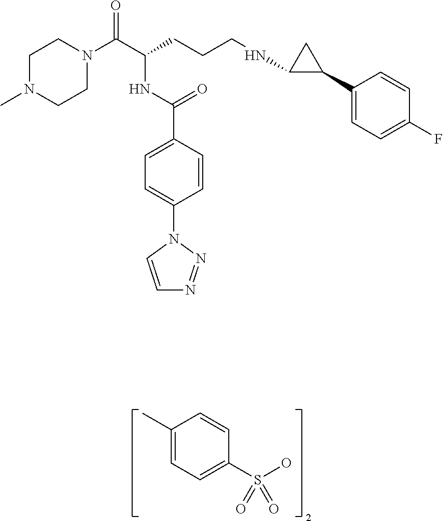

may be prepared by the method of Scheme III:

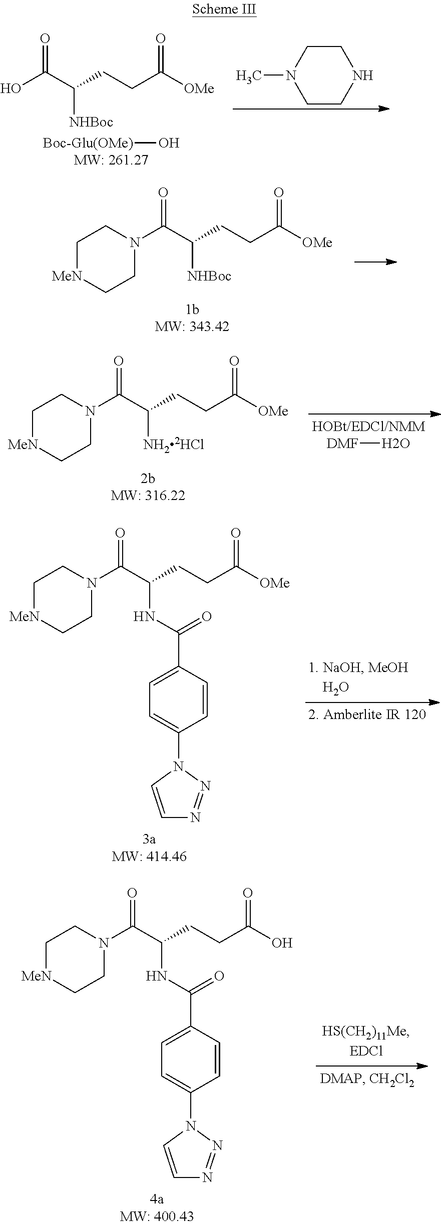

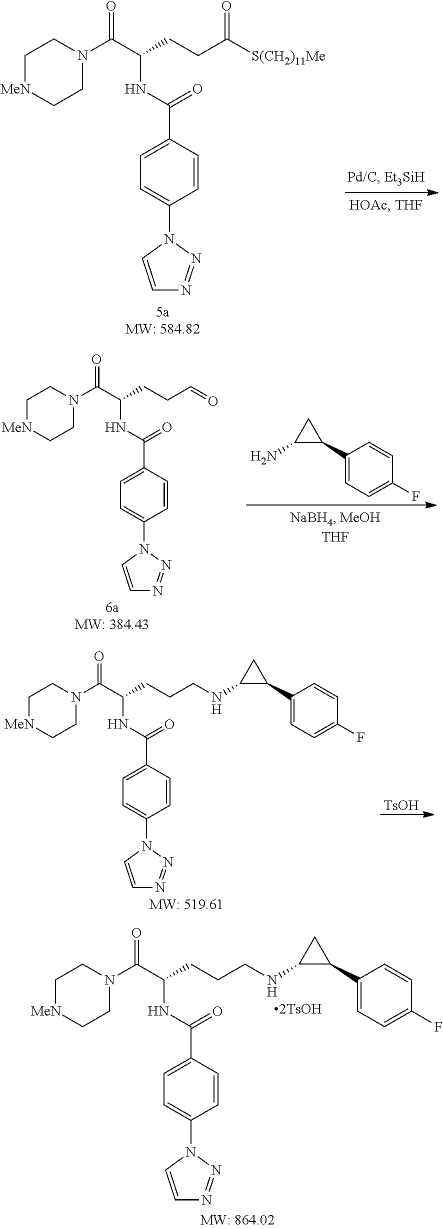

The following compounds may be synthesized using methods analogous to those described herein and known in the art, using appropriate starting materials and reagents. In the following structures, it should be understood that mixtures of or single isomers, such as racemic mixtures and alternate enantiomers, zwitterions, and the like may be prepared, e.g. by using appropriate L- or D-isomer, or chiral or achiral compound, as a staring material or reagent, or by employing a separation step.

Therefore, in certain embodiments in the compounds below, the configuration of the substituents off the cyclopropylamine is trans to the phenyl. In certain embodiments, the trans configuration is R, S; in others, it is S, R. Furthermore, in certain embodiments, the core contains a L-isomer, for example as shown in Formula II. Additional Examples include:

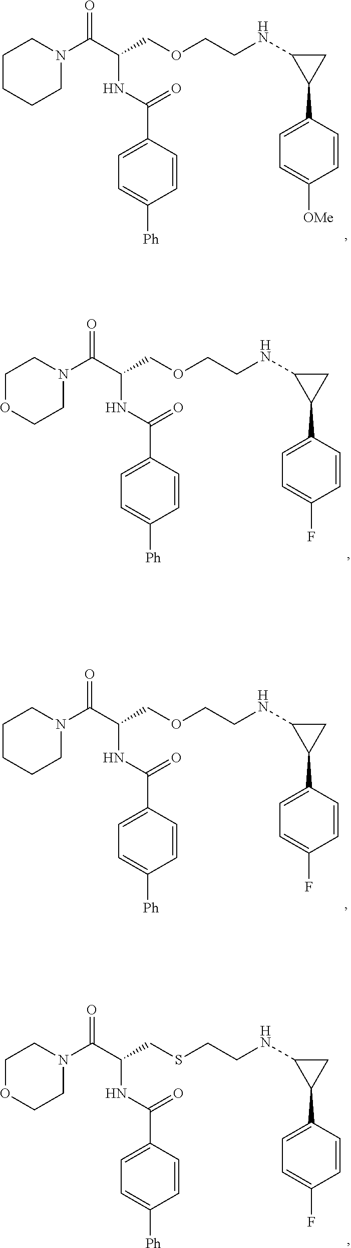

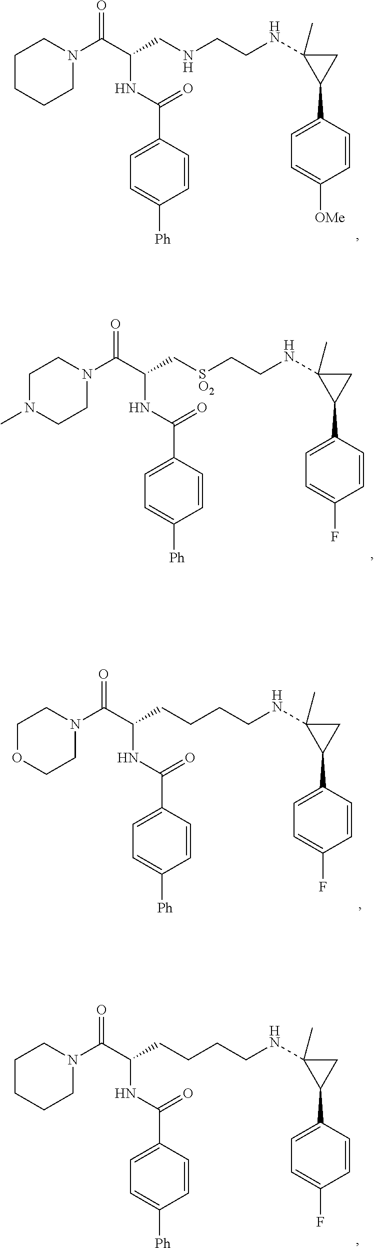

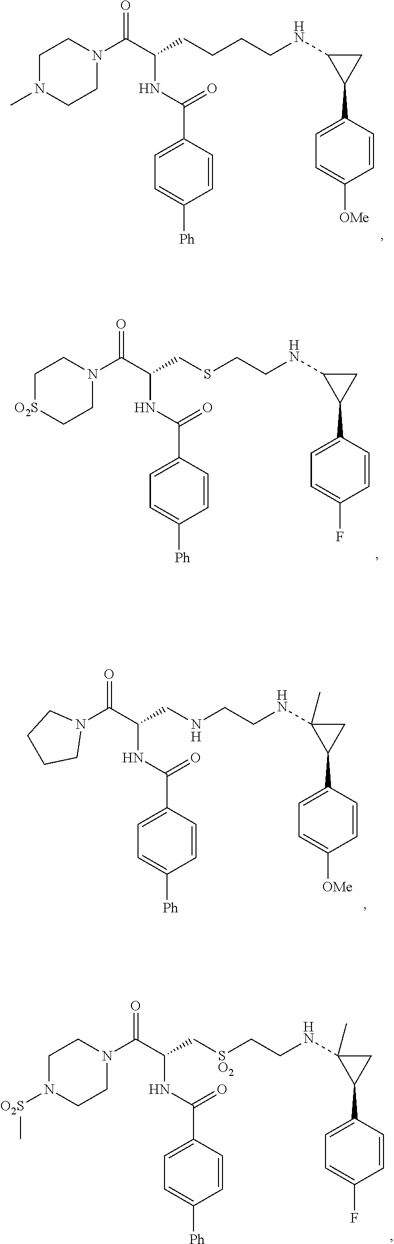

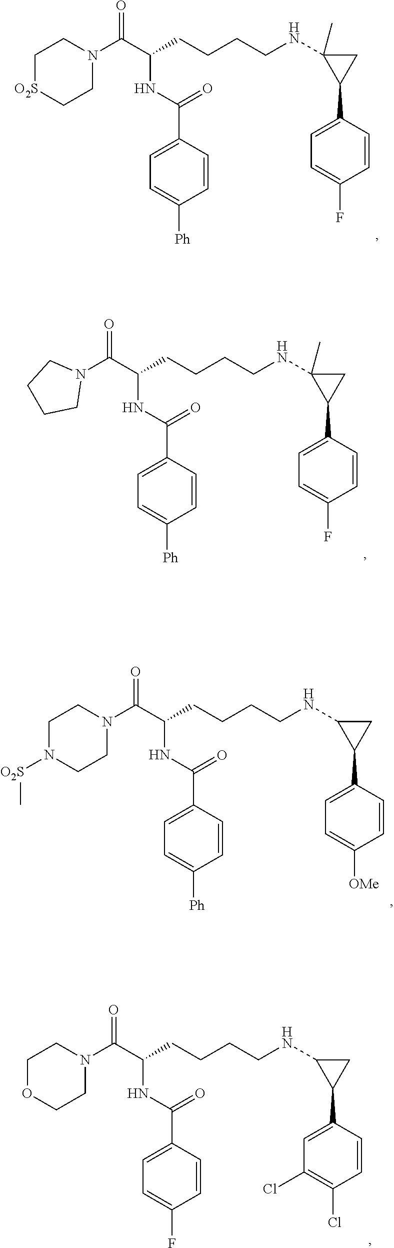

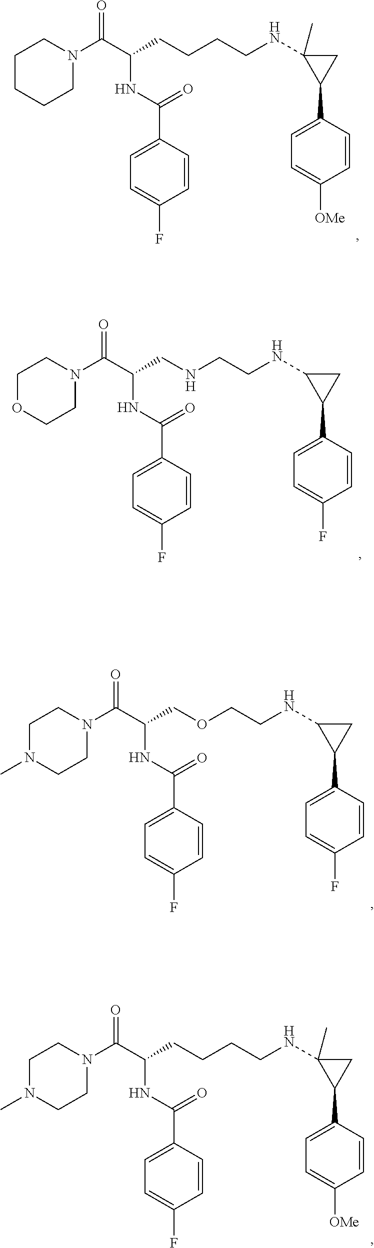

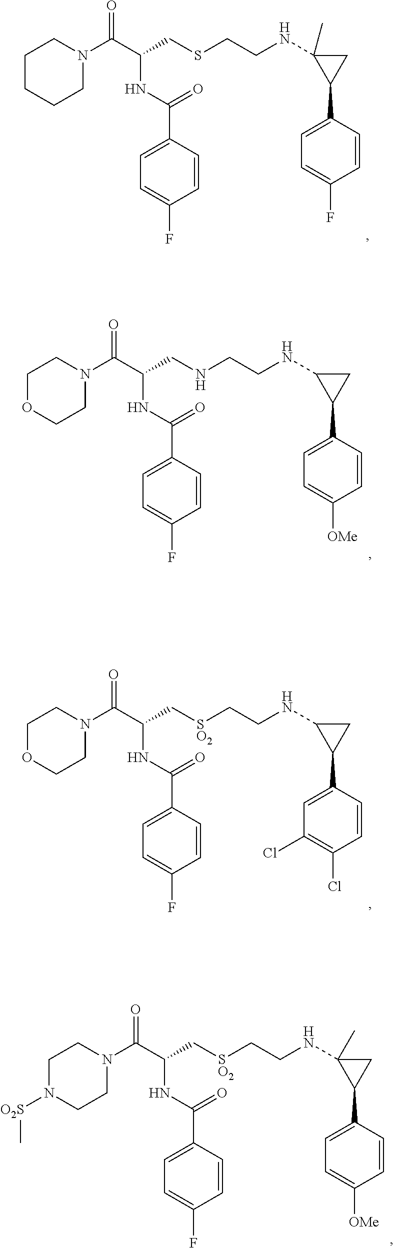

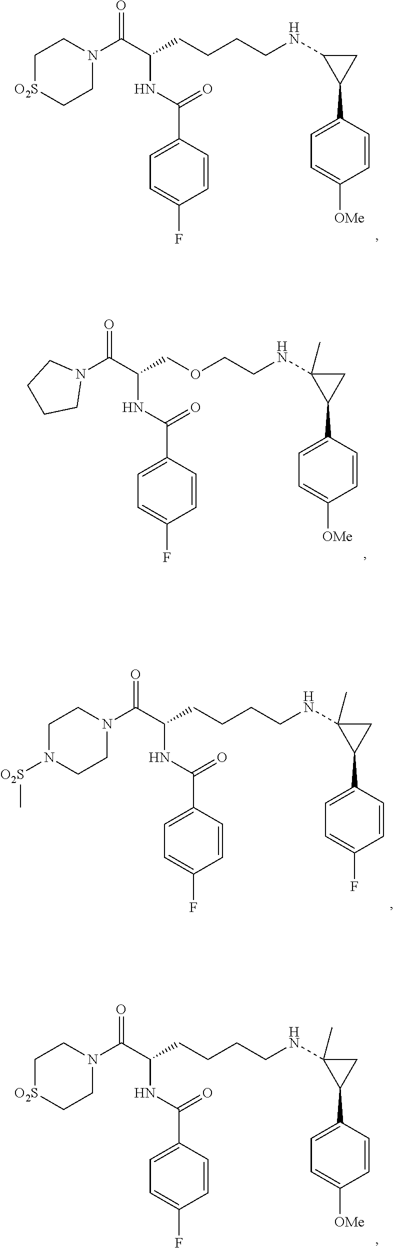

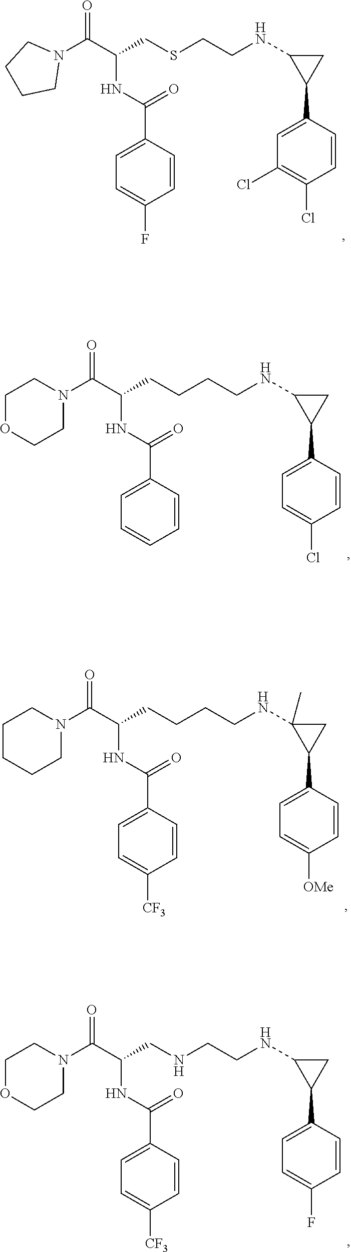

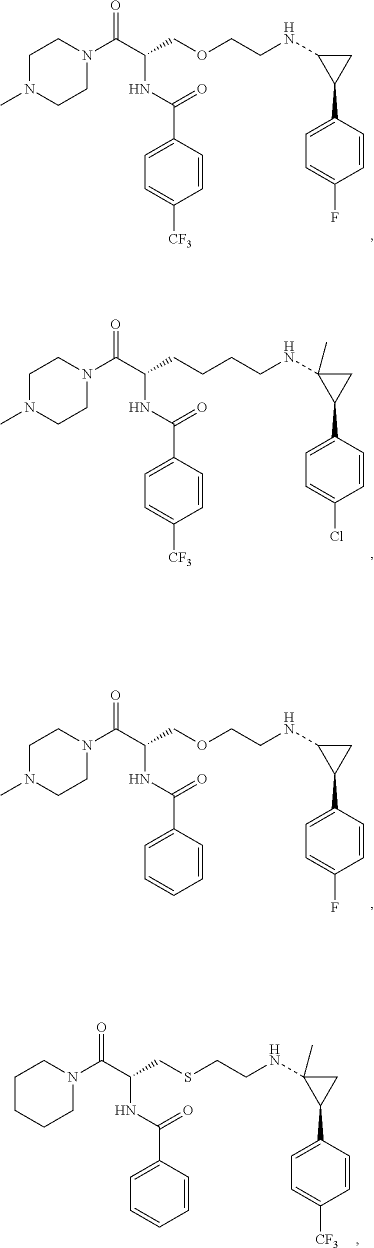

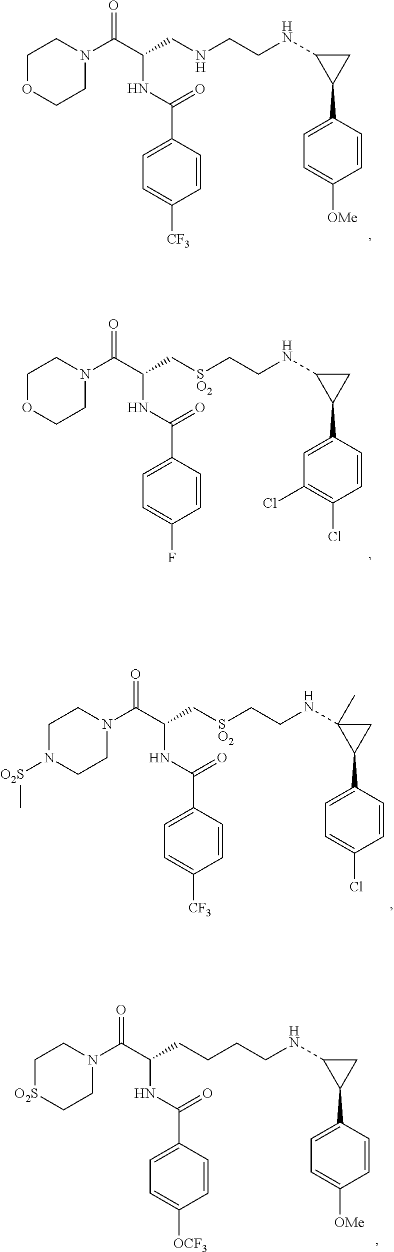

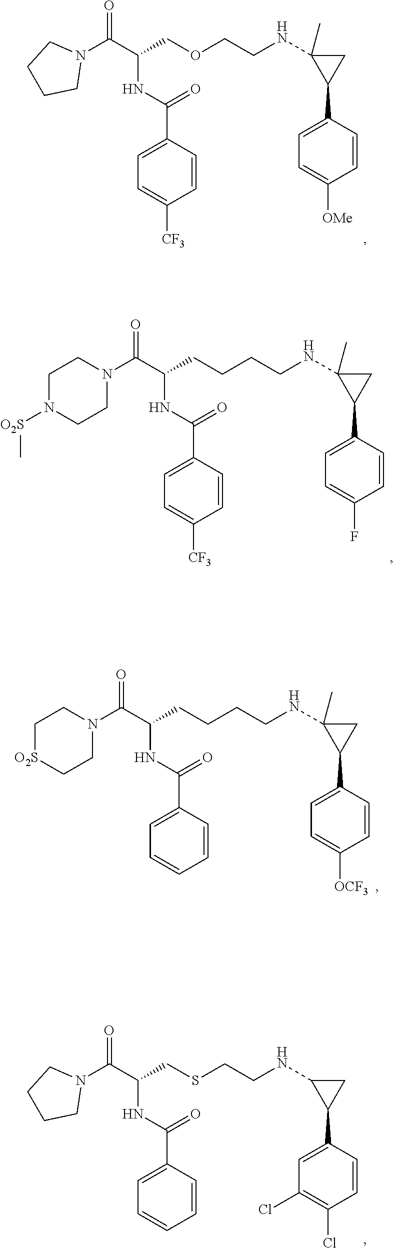

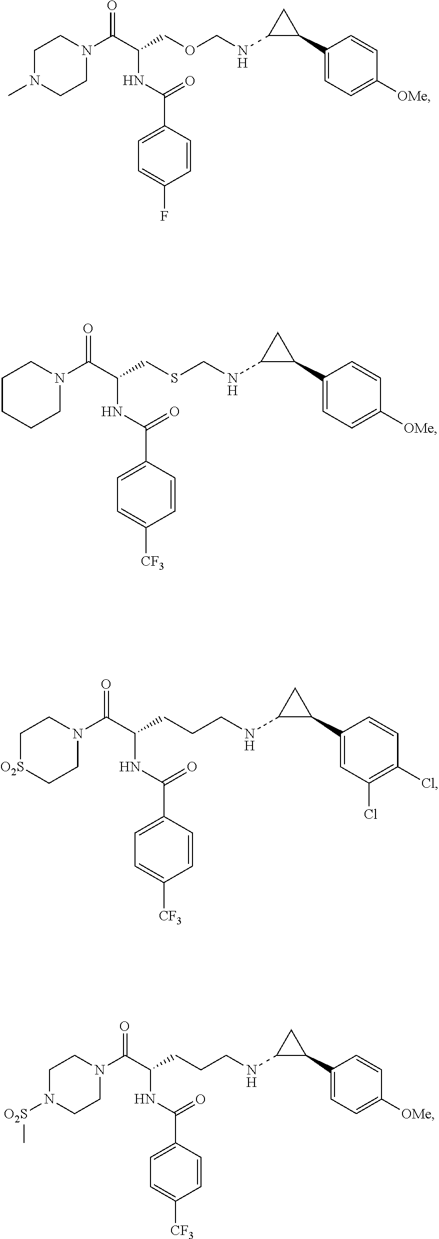

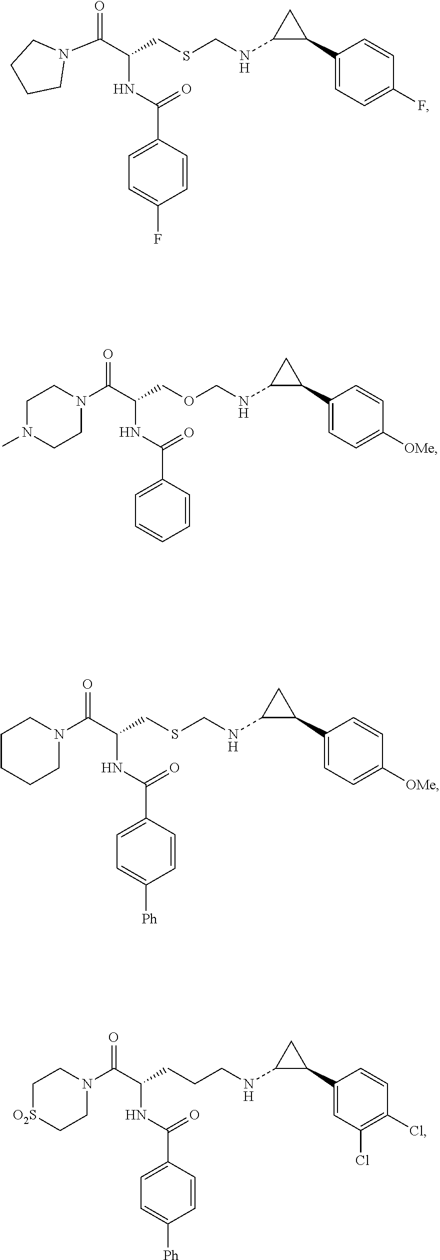

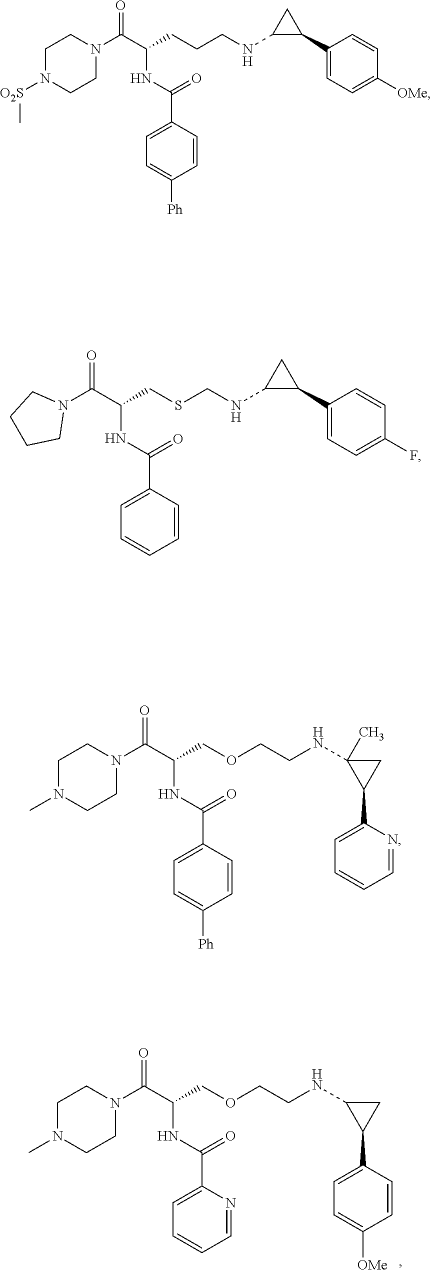

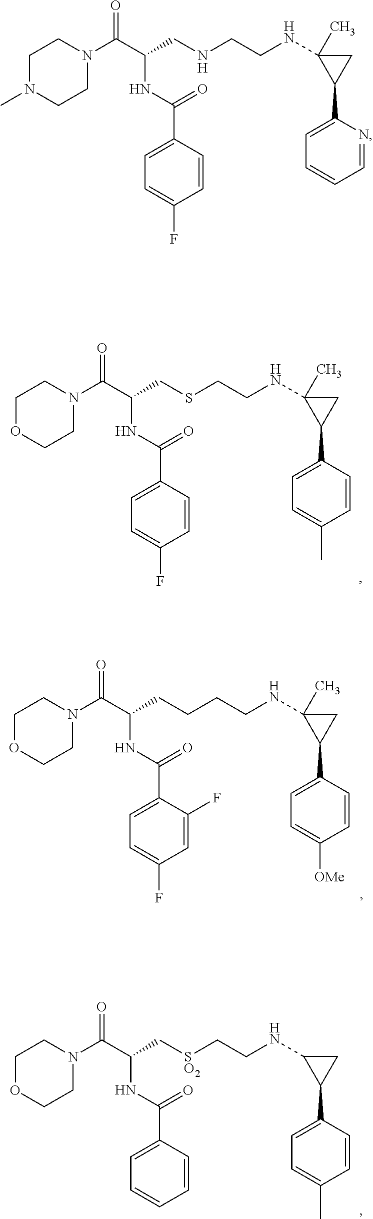

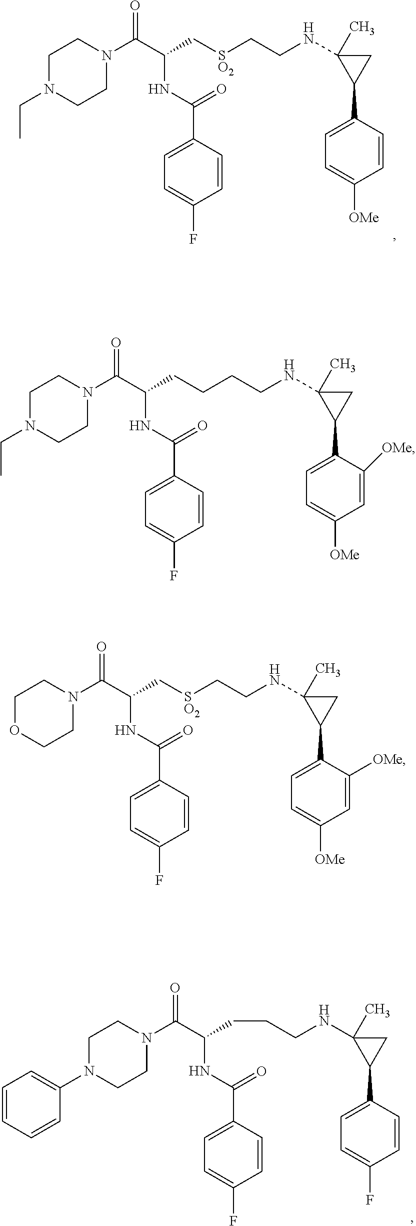

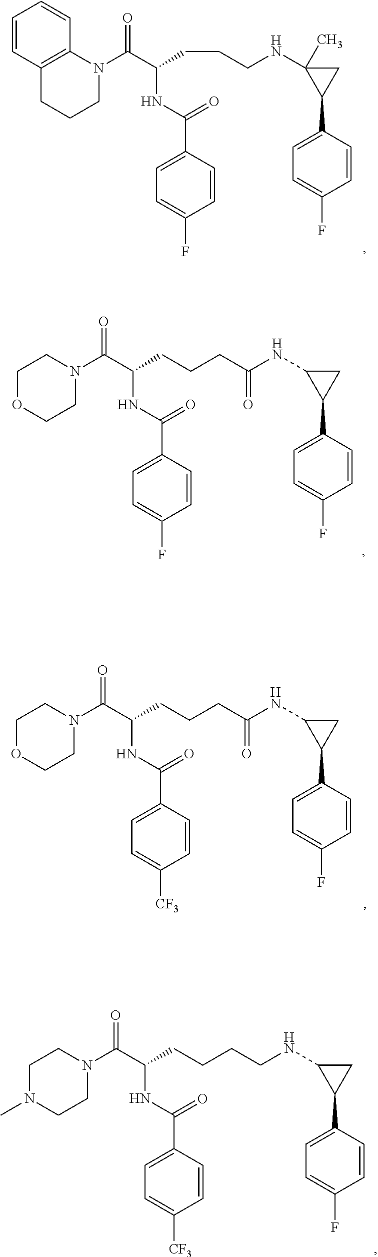

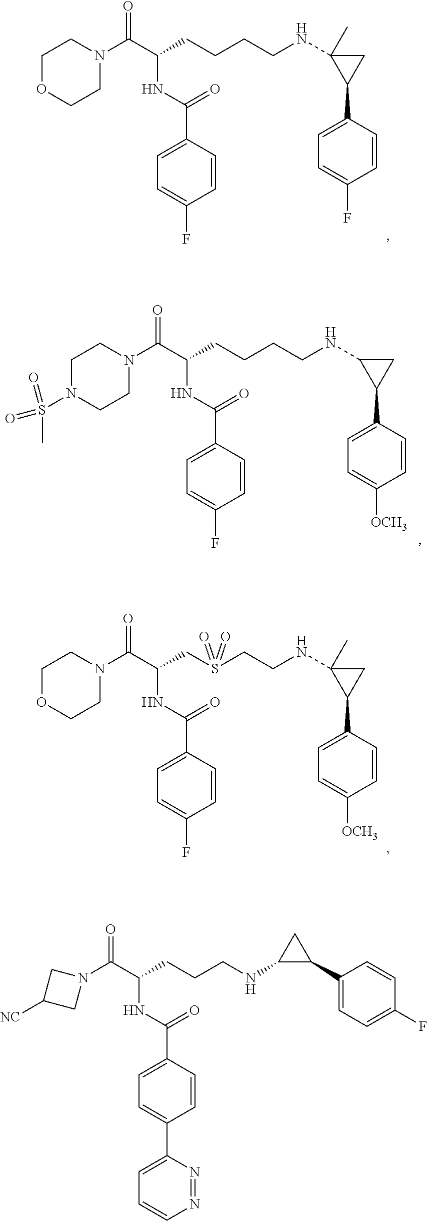

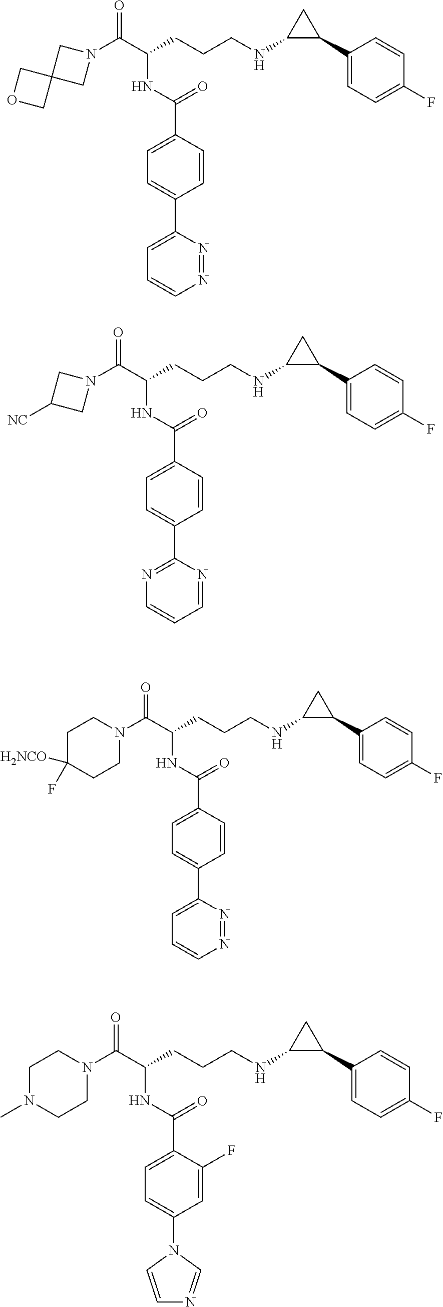

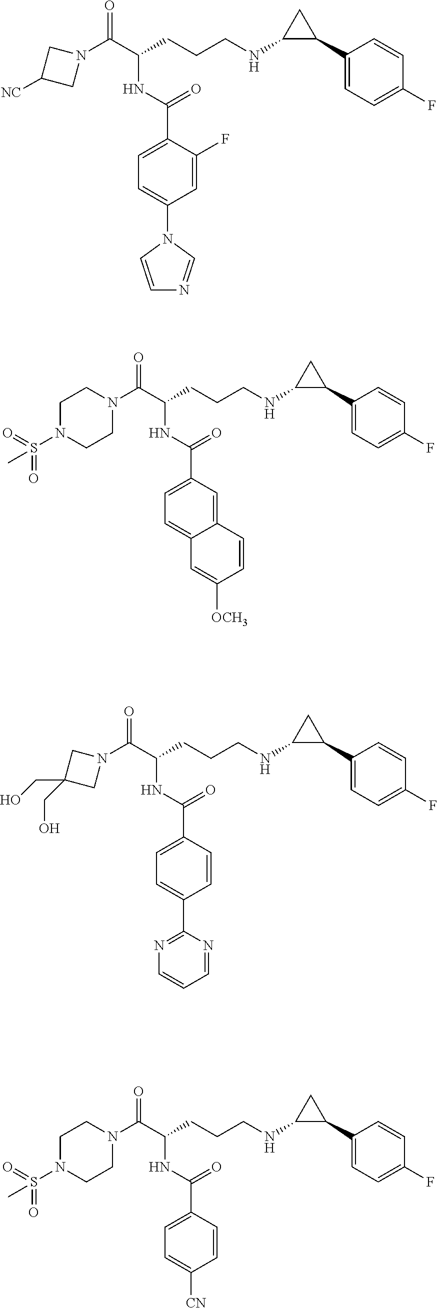 [SMILES: N#CC1CN(C(=O)[C@H](CCCN[C@@H]2C[C@H]2c2ccc(F)cc2)NC(=O)c2ccc(-n3ccnc3)cc2F)C1 ; COc1ccc2cc(C(=O)N[C@@H](CCCN[C@@H]3C[C@H]3c3ccc(F)cc3)C(=O)N3CCN(S(C)(=O)=O)CC3)ccc2c1 ; O=C(N[C@@H](CCCN[C@@H]1C[C@H]1c1ccc(F)cc1)C(=O)N1CC(CO)(CO)C1)c1ccc(-c2ncccn2)cc1 ; CS(=O)(=O)N1CCN(C(=O)[C@H](CCCN[C@@H]2C[C@H]2c2ccc(F)cc2)NC(=O)c2ccc(C#N)cc2)CC1]

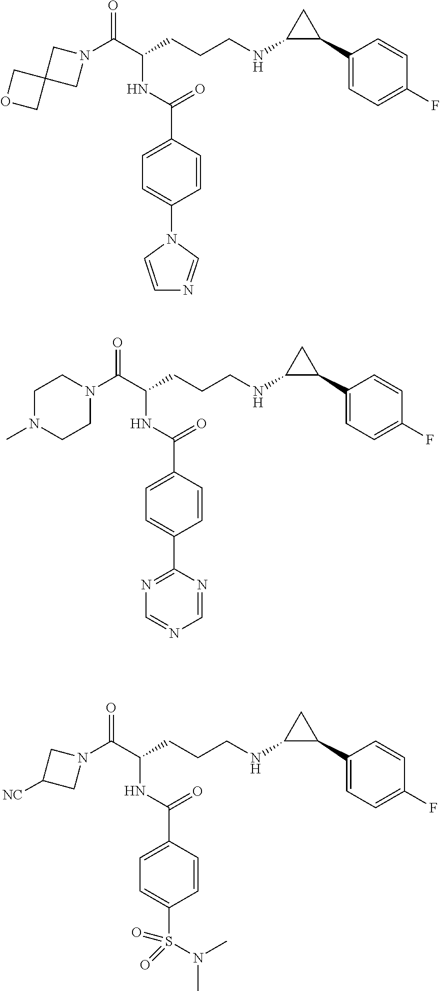 [SMILES: O=C(N[C@@H](CCCN[C@@H]1C[C@H]1c1ccc(F)cc1)C(=O)N1CC2(COC2)C1)c1ccc(-n2ccnc2)cc1 ; CN1CCN(C(=O)[C@H](CCCN[C@@H]2C[C@H]2c2ccc(F)cc2)NC(=O)c2ccc(-c3ncncn3)cc2)CC1 ; CN(C)S(=O)(=O)c1ccc(C(=O)N[C@@H](CCCN[C@@H]2C[C@H]2c2ccc(F)cc2)C(=O)N2CC(C#N)C2)cc1]

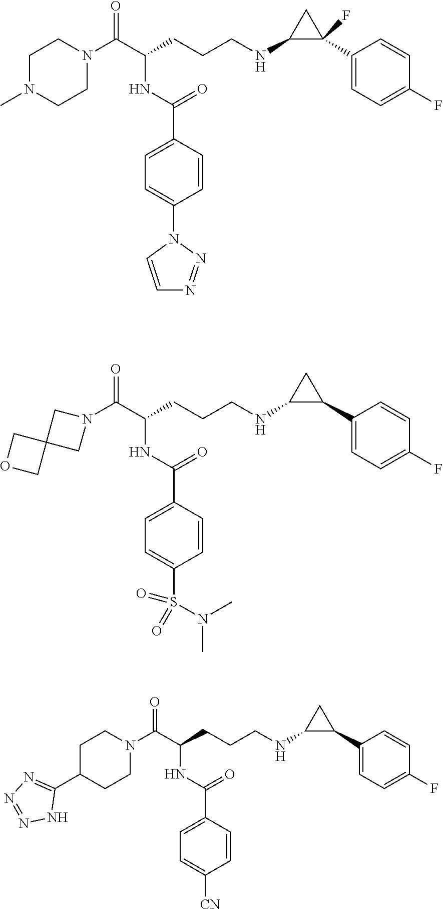 [SMILES: CN1CCN(C(=O)[C@H](CCCN[C@H]2C[C@]2(F)c2ccc(F)cc2)NC(=O)c2ccc(-n3ccnn3)cc2)CC1 ; CN(C)S(=O)(=O)c1ccc(C(=O)N[C@@H](CCCN[C@@H]2C[C@H]2c2ccc(F)cc2)C(=O)N2CC3(COC3)C2)cc1 ; N#Cc1ccc(C(=O)N[C@H](CCCN[C@@H]2C[C@H]2c2ccc(F)cc2)C(=O)N2CCC(c3nnn[nH]3)CC2)cc1]

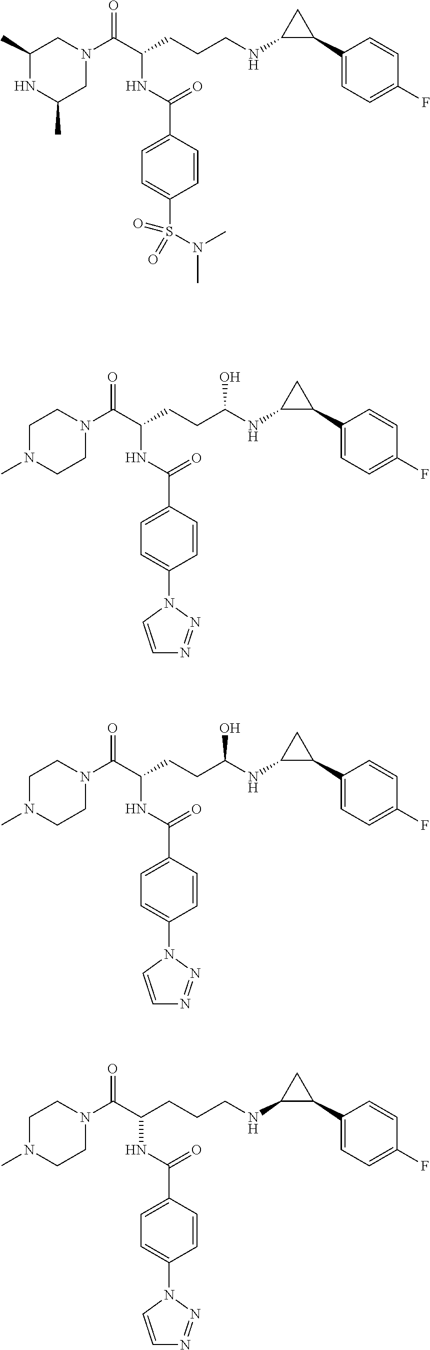 [SMILES: C[C@@H]1CN(C(=O)[C@H](CCCN[C@@H]2C[C@H]2c2ccc(F)cc2)NC(=O)c2ccc(S(=O)(=O)N(C)C)cc2)C[C@H](C)N1 ; CN1CCN(C(=O)[C@H](CC[C@H](O)N[C@@H]2C[C@H]2c2ccc(F)cc2)NC(=O)c2ccc(-n3ccnn3)cc2)CC1 ; CN1CCN(C(=O)[C@H](CC[C@@H](O)N[C@@H]2C[C@H]2c2ccc(F)cc2)NC(=O)c2ccc(-n3ccnn3)cc2)CC1 ; CN1CCN(C(=O)[C@H](CCCN[C@H]2C[C@H]2c2ccc(F)cc2)NC(=O)c2ccc(-n3ccnn3)cc2)CC1]

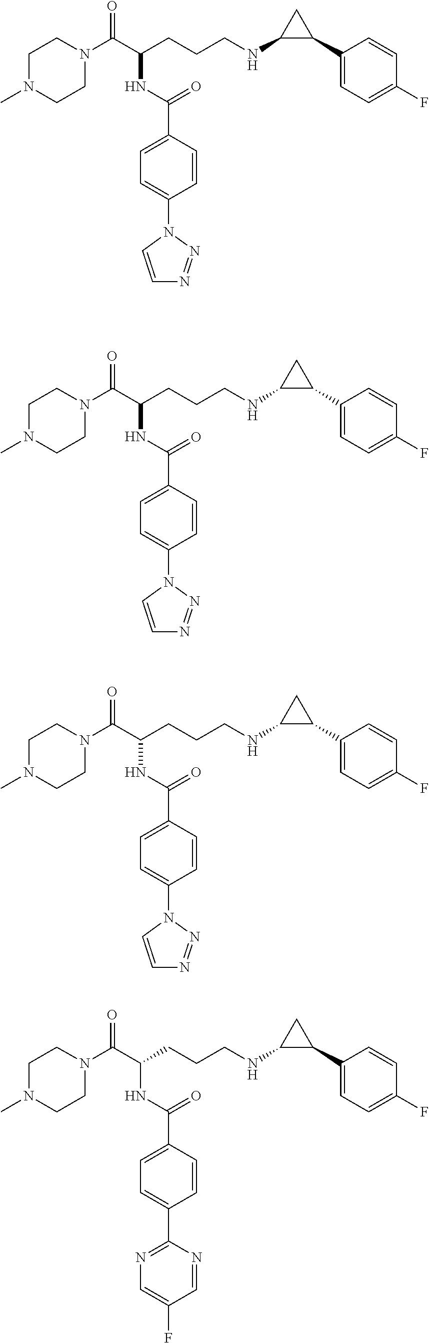 [SMILES: CN1CCN(C(=O)[C@@H](CCCN[C@H]2C[C@H]2c2ccc(F)cc2)NC(=O)c2ccc(-n3ccnn3)cc2)CC1 ; CN1CCN(C(=O)[C@@H](CCCN[C@@H]2C[C@@H]2c2ccc(F)cc2)NC(=O)c2ccc(-n3ccnn3)cc2)CC1 ; CN1CCN(C(=O)[C@H](CCCN[C@@H]2C[C@@H]2c2ccc(F)cc2)NC(=O)c2ccc(-n3ccnn3)cc2)CC1 ; CN1CCN(C(=O)[C@H](CCCN[C@@H]2C[C@H]2c2ccc(F)cc2)NC(=O)c2ccc(-c3ncc(F)cn3)cc2)CC1]

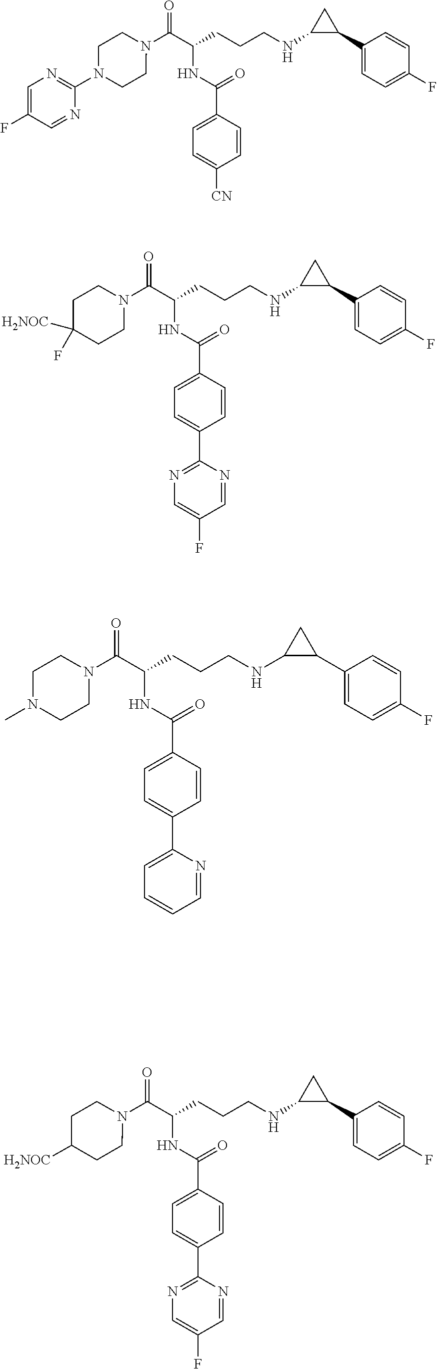 [SMILES: N#Cc1ccc(C(=O)N[C@@H](CCCN[C@@H]2C[C@H]2c2ccc(F)cc2)C(=O)N2CCN(c3ncc(F)cn3)CC2)cc1 ; NC(=O)C1(F)CCN(C(=O)[C@H](CCCN[C@@H]2C[C@H]2c2ccc(F)cc2)NC(=O)c2ccc(-c3ncc(F)cn3)cc2)CC1 ; CN1CCN(C(=O)[C@H](CCCNC2CC2c2ccc(F)cc2)NC(=O)c2ccc(-c3ccccn3)cc2)CC1 ; NC(=O)C1CCN(C(=O)[C@H](CCCN[C@@H]2C[C@H]2c2ccc(F)cc2)NC(=O)c2ccc(-c3ncc(F)cn3)cc2)CC1]

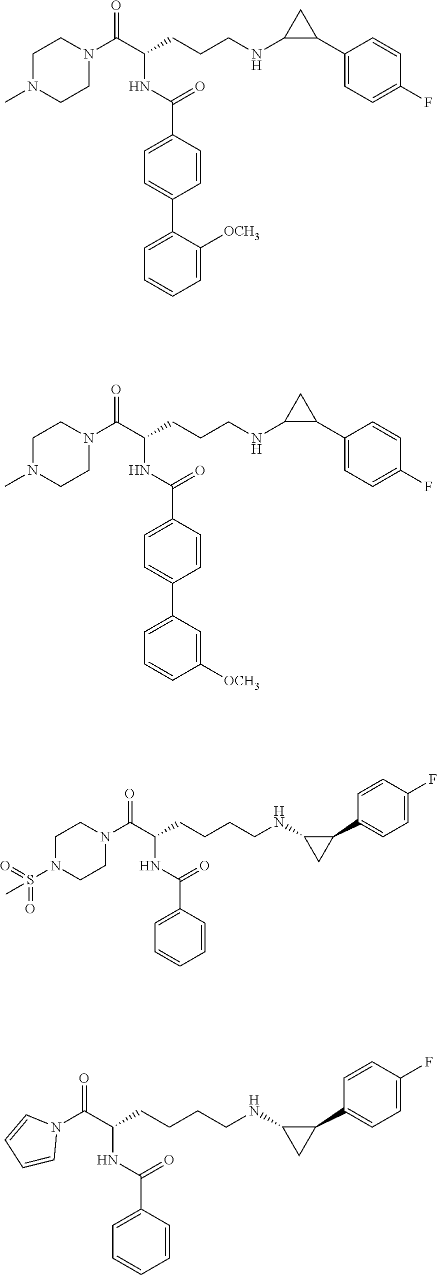 [SMILES: COc1ccccc1-c1ccc(C(=O)N[C@@H](CCCNC2CC2c2ccc(F)cc2)C(=O)N2CCN(C)CC2)cc1 ; COc1cccc(-c2ccc(C(=O)N[C@@H](CCCNC3CC3c3ccc(F)cc3)C(=O)N3CCN(C)CC3)cc2)c1 ; CS(=O)(=O)N1CCN(C(=O)[C@H](CCCCN[C@H]2C[C@@H]2c2ccc(F)cc2)NC(=O)c2ccccc2)CC1 ; O=C(N[C@@H](CCCCN[C@H]1C[C@@H]1c1ccc(F)cc1)C(=O)n1cccc1)c1ccccc1]

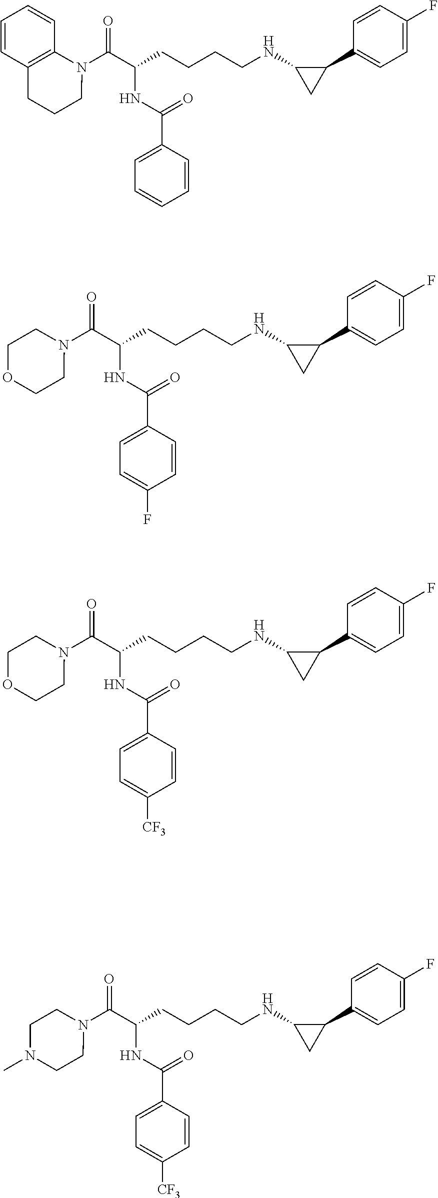 [SMILES: O=C(N[C@@H](CCCCN[C@H]1C[C@@H]1c1ccc(F)cc1)C(=O)N1CCCc2ccccc21)c1ccccc1 ; O=C(N[C@@H](CCCCN[C@H]1C[C@@H]1c1ccc(F)cc1)C(=O)N1CCOCC1)c1ccc(F)cc1 ; O=C(N[C@@H](CCCCN[C@H]1C[C@@H]1c1ccc(F)cc1)C(=O)N1CCOCC1)c1ccc(C(F)(F)F)cc1 ; CN1CCN(C(=O)[C@H](CCCCN[C@H]2C[C@@H]2c2ccc(F)cc2)NC(=O)c2ccc(C(F)(F)F)cc2)CC1]

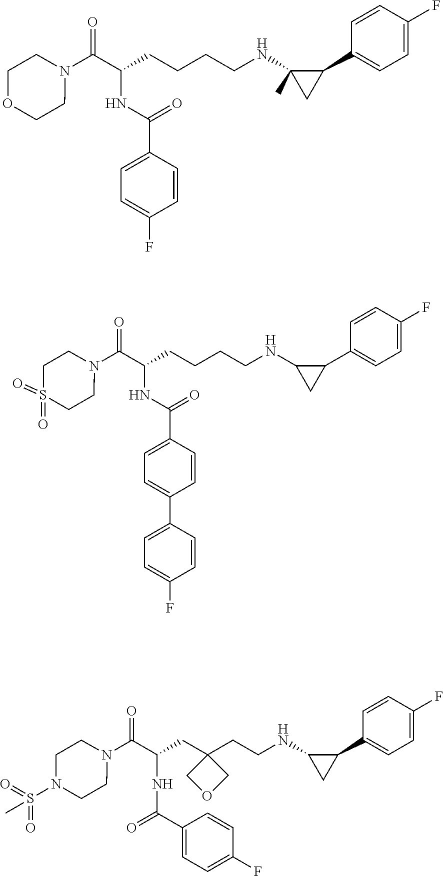 [SMILES: C[C@]1(NCCCC[C@H](NC(=O)c2ccc(F)cc2)C(=O)N2CCOCC2)C[C@@H]1c1ccc(F)cc1 ; O=C(N[C@@H](CCCCNC1CC1c1ccc(F)cc1)C(=O)N1CCS(=O)(=O)CC1)c1ccc(-c2ccc(F)cc2)cc1 ; CS(=O)(=O)N1CCN(C(=O)[C@H](CC2(CCN[C@H]3C[C@@H]3c3ccc(F)cc3)COC2)NC(=O)c2ccc(F)cc2)CC1]

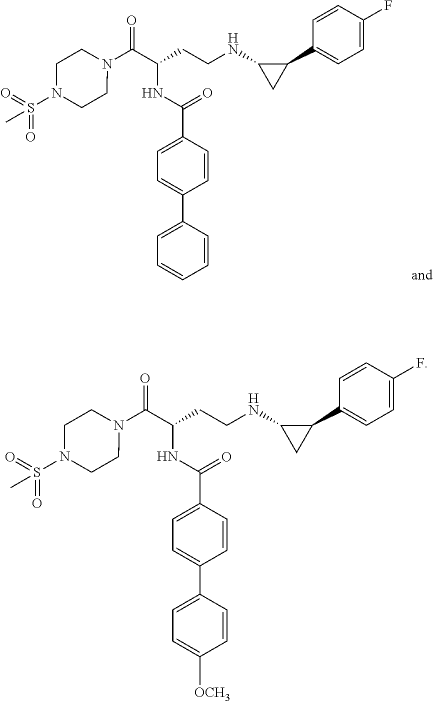 [SMILES: CS(=O)(=O)N1CCN(C(=O)[C@H](CCN[C@H]2C[C@@H]2c2ccc(F)cc2)NC(=O)c2ccc(-c3ccccc3)cc2)CC1 ; COc1ccc(-c2ccc(C(=O)N[C@@H](CCN[C@H]3C[C@@H]3c3ccc(F)cc3)C(=O)N3CCN(S(C)(=O)=O)CC3)cc2)cc1]

Certain embodiments of the invention use compounds of the formula (I):

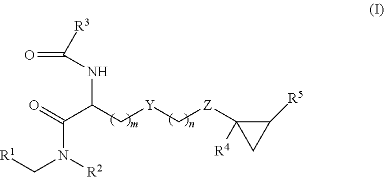

or a salt thereof, wherein:

Y is chosen from a bond, NR^{4a}, O, C(O)NH, NHC(O), S, SO₂, and CH₂:

Z is chosen from a bond, NR^{4b}, O, C(O)NH, NHC(O), S, SO₂, and CH₂;

m is an integer from 0 to 5;

n is an integer from 0 to 3:

R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkvlalkyl and R¹ and R², together with the nitrogen to which they attach, form a nitrogen-containing heterocycloalkyl or heteroaryl ring, which may be optionally substituted with between 0 and 3 R⁶ groups;

R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups:

R⁴, R^{4a}, and R^{4b} are independently chosen from hydrogen, alkyl, alkenyl, alkynyl, and cycloalkyl:

R⁵ is chosen from aryl and heteroaryl, any of which may be optionally substituted with between 0 and 3 R⁶ groups:

each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and

R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl:

with the proviso that when Y═CH₂ and Z═R^{4b}, then m+n≠3.

In certain embodiments, the compound has Formula IIa or IIb:

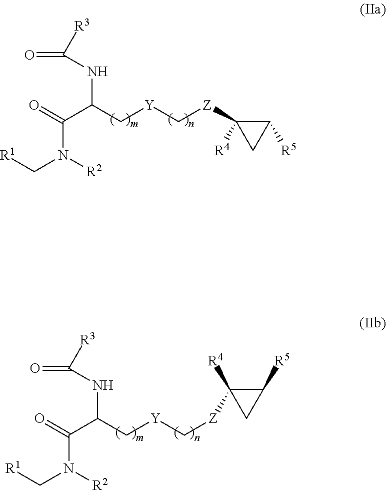

or a salt thereof, wherein:

Y is chosen from a bond, NR^{4a}, O, C(O)NH, NHC(O), S, SO₂, and CH₂;

Z is chosen from a bond, NR^{4b}, O, C(O)NH, NHC(O), S, SO₂, and CH₂;

m is an integer from 0 to 5;

n is an integer from 0 to 3;

R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups; R; is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;

R⁴, R^{4a}, and R^{4b} are independently chosen from hydrogen, alkyl, alkenyl, alkynyl, and cycloalkyl:

R⁵ is chosen from aryl and heteroaryl, any of which may be optionally substituted with between 0 and 3 R⁶ groups;

each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino. COR⁷, SO₂R⁷, NHSO₂R⁷, NHSO₂NHR⁷, NHCOR⁷, NHCONHR⁷, CONHR⁷, and CONR⁷R⁸; and

R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;

with the proviso that when Y═CH₂ and Z═R^{4b}, then m+n≠3.

In certain embodiments, the compound has Formula IIIa or IIIb:

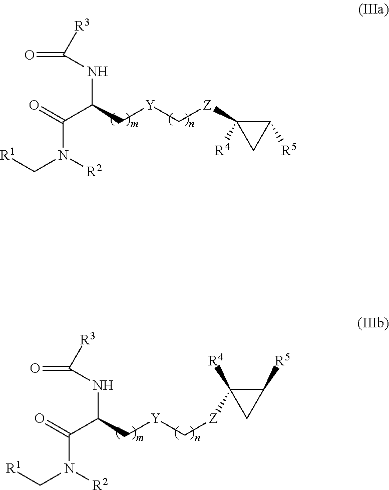

or a salt thereof, wherein:

Y is chosen from a bond, NR^{4a}, O, C(O)NH, NHC(O), S, SO₂, and CH₂;

Z is chosen from a bond, NR^{4b}, O, C(O)NH, NHC(O), S, SO₂, and CH₂;

m is an integer from 0 to 5;

n is an integer from 0 to 3;

R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;

R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;

R⁴, R^{4a}, and R^{4b} are independently chosen from hydrogen, alkyl, alkenyl, alkynyl, and cycloalkyl;

R⁵ is chosen from aryl and heteroaryl, any of which may be optionally substituted with between 0 and 3 R⁶ groups;

each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and

R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;

with the proviso that when Y═CH₂ and Z═R^{4b}, then m+n≠3.

In certain embodiments. Z is NR^{4b}.

In certain embodiments, R^{4b} is chosen from methyl and hydrogen.

In certain embodiments, the alkyl, whether by itself or as a named part of another non-cyclic substituent, is C₁-C₈ alkyl.

In certain embodiments, R³ is chosen from aryl, arylalkyl, heteroaryl, and heteroarylalkyl, any of which may be optionally substituted with between 0 and 3 R⁶ groups.

In certain embodiments, R³ is chosen from aryl and heteroaryl, any of which may be optionally substituted with between 0 and 3 R⁶ groups.

In certain embodiments, m is an integer from 0 to 1; Y is chosen from NR^{4a}, O, S, SO₂, and CH₂; n is an integer from 1 to 3; and R^{4a} is chosen from hydrogen and alkyl.

In certain embodiments, m is 0; Y is CH₂; and n is an integer from 1 to 2.

In certain embodiments, n is 1. In certain embodiments, n is 2. In certain embodiments, n is 3.

In certain embodiments, R³ is 5-6 membered monocyclic or 8-12 membered bicyclic heteroaryl, in which between one and five ring members may be heteroatoms chosen from N, O, and S, and which may be optionally substituted with between 0 and 3 R⁶ groups.

In certain embodiments, R³ is 5-6 membered monocyclic heteroaryl, in which between one and four ring members may be heteroatoms chosen from N, O, and S, and which may be optionally substituted with between 0 and 3 R⁶ groups.

In certain embodiments, each R⁶ is chosen from lower alkyl, halogen, lower alkoxy, OCF₃ and CF₃.

In certain embodiments, R³ is chosen from

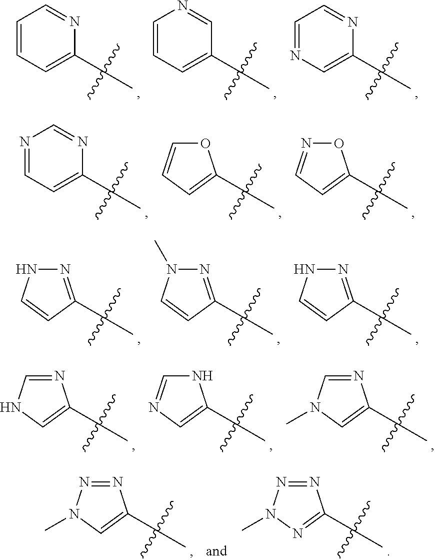

In certain embodiments, R⁴ is hydrogen.

In certain embodiments, R⁴ is methyl.

In certain embodiments, the nitrogen-containing heterocycloalkyl or heteroaryl ring formed by R¹ and R² together with the nitrogen to which they are attached contains 3 to eight atoms.

In certain embodiments, R¹ and R² are taken together to form a nitrogen-containing heterocycloalkyl, which may be optionally substituted with between 0 and 3 R⁶ groups.

In certain embodiments, the nitrogen-containing heterocycloalkyl formed by R¹ and R² together with the nitrogen to which they are attached is chosen from:

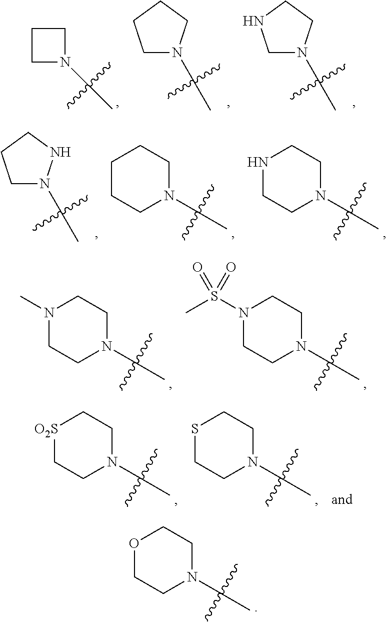

In certain embodiments, the nitrogen-containing heterocycloalkyl formed by R¹ and R² together with the nitrogen to which they are attached is chosen from:

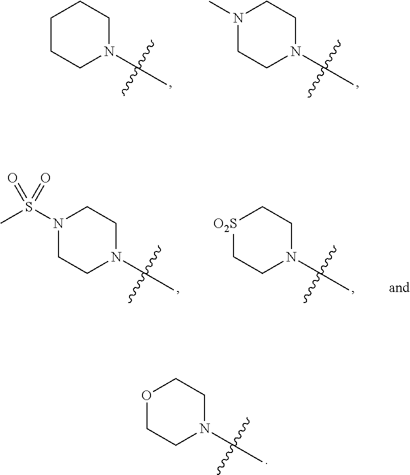

In certain embodiments, the nitrogen-containing heterocycloalkyl formed by R¹ and R² together with the nitrogen to which they are attached is

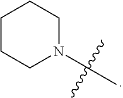 [SMILES: *N1CCCCC1]

In certain embodiments, the nitrogen-containing heterocycloalkyl formed by R¹ and R² together with the nitrogen to which they are attached is

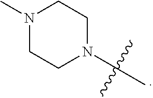 [SMILES: *N1CCN(C)CC1 ; "."]

In certain embodiments, the nitrogen-containing heterocycloalkyl formed by R¹ and R² together with the nitrogen to which they are attached is

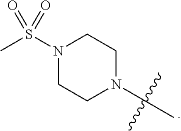 [SMILES: *N1CCN(S(C)(=O)=O)CC1 ; "."]

In certain embodiments, the nitrogen-containing heterocycloalkyl formed by R¹ and R² together with the nitrogen to which they are attached is

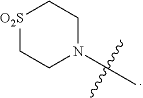 [SMILES: *N1CCS(=O)(=O)CC1 ; "."]

In certain embodiments, the nitrogen-containing heterocycloalkyl formed by R¹ and R² together with the nitrogen to which they are attached is

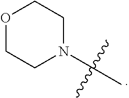 [SMILES: *N1CCOCC1 ; "."]

In certain embodiments, n is 2 or 3.

In certain embodiments. R¹ and R² are taken together with the nitrogen to which they are attached form a nitrogen-containing heteroaryl, which may be optionally substituted with between 0 and 3 R⁶ groups.

In certain embodiments, the nitrogen-containing heteroaryl is chosen from pyrrole, imidazole, and pyrazole.

In certain embodiments, R⁵ is aryl, which may be optionally substituted with between 0 and 3 R⁶ groups.

In certain embodiments, R⁵ is phenyl, which may be optionally substituted with between 0 and 3 R⁶ groups.

In certain embodiments, n is 2 or 3.

In certain embodiments, R⁵ is heteroaryl, which may be optionally substituted with between 0 and 3 R⁶ groups.

In certain embodiments, R⁵ is a 5-6 membered monocyclic or 8-12 membered bicyclic heteroaryl, in which between one and five ring members may be heteroatoms chosen from N, O, and S, and which may be optionally substituted with between 0 and 3 R⁶ groups.

In certain embodiments. R⁵ is a 5-6 membered monocyclic heteroaryl, in which between one and five ring members may be heteroatoms chosen from N, O, and S, and which may be optionally substituted with 1 or 2 R⁶ groups.

In certain embodiments, R⁵ is chosen from:

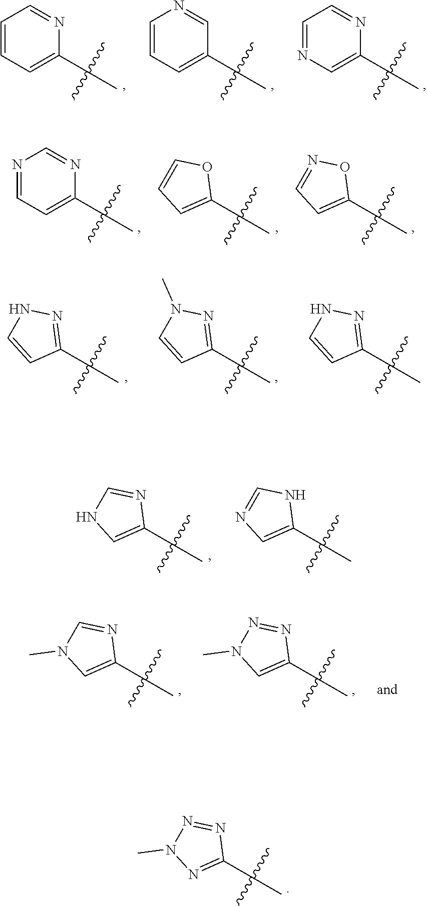

In certain embodiments, n is 2 or 3.

In certain embodiments. R³ is aryl, optionally substituted with between 0 and 3 R⁶ groups.

In certain embodiments, R³ is chosen from phenyl and biphenyl, either of which may be optionally substituted with between 0 and 3 R⁶ groups.

In certain embodiments,

m is an integer from 0 to 1;

Y is chosen from NR^{4a}, O, S, SO₂, and CH₂:

n is an integer from 1 to 3; and

R^{4a} is chosen from hydrogen and alkyl.

In certain embodiments,

m is 0;

Y is CH₂; and

n is an integer from 1 to 3.

In certain embodiments, n is 1. In certain embodiments, n is 2. In certain embodiments, n is 3.

In certain embodiments, R⁶ is chosen from lower alkyl, halogen, lower alkoxy, OCF₃ and CF₃.

In certain embodiments, R⁴ is hydrogen.

In certain embodiments, R⁴ is methyl.

In certain embodiments, n is 2 or 3.

In certain embodiments, the nitrogen-containing heterocycloalkyl or heteroaryl ring formed by R¹ and R² together with the nitrogen to which they are attached contains 3 to eight atoms.

In certain embodiments, R¹ and R² are taken together to form a nitrogen-containing heterocycloalkyl, which may be optionally substituted with between 0 and 3 R⁶ groups.

In certain embodiments, the nitrogen-containing heterocycloalkyl formed by R¹ and R² together with the nitrogen to which they are attached is chosen from:

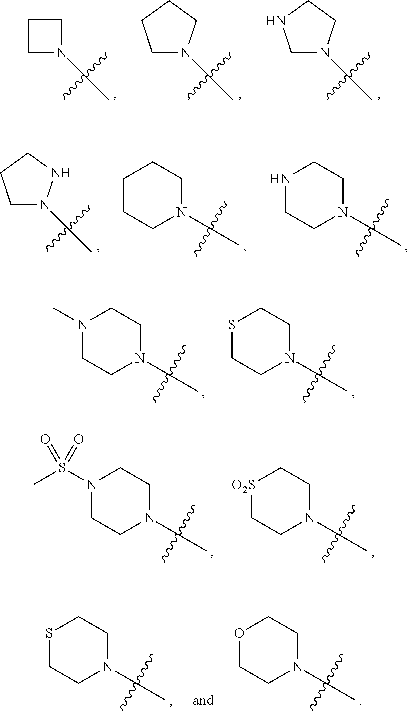

In certain embodiments, the nitrogen-containing heterocycloalkyl formed by R¹ and R² together with the nitrogen to which they are attached is chosen from:

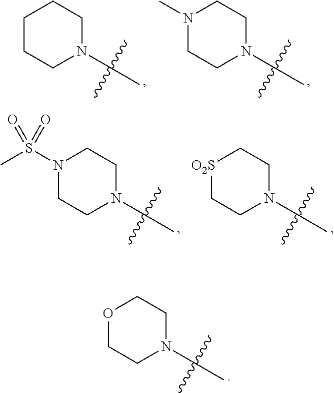

In certain embodiments, the nitrogen-containing heterocycloalkyl formed by R¹ and R² together with the nitrogen to which they are attached is

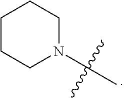 [SMILES: *N1CCCCC1 ; "."]

In certain embodiments, the nitrogen-containing heterocycloalkyl formed by R¹ and R² together with the nitrogen to which they are attached is

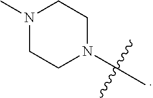 [SMILES: *N1CCN(C)CC1 ; "."]

In certain embodiments, the nitrogen-containing heterocycloalkyl formed by R¹ and R² together with the nitrogen to which they are attached is

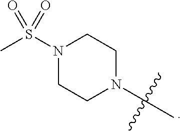 [SMILES: *N1CCN(S(C)(=O)=O)CC1 ; "."]

In certain embodiments, the nitrogen-containing heterocycloalkyl formed by R¹ and R² together with the nitrogen to which they are attached is

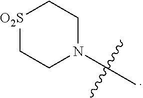 [SMILES: *N1CCS(=O)(=O)CC1 ; "."]

In certain embodiments, the nitrogen-containing heterocycloalkyl formed by R¹ and R² together with the nitrogen to which they are attached is

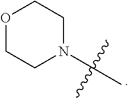 [SMILES: *N1CCOCC1 ; "."]

In certain embodiments, n is 2 or 3.

In certain embodiments, R¹ and R² taken together form a nitrogen-containing heteroaryl which may be optionally substituted with between 0 and 3 R⁶ groups.

In certain embodiments, the nitrogen-containing heteroaryl is chosen from pyrrole, imidazole, and pyrazole.

In certain embodiments, R⁵ is aryl, which may be optionally substituted with between 0 and 3 R⁶ groups, each of which is independently chosen from lower alkyl, halogen, lower alkoxy, OCF₃ and CF₃.

In certain embodiments, R⁵ is phenyl, which may be optionally substituted with between 0 and 3 R⁶ groups, each of which is independently chosen from lower alkyl, halogen, lower alkoxy, OCF₃ and CF₃.

In certain embodiments, n is 2 or 3.

In certain embodiments. R⁵ is heteroaryl, which may be optionally substituted with between 0 and 3 R⁶ groups.

In certain embodiments, R⁵ is a 5-6 membered monocyclic or 8-12 membered bicyclic heteroaryl, in which between one and five ring members may be heteroatoms chosen from N, O, and S. and which may be optionally substituted with between 0 and 3 R⁶ groups, each of which is independently chosen from lower alkyl, halogen, lower alkoxy, OCF₃ and CF₃.

In certain embodiments, R⁵ is a 5-6 membered monocyclic heteroaryl, in which between one and five ring members may be heteroatoms chosen from N, O, and S, and which may be optionally substituted with 1 or 2 R⁶ groups, each of which is independently, if present, a lower alkyl groups.

In certain embodiments, R⁵ is chosen from:

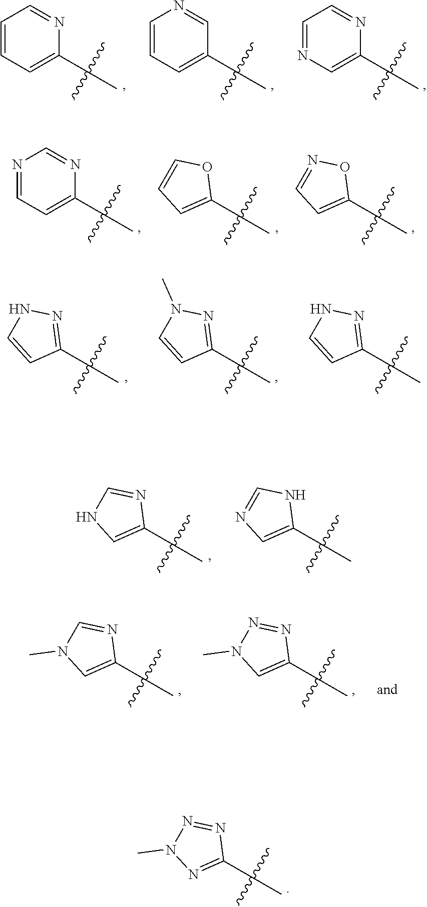

In certain embodiments, wherein n is 2 or 3.

Certain embodiments of the invention use compounds of the formula (IV):

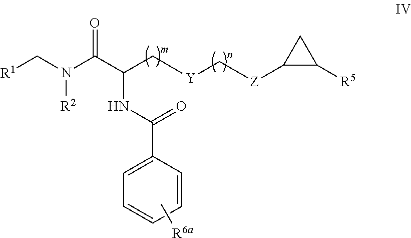

or a salt, polymorph, or solvate thereof, wherein:

Y is chosen from a bond, NR^{4a}, O, C(O)NH, NHC(O), S, SO₂, CHOH, and CH₂;

Z is chosen from a bond, NR^{4b}, O, C(O)NH, NHC(O), S, SO₂, and CH₂;

m is chosen from 0, 1, 2, 3, 4, and 5;

n is chosen from 0, 1, 2, and 3;

R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;

R^{4a} and R^{4b} are independently chosen from hydrogen, alkyl, alkenyl, alkynyl, and cycloalkyl;

R⁵ is chosen from aryl and heteroaryl, any of which may be optionally substituted with between 0 and 3 R⁶ groups;

R^{6a} is chosen from heteroaryl, cyano, and S(O)₂N(CH₃)₂;

each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and

R⁷ and R⁸ are independently chosen from hydrogen, aryl, and lower alkyl or R⁷ and R⁸ may be taken together to form a nitrogen-containing heterocycloalkyl or heteroaryl ring, which may be optionally substituted with lower alkyl;

with the proviso that when Y═CH₂ and Z═R^{4b}, then m+n≠3.

In certain embodiments, Z in Formula IV is NR^{4b}.

In certain embodiments of Formula IV, R^{4b} is chosen from methyl and hydrogen.

In certain embodiments of Formula IV, R^{4b} is hydrogen.

In certain embodiments of Formula IV, the alkyl, whether by itself or as a named part of another non-cyclic substituent, is C₁-C₈ alkyl.

In certain embodiments of Formula IV, m is 0; Y is CH₂; and n is chosen from 0, 1, and 2. In certain embodiments, n is 2.

In certain embodiments of Formula IV, R¹ and R² are each independently chosen from alkyl, aminoalkyl, alkylsulfonylalkyl, alkoxyalkyl, and heteroaryl, and R¹ and R², together with the nitrogen to which they attach, form a nitrogen-containing heterocycloalkyl or heteroaryl ring, which may be optionally substituted with between 0 and 3 R⁶ groups.

In certain embodiments of Formula IV, the nitrogen-containing heterocycloalkyl or heteroaryl ring formed by R¹ and R² together with the nitrogen to which they are attached contains 3 to eight atoms.

In certain embodiments of Formula IV, R¹ and R² are taken together to form a nitrogen-containing heterocycloalkyl, which may be optionally substituted with between 0 and 3 R⁶ groups.

In certain embodiments of Formula IV, the nitrogen-containing heterocycloalkyl is optionally substituted with between 0 and 3 R⁶ groups chosen from alkyl, halogen, CONH₂, SO₂CH³, cyano, spiro-heterocycloalkyl, and oxo.

In certain embodiments of Formula IV, the nitrogen-containing heterocycloalkyl is chosen from:

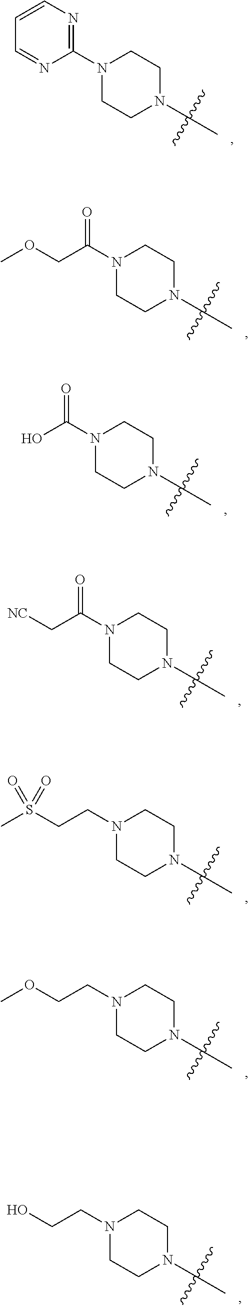

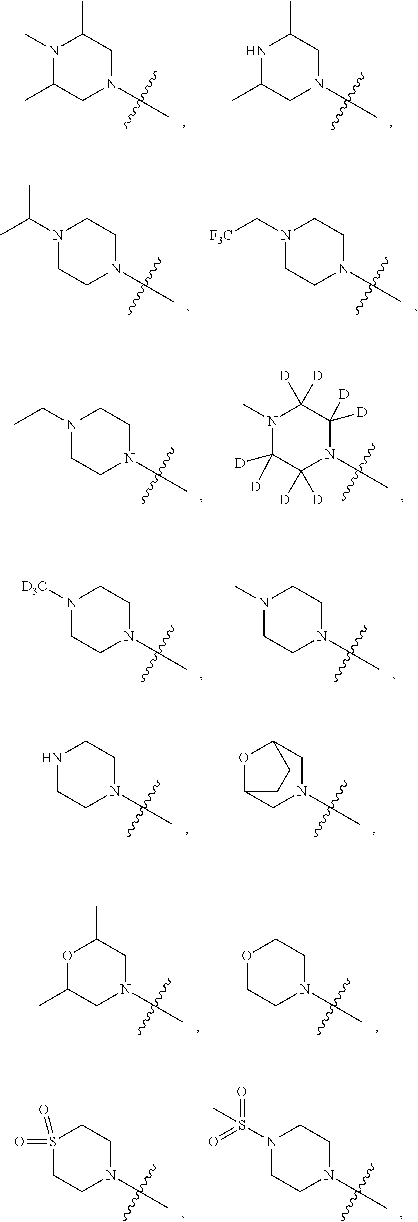

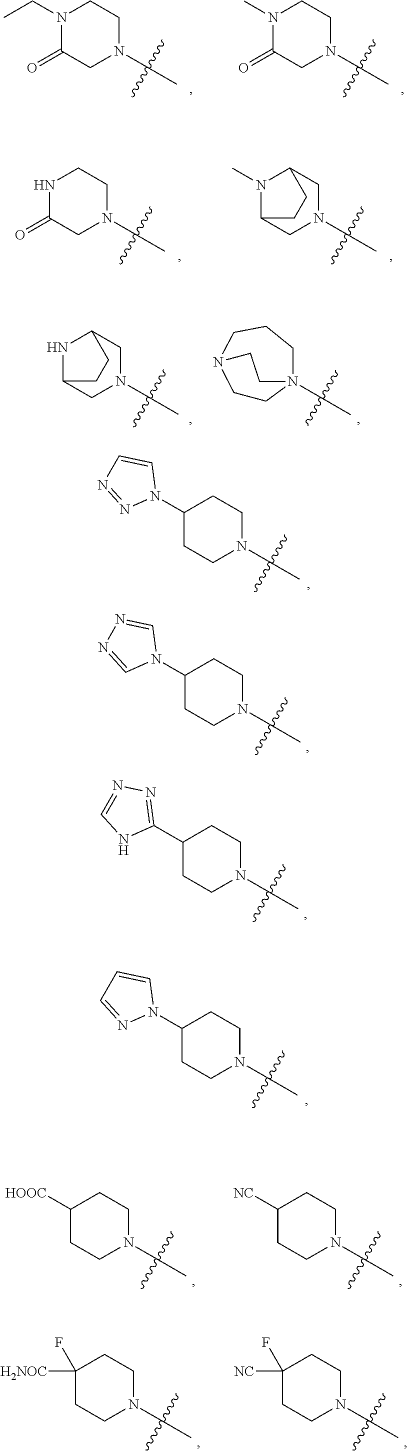

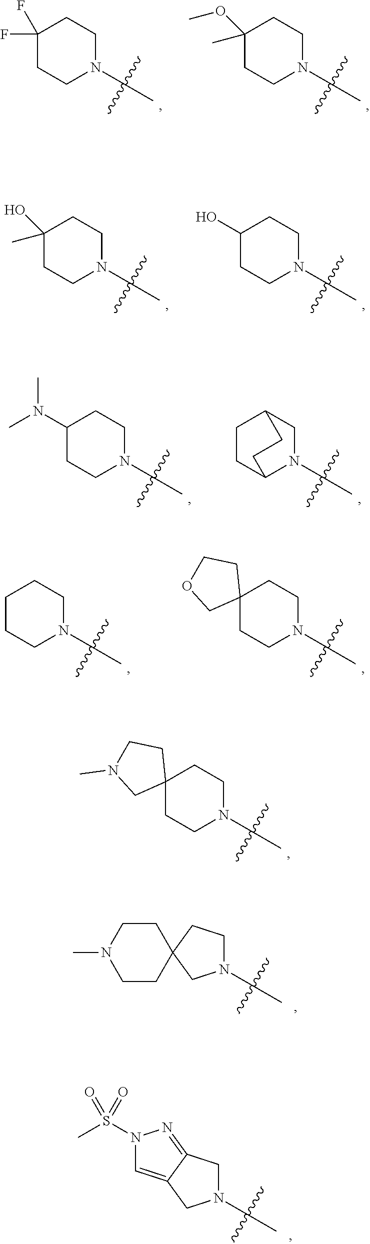

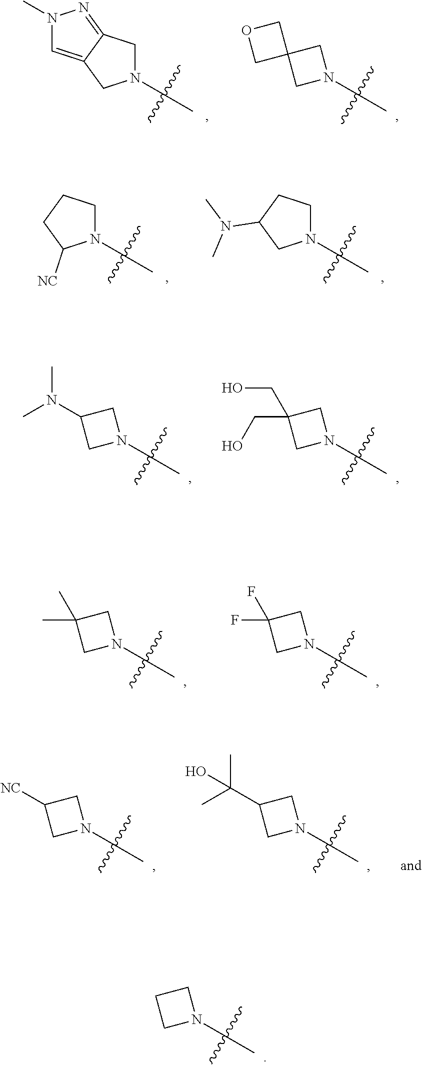

In certain embodiments of Formula IV, the nitrogen-containing heterocycloalkyl is chosen from:

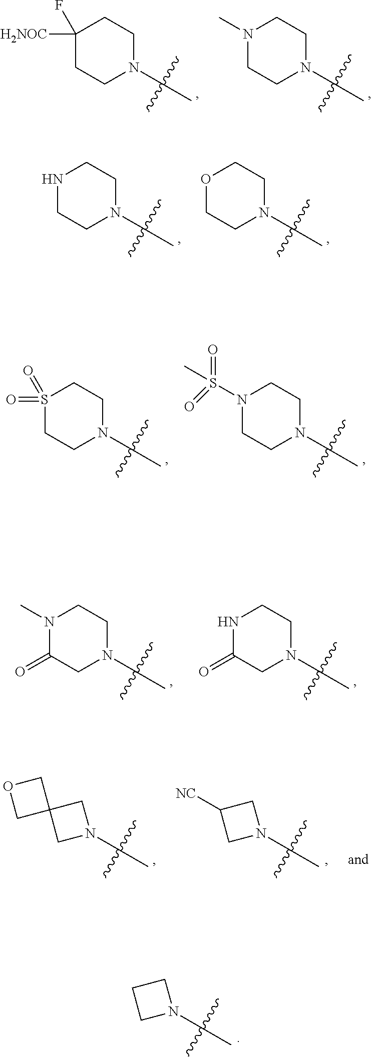

In certain embodiments of Formula IV, the nitrogen-containing heterocycloalkyl is:

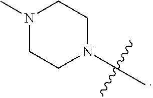 [SMILES: *N1CCN(C)CC1 ; "."]

In certain embodiments of Formula IV, each R^{6a} is chosen from cyano, S(O)₂N(CH₃)₂,

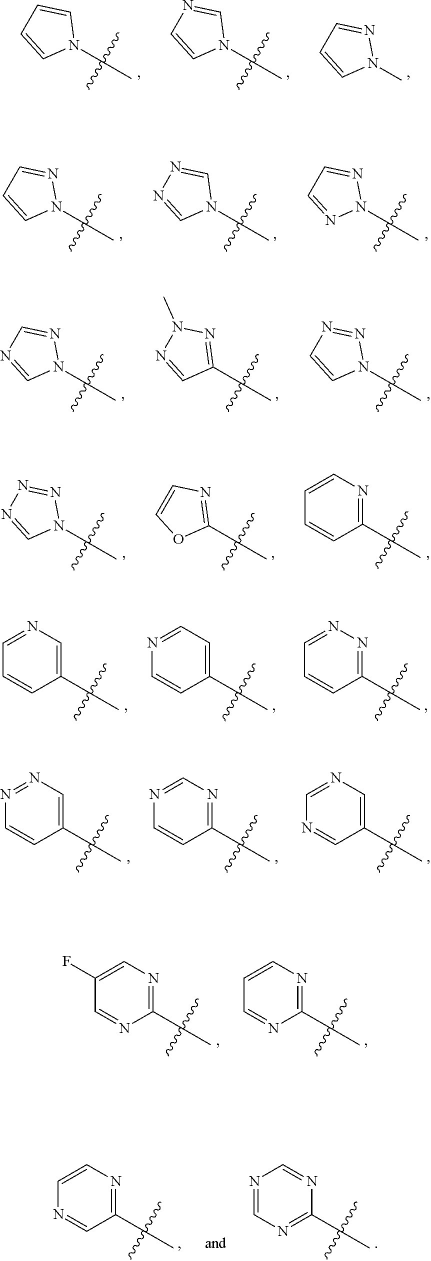

In certain embodiments of Formula IV, R⁵ is phenyl, which may be optionally substituted with between 0 and 3 R⁶ groups.

In certain embodiments of Formula IV, wherein R⁵ is:

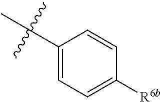

wherein R^{6b} is chosen from halogen, hydroxy, and methoxy.

In certain embodiments of Formula IV, R^{6b} is chosen from fluoro, methoxy, and hydroxy.

In certain embodiments of Formula IV, R^{6b} is fluoro.

In certain embodiments of Formula IV, the compound of Formula IV is a compound of Formula V:

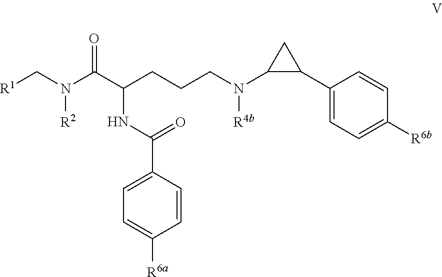

or a salt, polymorph, or solvate thereof, wherein:

R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;

R^{4a} is chosen from hydrogen, alkyl, alkenyl, alkynyl, and cycloalkyl;

R^{6a} is chosen from heteroaryl, cyano, and S(O)₂N(CH₃)₂;

each R⁶ and R^{6b} is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and

R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl.

In certain embodiments of Formula V, R^{4b} is chosen from methyl and hydrogen.

In certain embodiments of Formula V, R^{4b} is hydrogen.

In certain embodiments of Formula V, each R^{6a} is chosen from cyano,

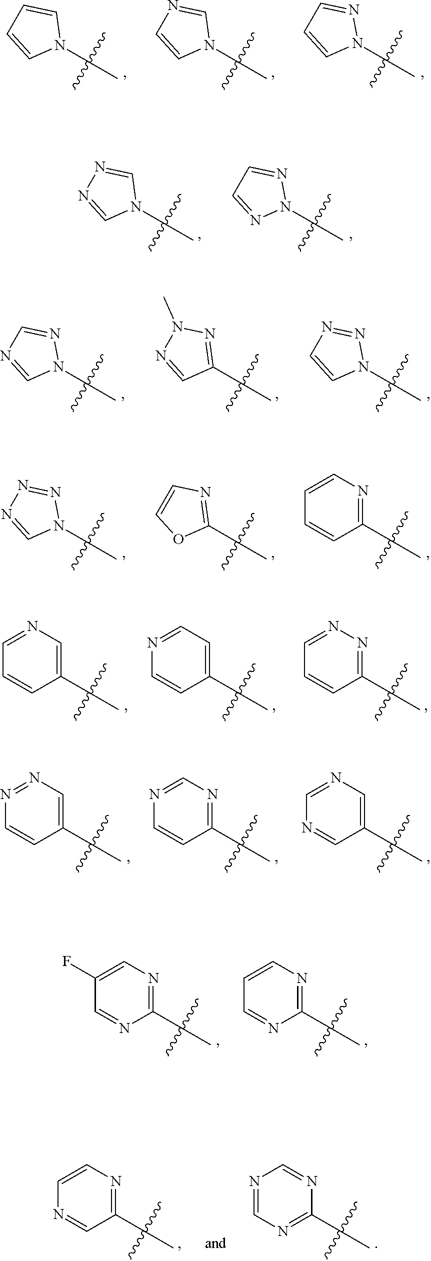

In certain embodiments of Formula V, R¹ and R² are each independently chosen from alkyl, aminoalkyl, alkylsulfonylalkyl, alkoxyalkyl, and heteroaryl, and R¹ and R², together with the nitrogen to which they attach, form a nitrogen-containing heterocycloalkyl or heteroaryl ring, which may be optionally substituted with between 0 and 3 R⁶ groups.

In certain embodiments of Formula V, the nitrogen-containing heterocycloalkyl or heteroaryl ring formed by R¹ and R² together with the nitrogen to which they are attached contains 3 to eight atoms.

In certain embodiments of Formula V, R¹ and R² are taken together to form a nitrogen-containing heterocycloalkyl, which may be optionally substituted with between 0 and 3 R⁶ groups.

In certain embodiments of Formula V, the nitrogen-containing heterocycloalkyl is chosen from:

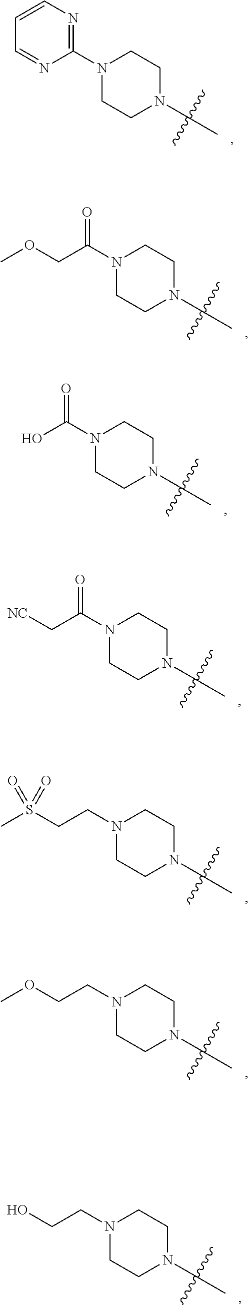

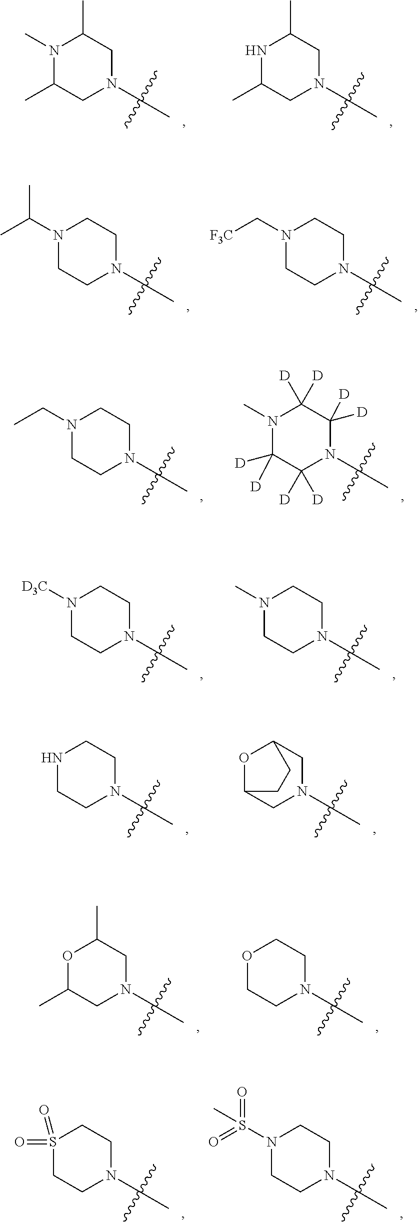

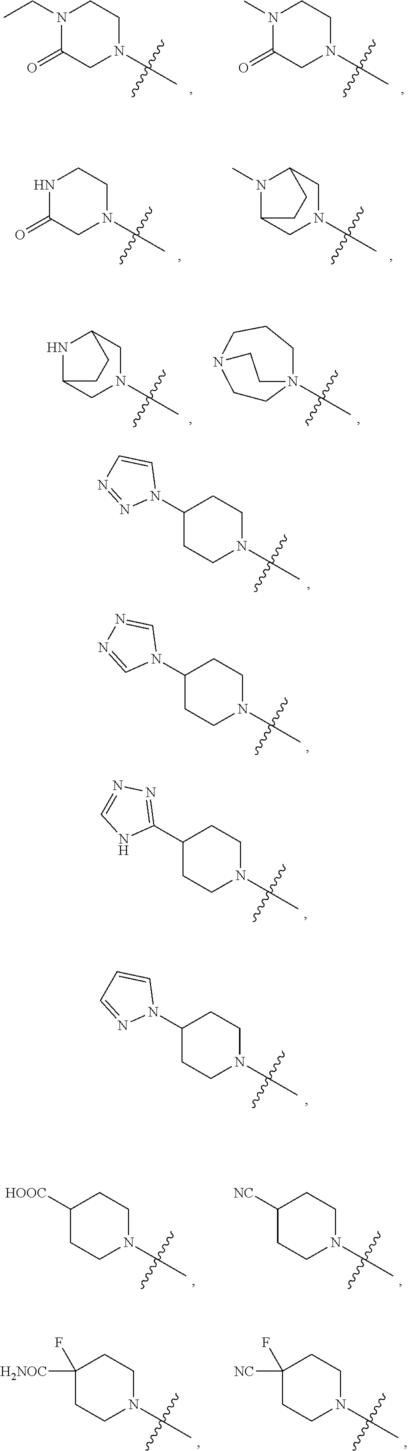

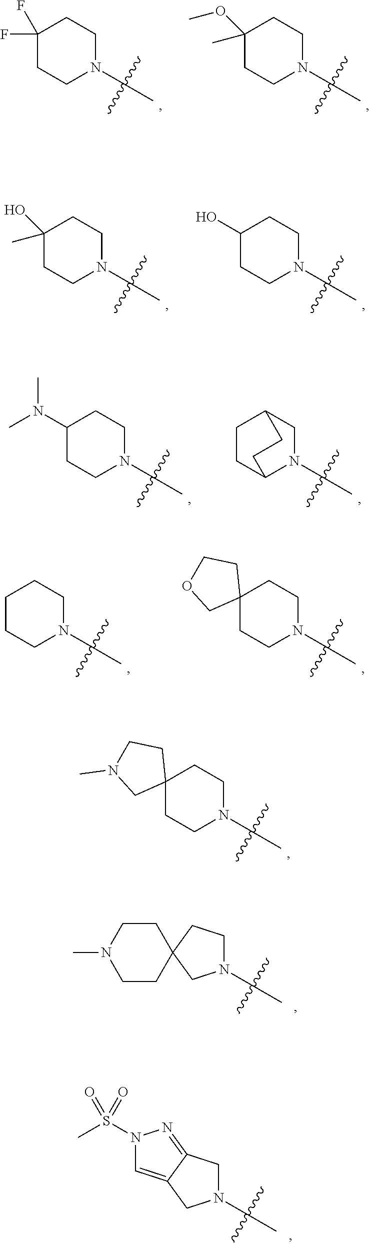

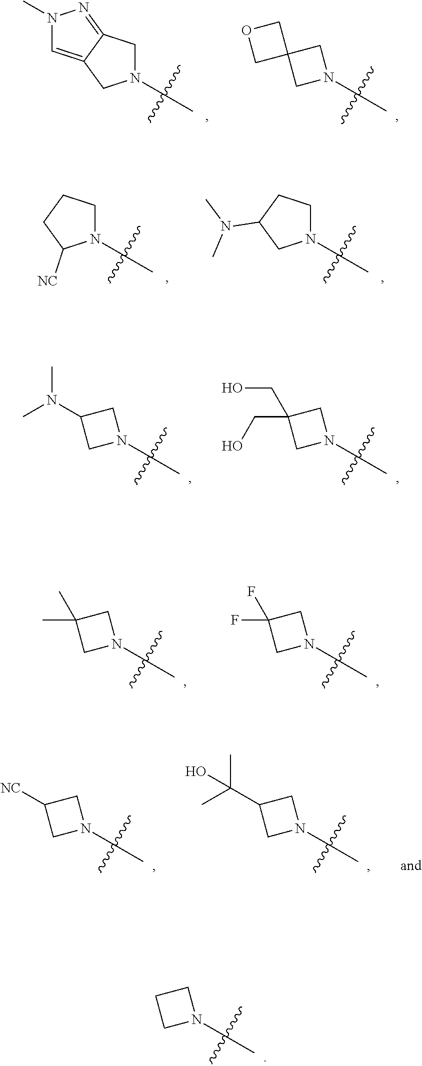

In certain embodiments of Formula V, the nitrogen-containing heterocycloalkyl is chosen from:

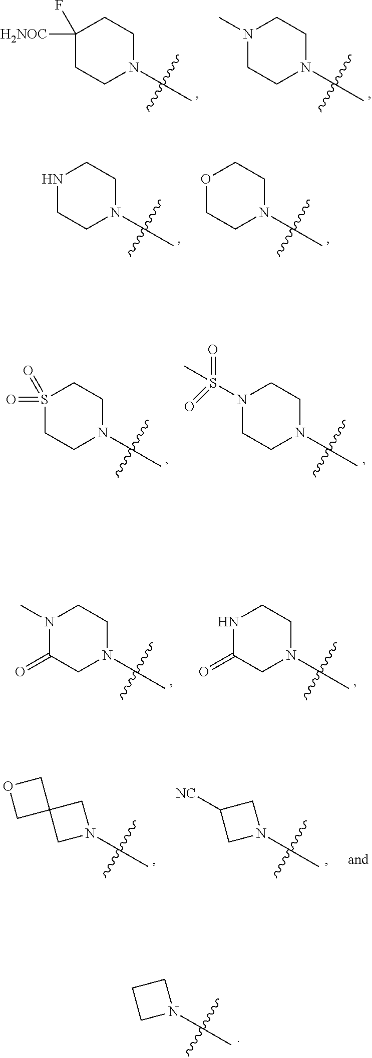

In certain embodiments of Formula V, the nitrogen-containing heterocycloalkyl is:

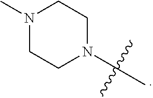 [SMILES: *N1CCN(C)CC1 ; "."]

In certain embodiments of Formula V, R^{6b} is chosen from fluoro, methoxy, and hydroxy.

In certain embodiments of Formula V, R^{6b} is fluoro.

In certain embodiments, the compound of Formula V is:

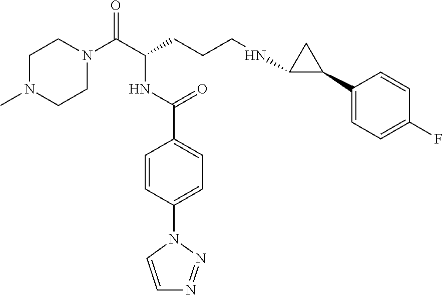 [SMILES: CN1CCN(C(=O)[C@H](CCCN[C@@H]2C[C@H]2c2ccc(F)cc2)NC(=O)c2ccc(-n3ccnn3)cc2)CC1]

or a salt, polymorph, or solvate thereof.

In certain embodiments, the compound of Formula V is a salt of the formula:

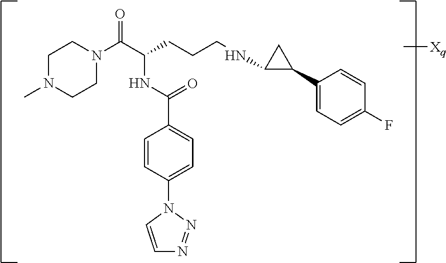

or a polymorph or solvate thereof, wherein:
X is chosen from tosylate, sulfate, tartrate, oxalate, besylate, fumarate, citric, esylate, and malate; and

q is an integer chosen from 1 and 2.

In certain embodiments, X is tosylate.

In certain embodiments, q is 2.

In certain embodiments, the compound of Formula V is

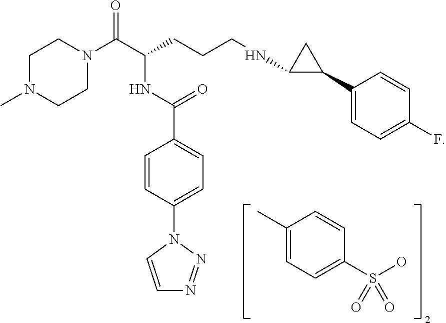

Also provided are embodiments wherein any embodiment above may be combined with any one or more of these embodiments, provided the combination is not mutually exclusive. As used herein, two embodiments are “mutually exclusive” when one is defined to be something which cannot overlap with the other. For example, an embodiment wherein Y is CH₂ is mutually exclusive with an embodiment wherein Y is NR^{4b}. However, an embodiment wherein R¹ and R² are taken together to form a nitrogen-containing heterocycloalkyl is not mutually exclusive with an embodiment wherein R⁵ is phenyl optionally substituted with fluorine.

The compounds disclosed above, or any subset or species of them, may be used in any of the methods of treatment and effecting of clinically/therapeutically relevant endpoints disclosed below in Methods of Treatment of Disease and Use in Medicaments.

In accordance with another aspect of the invention, a compound as disclosed herein is provided for use as a medicament.

In accordance with another aspect of the invention, a compound as disclosed herein is provided for use in the manufacture of a medicament for the prevention or treatment of a disease or condition, or effecting of a clinically relevant endpoint, as discussed below in Methods of Treatment of Disease and Use in Medicaments.

In accordance with another aspect of the invention, a pharmaceutical composition is provided which comprises a compound as disclosed herein, together with a pharmaceutically acceptable carrier.

In certain embodiments, the pharmaceutical composition is formulated for oral administration.

In certain embodiments, the pharmaceutical composition additionally comprises another therapeutic agent.

### Methods of Treatment of Disease and Uses in Medicaments

Provided herein are methods for treating or preventing a myeloproliferative neoplasm, the method comprising administering to a subject in need thereof an LSD1 inhibitor compound as disclosed herein. In certain embodiments, the method effects or results in one or more of the following:
suppresses proliferation of malignant myeloid cells in a subject in need thereof;
reduces reticulin and/or collagen bone marrow fibrosis in a subject in need thereof;
reduces plasma levels of one or more inflammatory cytokines in a subject in need thereof;
reduces mutant allele burden in a subject in need thereof;
reduces a pathologically elevated red blood cell mass in a subject in need thereof;
reduces the mass of malignant myeloid cells in a subject in need thereof;
reduces abnormal spleen size or volume in a subject in need thereof;
reduces the amount of extramedullary hematopoiesis in a subject in need thereof;
reduces the constitutional symptoms of myelofibrosis measured by patient-reported surveys in a subject in need thereof;
reduces platelet counts in a subject in need thereof;
reduces elevated an elevated level of bone marrow cells of granulocytic lineage in a subject in a subject in need thereof;
reduces bone marrow cellularity to age-adjusted normocellularity with fewer than 5% blast cells in a subject in need thereof;
reduces hemoglobin level in a PV patient to <160 g/L in a PV patient;
decreases red cell mass in a PV patient, wherein the decrease is inferred from hemoglobin levels Hb of <160 g/L; or
increases hemoglobin to a value >100 g/L and less than the upper limit of age- and sex adjusted normal in a MF patient.

In certain embodiments, the method effects or results in two or more of the foregoing. In certain embodiments, the method effects or results in three or more of the foregoing. In certain embodiments, the method effects or results in two or more of the foregoing other than reduces platelet counts in a subject in need thereof. In certain embodiments, the one, two, three, or more of the foregoing is limited by a recitation below in paragraphs [0294]-[0314].

In certain embodiments, the myeloproliferative neoplasm is selected from the group consisting of polycythemia vera (PV), essential thrombocythemia (ET), myelofibrosis (MF), chronic myelogenous leukemia (CML), chronic neutrophilic leukemia (CNL), and chronic eosinophilic leukemia (CEL). In certain embodiments, the myeloproliferative neoplasm is selected from the group consisting of polycythemia vera (PV), essential thrombocythemia (ET), and myelofibrosis (MF). In certain embodiments, the myeloproliferative neoplasm is myelofibrosis selected from primary myelofibrosis (PMF) and post PV/ET myelofibrosis. In certain embodiments, the myeloproliferative neoplasm is primary myelofibrosis (PMF). In certain embodiments, the myeloproliferative neoplasm is post PV/ET myelofibrosis. In certain embodiments, the myeloproliferative neoplasm is essential thrombocythemia. In certain embodiments, the myeloproliferative neoplasm is polycythemia vera. In certain embodiments, the myeloproliferative neoplasm is chronic myelogenous leukemia. In certain embodiments, the myeloproliferative neoplasm is chronic neutrophilic leukemia. In certain embodiments, the myeloproliferative neoplasm is chronic eosinophilic leukemia. In certain embodiments, the patient is a human.

Provided herein is a method for suppressing proliferation of malignant myeloid cells, in a subject in need thereof, the method comprising administering a therapeutically effective amount of an LSD1 inhibitor. In certain embodiments, the malignant myeloid cells have mutations in one of the genes selected from the group consisting of Janus Kinase 2 (JAK2), myeloproliferative leukemia virus oncogene (MPL) and calreticulin (CALR). In certain embodiments, the method further comprises the step of determining whether said subject has mutations in one of the genes selected from the group consisting of Janus Kinase 2 (JAK2), myeloproliferative leukemia virus oncogene (MPL) and calreticulin (CALR). In certain embodiments, the malignant myeloid cells are malignant stem cells. In certain embodiments, reduction of the malignant myeloid cells is measured by the frequency of the mutant allele burden as measured by PCR or sequencing or other methods known in the art. In certain embodiments, the malignant myeloid cells are reduced by at least 50%. In certain embodiments, the malignant myeloid cells are reduced by 2 or more logs (100× or more).

Provided herein is a method for reducing reticulin and/or collagen bone marrow fibrosis in a subject in need thereof, the method comprising administering a therapeutically effective amount of an LSD1 inhibitor. In certain embodiments, the bone marrow fibrosis is reticulin bone marrow fibrosis. In certain embodiments, the bone marrow fibrosis is collagen bone marrow fibrosis. In certain embodiments, the bone marrow fibrosis is reticulin and collagen bone marrow fibrosis. In certain embodiments, the reticulin and/or collagen bone marrow fibrosis is reduced by at least one grade. e.g., from 3 to 2, or from 2 to 1, or from 1 to 0. In certain embodiments, the reticulin and/or collagen bone marrow fibrosis is reduced by at least two grades. See, e.g., Arber et al.

In certain embodiments, the subject has mutations in one of the genes selected from the group consisting of Janus Kinase 2 (JAK2), myeloproliferative leukemia virus oncogene (MPL) and calreticulin (CALR). In certain embodiments, the LSD1 inhibitor is an LSD1 inhibitor compound as disclosed herein. The mutations may be assessed by methods known in the art, for example those disclosed in Spivak J, “Narrative Review: Thrombocytosis, polycythemia vera, and JAK2 mutations: the phenotypic mimicry of chronic myeloproliferation,” Annals of Internal Medicine 2010 152(5):300-306 or Zhan H and Spivak J L, “The diagnosis and management of polycythemia vera, essential thrombocythemia, and primary myelofibrosis in the JAK2 V617F era,” Clin Adv Hematol Oncol, 2009 May; 7(5):334-42.

Provided herein is a method for reducing plasma levels of one or more inflammatory cytokines in a subject in need thereof, the method comprising administering a therapeutically effective amount of an LSD1 inhibitor. In certain embodiments, one or more of the inflammatory cytokines is selected from the group consisting of interferon gamma, interleukin 6, tumor necrosis factor alpha, and interleukin 8, interleukin 12, interleukin 15, interleukin 17 and CXCL5.

In certain embodiments, the measured cytokine or cytokines are reduced to about the following levels, or below:
IL-6 is reduced to below about 9 pg/mL;
IL-8 is reduced to below about 18 pg/mL;
IL-10 is reduced to below about 51 pg/mL;
IL-12 is reduced to below about 182 pg/mL;
IL-15 is reduced to below about 38 pg/mL;
TNF-alpha is reduced to below about 15 pg/mL; and/or
INF-gamma is reduced to below about 23 pg/mL.

In certain embodiments, two, three, four, five, or more of the inflammatory cytokines are reduced.

Provided herein is a method for reducing the mass of malignant myeloid cells in a subject in need thereof, the method comprising administering a therapeutically effective amount of an LSD1 inhibitor. In certain embodiments, the mass of malignant myeloid cells is measured by flow cytometry immunophenotyping. In certain embodiments, the mass of malignant myeloid cells is measured by the frequency of the mutant allele, a ratio of the number of cells with the causative MPN mutations (MPL, CALR or JAK2) over the total number of cells that contain both the wild-type and mutant alleles.

Provided herein is a method for reducing mutant allele burden in a subject in need thereof, the method comprising a therapeutically effective amount of an LSD1 inhibitor. In certain embodiments, the mutant allele is an allele of one of the genes selected from the group consisting of Janus Kinase 2 (JAK2), myeloproliferative leukemia virus oncogene (MPL) and calreticulin (CALR). In certain embodiments, the LSD1 inhibitor is an LSD1 inhibitor compound as disclosed herein. In certain embodiments, the mutant allele burden is reduced by about 50% of a subject's (or the subject pool's average) mutant allele burden of mutated Janus Kinase 2 (JAK2), myeloproliferative leukemia virus oncogene (MPL) or calreticulin (CALR). In certain embodiments, the reduction in mutant allele burden is measured within patient(s) after treatment and compared to the level prior to treatment to the level after a course of treatment. In certain embodiments, the mutant allele burden is reduced to a level where mutant alleles of Janus Kinase 2 (JAK2), myeloproliferative leukemia virus oncogene (MPL) and calreticulin (CALR) are undetectable. Mutant allele burden may be assessed by methods known in the art, including those disclosed above.

Provided herein is a method for reducing a pathologically elevated red blood cell mass in a subject in need thereof, the method comprising administering a therapeutically effective amount of an LSD1 inhibitor. In certain embodiments, the subject has polycythemia vera. In certain embodiments, the subject has a mutation in Janus Kinase 2 (JAK2). In certain embodiments, the elevated red blood cell mass is inferred by the measure of the hematocrit or blood hemoglobin. In certain embodiments, measured the hematocrit or the hemoglobin should be reduced to the normal range appropriate to gender. For example, in certain embodiments:
blood hemoglobin will be reduced to less than 16.5 g/dL for a male PV patient or to less than 16.0 g/dL for a female PV patient;
hematocrit will be reduced to less than 49% for a male PV patient or to less than 48% for a female PV patient.

In certain embodiments, the elevated red blood cell mass is measured by isotopic red cell mass measurement. In certain embodiments the increased red cell mass is greater than 25% above mean normal predicted value.

Provided herein is a method for reducing an elevated white blood cell count in a subject in need thereof, the method comprising administering a therapeutically effective amount of an LSD1 inhibitor. In certain embodiments, subject has chronic neutrophilic leukemia.

Also provided herein is a method for reducing an elevated level of bone marrow cells of granulocytic lineage in a subject in need thereof, the method comprising administering a therapeutically effective amount of an LSD1 inhibitor. In certain embodiments, the bone marrow cells of granulocytic lineage are reduced to a value within the normal range. Also provided herein is a method for, in a subject in need thereof, reducing bone marrow cellularity to age-adjusted normocellularity with fewer than 5% blast cells, the method comprising administering a therapeutically effective amount of an LSD1 inhibitor. In certain embodiments, subject has chronic neutrophilic leukemia.

Provided herein is a method for increasing hemoglobin to >100 g/L up to a level less than the upper limit of age- and sex adjusted normal in a subject in need thereof, the method comprising administering a therapeutically effective amount of an LSD1 inhibitor.

Also provided is a method for a) reducing hemoglobin level in a PV patient to <160 g/L, or b) decreasing red cell mass in a PV patient, wherein the decrease is inferred from hemoglobin levels Hb of <160 g/L, either comprising administering a therapeutically effective amount of an LSD1 inhibitor. Also provided is a method for increasing hemoglobin to >100 g/L in a MF patient, comprising administering a therapeutically effective amount of an LSD1 inhibitor. Also provided is a method for increasing hemoglobin to a value >100 g/L and less than the upper limit of age- and sex adjusted normal in a MF patient, the method comprising administering a therapeutically effective amount of an LSD1 inhibitor. In certain embodiments, said subject has a mutation in one of the genes selected from the group consisting of Janus Kinase 2 (JAK2), myeloproliferative leukemia virus oncogene (MPL) and calreticulin (CALR). In certain embodiments, said subject has essential thrombocythemia. In certain embodiments, the transfusion burden of said patient is reduced.

Provided herein is a method for reducing abnormal spleen size or volume in a subject in need thereof, the method comprising administering a therapeutically effective amount of an LSD1 inhibitor. In certain embodiments, said subject has a mutation in one of the genes selected from the group consisting of Janus Kinase 2 (JAK2), myeloproliferative leukemia virus oncogene (MPL) and calreticulin (CALR).

Provided herein is a method for reducing the amount of extramedullary hematopoiesis in a subject in need thereof, the method comprising administering a therapeutically effective amount of an LSD1 inhibitor. In certain embodiments, said subject has a mutation in one of the genes selected from the group consisting of Janus Kinase 2 (JAK2), myeloproliferative leukemia virus oncogene (MPL) and calreticulin (CALR). In certain embodiments, the amount of extramedullary hematopoiesis is measured by splenomegaly. In certain embodiments, splenomegaly in said subject is reduced by at least about 30%, at least about 35%, at least about 40%, or least about 45%. In certain embodiments, splenomegaly in said subject is reduced by at least 35%. In certain embodiments, splenomegaly in is reduced by at least 35% in about 50% of patients.

Provided herein is a method for reducing the constitutional symptoms of myelofibrosis, as measured by patient-reported surveys in a subject in need thereof, the method comprising administering a therapeutically effective amount of an LSD1 inhibitor. In certain embodiments, said constitutional symptoms comprise one or more symptoms selected from the group consisting of fatigue, early satiety, abdominal discomfort, inactivity, problems with concentration, numbness and/or tingling in the hands and feet, night sweats, pruritis, bone pain, fever greater than 100° F., and unintentional weight loss.

In certain embodiments, said patient-reported survey is the Myeloproliferative Neoplasm Symptom Assessment Form (MPN-SAF). The MPN-SAF is a validated clinical assessment form for the most common symptoms of myeloproliferative neoplasms, in which patients self-reports their score, on a scale of 1-10, of various common symptoms, where 1 is the most favorable or the symptom is absent, and 10 is the least favorable or the symptom is the worst imaginable. See, e.g., Scherber R et al., The Myeloproliferative Neoplasm Symptom Assessment Form (MPN-SAF): International Prospective Validation and Reliability Trial in 402 patients,” Blood 118(2):401-08 (2014). Either the full or abbreviated forms may be administered to the patient. In the abbreviated version, a “total symptom score” (TSS) may be calculated from the ten most clinically relevant symptoms from the 17-item MPN-SAF: worst fatigue, concentration, early satiety, inactivity, night sweats, itching, bone pain, abdominal discomfort, weight loss, and fever. The MPN-SAF TSS thus has a possible range of 0 to 100. Quality of life scores are defined as “clinically deficient” when they rate as at least 4 of 10; “moderate” if symptoms are rated as ≥4 of 10 or ≤6 of 10; and “severe” if symptoms are rated as ≥7 of 10. For patients who complete at least six of these 10 items on the BFI and MPN-SAF, the MPN TSS is computed as the average of the observed items multiplied by 10 to achieve a 0-to-100 scale. See, e.g., Emanuel R M et al., “Myeloproliferative neoplasm (MPN) symptom assessment form total symptom score: prospective international assessment of an abbreviated symptom burden scoring system among patients with MPNs,” J Clin Oncol 30(33):4098-103 (2012).

In certain embodiments, the total symptom score (MPN-SAF:TSS) is reduced by at least 50%.

In certain embodiments, said patient-reported survey is the Myelofibrosis Symptom Assessment Form (MF-SAF). See. e.g., Mesa R A et al., “The Myelofibrosis Symptom Assessment Form (MFSAF): an evidence-based brief inventory to measure quality of life and symptomatic response to treatment in myelofibrosis,” Leuk Res. 33(9): 1199-203 (2009). In certain embodiments, the MF-SAF total symptom score is reduced by at least 50%.

Also provided herein are further embodiments of any of the above embodiments e.g. in paragraphs [0292]-[0312], wherein:
the subject has a mutation in one of the genes selected from the group consisting of Janus Kinase 2 (JAK2), myeloproliferative leukemia virus oncogene (MPL) and calreticulin (CALR);
the subject has a myeloproliferative neoplasm;
the subject has a myeloproliferative neoplasm selected from the group consisting of polycythemia vera (PV), essential thrombocythemia (ET), and myelofibrosis;
the subject has myelofibrosis;
the subject has myelofibrosis selected from primary myelofibrosis (PMF) and post PV/ET myelofibrosis;
the subject has post PV/ET myelofibrosis (MF);
the subject has primary myelofibrosis (PMF);
the subject has polycythemia vera;
the subject has essential thrombocythemia;
the subject has chronic myelogenous leukemia;
the subject has chronic neutrophilic leukemia; or
the subject has chronic eosinophilic leukemia;
and/or the subject is a human;
and/or the LSD1 inhibitor is an LSD1 inhibitor compound as disclosed herein.

Also provided are embodiments wherein any method embodiment above may be combined with any one or more of these embodiments, provided the combination is not mutually exclusive. As used herein, two embodiments are “mutually exclusive” when one is defined to be something which cannot overlap with the other. For example, an embodiment wherein the disorder to be treated is primary myelofibrosis (PMF) is mutually exclusive with an embodiment wherein the disorder to be treated is post PV/ET myelofibrosis (MF), because these classifications are the product of different diagnoses. However, an embodiment wherein the disorder to be treated is PMF is not mutually exclusive with an embodiment wherein reticulin and/or collagen bone marrow fibrosis is reduced, because reticulin and/or collagen bone marrow fibrosis occur in PMF.

The methods disclosed above, or any subset or species of them, may use any of the compounds disclosed above as LSD1 inhibitors, either a discrete chemical species or as described by one of the formulae or embodiments, or a pharmaceutical composition comprising them.

### Biological Assays

Compounds disclosed herein have been shown to be inhibitors of LSD1, as disclosed for example in WO 2015/021128 and WO 2016/130952, or any of the references cited above, the contents of which are hereby incorporated by reference.

Compounds were evaluated in a mouse model of thrombocytosis and myelofibrosis, as well as a knock-in mouse model of myeloproliferative neoplasms wherein animals carried the JAK2^{V617F} mutation.

### MPL^{W515L}-Driven Model of Thrombocytosis and Myelofibrosis

Compound 1 was tested in the murine model of MPLW515L-induced thrombocytosis and myelofibrosis as described by Koppikar et al., Blood, 2010, 115, 2919-2127. Briefly, bone marrow cells from 5FU-treated male donors were harvested and transduced with viral supernatants containing either MSCV-hMPL^{W515L}-GFP or MSCV-hMPL^{WT}-GFP, and 750,000 bone marrow cells of each type were injected into the tail veins of lethally irradiated female BALB/c mice. Disease severity was assessed in all mice by nonlethal bleeds performed 11 days after transplantation. MPL^{W515L} mice were then randomized for once-a-day treatment with Compound 1 (5 mg/kg/day) or vehicle by oral gavage, for 28 days beginning 12 days after transplantation. WT mice were randomized to receive either Compound 1 or vehicle. Except for mice killed for flow cytometry, mice were treated for 28 days, or until criteria were met for killing based on lethargy, loss of body weight, and palpable splenomegaly.

Cytokine levels were assessed in mouse serum as described by Koppikar et al. Peripheral blood analyses for white blood cells and platelets were performed as in Koppikar et al. The presence of GFP⁺ cells in peripheral blood was detected by fluorescence-activated cell sorter (FACS) analysis as described by Koppikar et al. Pathologic analysis by immunohistochemistry was performed as described by Koppikar et al.

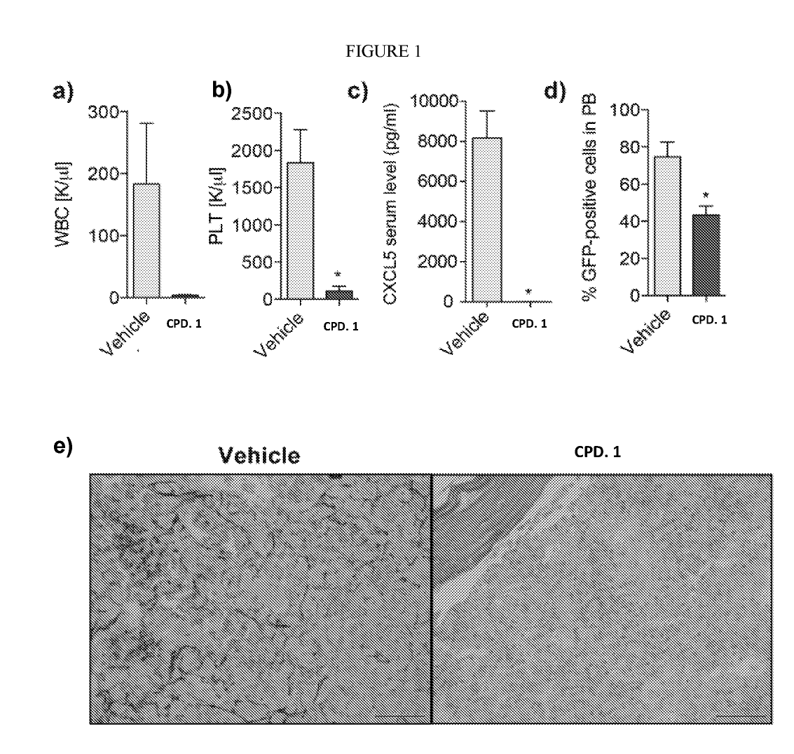

Treatment with LSD1 inhibitor Compound 1 caused significant reduction in white blood cells (FIG. 1a) and platelet counts (FIG. 1b) as compared to vehicle-treated controls, in diseased animals. Pathologic analysis of bone marrow and spleen confirmed a marked reduction in myeloproliferation as well as a reversal of extramedullary hematopoiesis (EMH). Most notably, there was observed a marked reduction in reticulin and collagen fibrosis with Compound 1 treatment (FIG. 1b). Spleen weights were also reduced in drug-treated animals.

There was also observed a significant reduction in the secretion of the inflammatory cytokine Cxcl5 (FIG. 1c), a key participant in pathologic inflammatory states, in animals receiving Compound 1. Treatment with Compound 1 also resulted in significantly reduced mutant allele burden as compared to vehicle-treated animals (FIG. 1d). Whereas 74.6% of circulating cells in mice treated with vehicle were GFP-positive cells, only 43.2% of circulating cells were GFP⁺ in Compound 1-treated mice (FIG. 1d). Flow cytometry analysis of spleen and BM revealed reduced numbers of CD11 b/Gr1-positive myeloid cells and CD41-positive megakaryocytes. The numbers of mutant GFP-positive myeloid cells and megakaryocytes in these tissues were also significantly reduced by Compound 1 treatment.

JAK2^{V617F} Knock-in mouse model

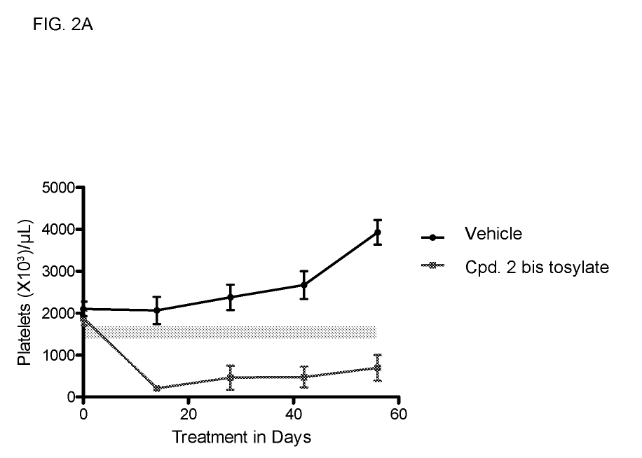

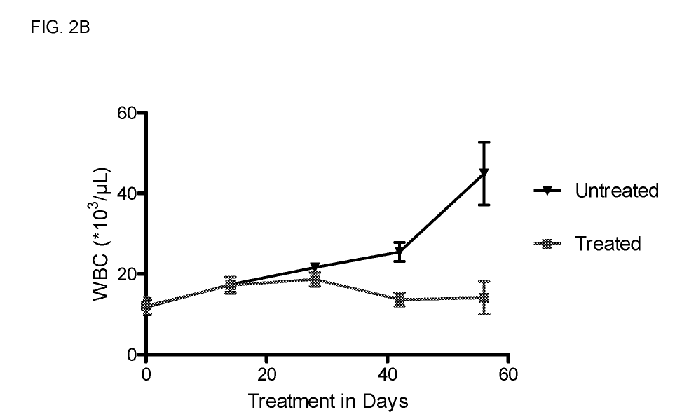

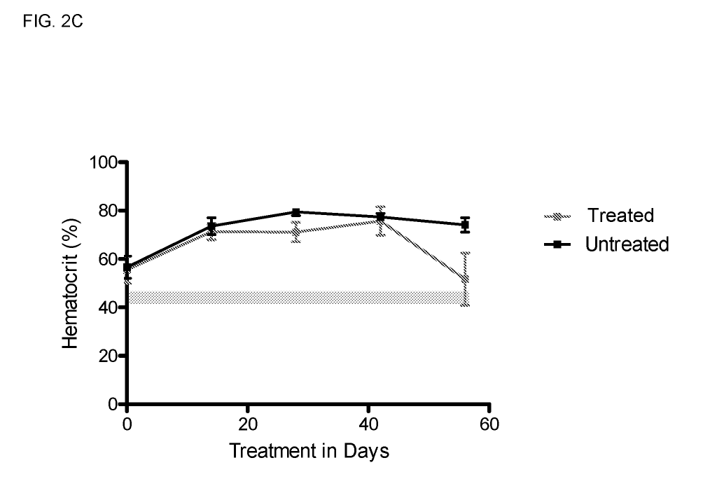

Compound [IMG 7289; need number or name inserted] was evaluated in the JAK2^{V617F} knock-in mouse model of myeloproliferative neoplasm (MPN). The procedure of Hasan et al. Blood, 2013, 122, 1464-1477, was followed. (See also Mullally et al. Cancer Cell 2010, 17, 584-596.) Animals bearing the JAK2^{V617F} were treated with Compound 2 tosylate (25 mg/kg/day) or vehicle for a total of 56 days. Blood samples were taken at the indicated time points and analyzed for hematocrit, white blood cell count (WBC) and platelets as described in Hasan et al. Non-drug-treated animals expressing the JAK2^{V617F} mutation manifested elevated levels of platelets, WBCs, and hematocrit as compared to wild-type animals. Treatment with Compound 2 tosylate resulted in reduction of platelet levels (FIG. 2a), white blood cell count (FIG. 2b), and hematocrit (FIG. 2c).

From the foregoing description, one skilled in the art can easily ascertain the essential characteristics of this invention, and without departing from the spirit and scope thereof, can make various changes and modifications of the invention to adapt it to various usages and conditions.

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinence of the cited references.

What is claimed is:

1. A method for treating or preventing a myeloproliferative neoplasm, the method comprising administering to a subject in need thereof a therapeutically effective amount of an LSD1 inhibitor.

2. The method as recited in claim 1, wherein the myeloproliferative neoplasm is selected from the group consisting of polycythemia vera (PV), essential thrombocythemia (ET), and myelofibrosis.

3. The method as recited in claim 2, wherein the myeloproliferative neoplasm is myelofibrosis selected from primary myelofibrosis (PMF) and post PV/ET myelofibrosis (MF).

4. The method as recited in claim 3, wherein the myeloproliferative neoplasm is post PV/ET myelofibrosis (MF).

5. The method as recited in claim 2, wherein the myeloproliferative neoplasm is polycythemia vera.

6. The method as recited in claim 2, wherein the myeloproliferative neoplasm is essential thrombocythemia.

7. The method as recited in claim 1, wherein said treatment with a compound of Formula I results in suppression of proliferation of malignant myeloid cells.

8. The method as recited in claim 1, wherein said treatment with a compound of Formula I results in a reduction in reticulin and collagen bone marrow fibrosis.

9. The method as recited in claim 1, wherein said treatment with a compound of Formula I results in a reduction in the plasma levels of one or more inflammatory cytokines.

10. The method as recited in claim 9, wherein said treatment with a compound of Formula I results in a reduction in the plasma levels of one or more cytokines selected from the group consisting of CXCL5, interferon gamma, interleukin 6, interleukin 12, interleukin 15, tumor necrosis factor alpha, and interleukin 8.

11. The method as recited in claim 1, wherein said treatment with a compound of Formula I results in a reduction of mutant allele burden.

12. A method for suppressing proliferation of malignant myeloid cells in a subject in need thereof, the method comprising administering a therapeutically effective amount of an LSD1 inhibitor.

13. The method as recited in claim 12, wherein the malignant myeloid cells have mutations in one more genes selected from the group consisting of Janus Kinase 2 (JAK2), myeloproliferative leukemia virus oncogene (MPL) and calreticulin (CALR).

14. The method as recited in claim 12, further comprising the step of determining whether said subject has mutations in one of the genes selected from the group consisting of Janus Kinase 2 (JAK2), myeloproliferative leukemia virus oncogene (MPL) and calreticulin (CALR).

15. A method for reducing reticulin and collagen bone marrow fibrosis in a subject in need thereof, the method comprising administering a therapeutically effective amount of an LSD1 inhibitor.

16. The method as recited in claim 15, wherein said subject has myelofibrosis.

17. The method as recited in claim 16, wherein said subject has mutations in one of the genes selected from the group consisting of Janus Kinase 2 (JAK2), myeloproliferative leukemia virus oncogene (MPL) and calreticulin (CALR).

18. A method for reducing plasma levels of one or more inflammatory cytokines in a subject in need thereof, the method comprising administering a therapeutically effective amount of an LSD1 inhibitor.

19. The method as recited in claim 18, wherein the one or more inflammatory cytokines is one or more cytokines selected from the group consisting of interferon gamma, interleukin 6, interleukin 12, interleukin 15, tumor necrosis factor alpha, and interleukin 8 and CXCL5.

20. A method for reducing mutant allele burden in a subject in need thereof, the method comprising a therapeutically effective amount of an LSD1 inhibitor.

21. The method as recited in claim 20, wherein said mutant allele is an allele of one of the genes selected from the group consisting of Janus Kinase 2 (JAK2), myeloproliferative leukemia virus oncogene (MPL) and calreticulin (CALR).

22. A method for reducing a pathologically elevated red blood cell mass in a subject in need thereof, the method comprising administering a therapeutically effective amount of an LSD1 inhibitor.

23. The method as recited in claim 22, wherein said subject has polycythemia vera.

24. The method as recited in claim 22, wherein said subject has mutations in one of the genes selected from the group consisting of Janus Kinase 2 (JAK2), myeloproliferative leukemia virus oncogene (MPL) and calreticulin (CALR)

25. The method as recited in claim 22, wherein the elevated blood cell mass is measured as hematocrit or blood hemoglobin.

26. The method as recited in claim 22, wherein measured blood hemoglobin has a value greater than 16.5 g/dL for a male subject or greater than 16.0 g/dL for a female subject.

27. The method as recited in claim 22, wherein measured hematocrit is greater than 49% for a male subject or greater than 48% for a female subject.

28. The method as recited in claim 22, wherein the elevated blood cell mass is measured by isotopic red cell mass measurement.

29. The method as recited in claim 22, wherein the increased red cell mass is greater than 25% above mean normal predicted value.

30. A method for reducing an elevated level of bone marrow cells of granulocytic lineage in a subject in a subject in need thereof, the method comprising administering a therapeutically effective amount of an LSD1 inhibitor.

31. The method as recited in claim 30, wherein said subject has chronic neutrophilic leukemia.

32. The method as recited in claim 30, wherein said subject has a mutation in one of the genes selected from the group consisting of Janus Kinase 2 (JAK2), myeloproliferative leukemia virus oncogene (MPL) and calreticulin (CALR).

33. A method for reducing the mass of malignant myeloid cells in a subject in need thereof, the method comprising administering a therapeutically effective amount of an LSD1 inhibitor.

34. The method as recited in claim 33, wherein the mass of malignant myeloid cells is measured by flow cytometry immunophenotyping.

35. The method as recited in claim 33, wherein the mass of malignant myeloid cells is measured by the frequency of the mutant allele, a ratio of the number of cells with the causative MPN mutations (MPL, CALR or JAK2) over the total number of cells that contain both the wild-type and mutant alleles.

36. A method for reducing abnormal spleen size or volume in a subject in need thereof, the method comprising administering a therapeutically effective amount of an LSD1 inhibitor.

37. The method as recited in claim 36, wherein said subject has myelofibrosis.

38. The method as recited in claim 36, wherein said subject has a mutation in one of the genes selected from the group consisting of Janus Kinase 2 (JAK2), myeloproliferative leukemia virus oncogene (MPL) and calreticulin (CALR).

39. A method for reducing the amount of extramedullary hematopoiesis in a subject in need thereof, the method comprising administering a therapeutically effective amount of an LSD1 inhibitor.

40. The method as recited in claim 39, wherein said subject has a mutation in one of the genes selected from the group consisting of Janus Kinase 2 (JAK2), myeloproliferative leukemia virus oncogene (MPL) and calreticulin (CALR).

41. The method as recited in claim 39, wherein the amount of extramedullary hematopoiesis is measured by splenomegaly.

42. The method as recited in claim 41, wherein splenomegaly in said subject is reduced by at least 35%.

43. A method for reducing the constitutional symptoms of myelofibrosis measured by patient-reported surveys in a subject in need thereof, the method comprising administering a therapeutically effective amount of an LSD1 inhibitor.

44. The method as recited in claim 43, wherein the LSD1 inhibitor is a compound of claim 1.

45. The method as recited in claim 43, wherein said subject has a mutation in one of the genes selected from the group consisting of Janus Kinase 2 (JAK2), myeloproliferative leukemia virus oncogene (MPL) and calreticulin (CALR).

46. The method as recited in claim 43, wherein said constitutional symptoms comprise one or more symptoms selected from the group consisting of fatigue, early satiety, abdominal discomfort, inactivity, problems with concentration, numbness and/or tingling in the hands and feet, night sweats, pruritis, bone pain, fever greater than 100° F., and unintentional weight loss.

47. The method as recited in claim 43, wherein said patient-reported survey is the Myeloproliferative Neoplasm Assessment Form Total Symptom Score (MPN-SAF:TSS).

48. The method as recited in claim 47, wherein one or more symptoms is reduced by at least 50% in its ranking on the MPN-SAF:TSS score.

49. A method for reducing platelet count in a subject in need thereof, the method comprising administering a therapeutically effective amount of an LSD1 inhibitor.

50. The method as recited in claim 49, wherein said subject has a mutation in one of the genes selected from the group consisting of Janus Kinase 2 (JAK2), myeloproliferative leukemia virus oncogene (MPL) and calreticulin (CALR).

51. The method as recited in claim 49, wherein said subject has essential thrombocythemia.

52. The method as recited in claim 49, wherein the transfusion burden of said patient is reduced.

53. The method as recited in any one of claims 1-52 wherein the compound is a compound of Formula I: 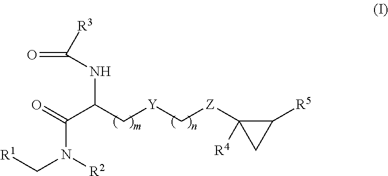
or a salt thereof, wherein:
Y is chosen from a bond, NR^{4a}, O, C(O)NH, NHC(O), S, SO₂, and CH₂;
Z is chosen from a bond, NR^{4b}, O, C(O)NH, NHC(O), S, SO₂, and CH₂;
m is an integer from 0 to 5;
n is an integer from 0 to 3;
R¹ and R² are each independently chosen from, alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;
R⁴, R^{4a}, and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
each R⁶ is independently chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, and lower alkyl; or R⁷ and R⁸ may be taken together to form a nitrogen-containing heterocycloalkyl or heteroaryl ring, which may be optionally substituted with lower alkyl;
with the proviso that when Y═CH₂, R⁴═H, and Z═R^{4b}, then m+n≠3.

54. The method as recited in claim 53, wherein Z is NR^{4b}.

55. The method as recited in claim 54, wherein R^{4b} is chosen from methyl and hydrogen.

56. The method as recited in claim 55, wherein R^{4b} is hydrogen.

57. The method as recited in claim 53, wherein the alkyl, whether by itself or as a named part of another non-cyclic substituent, is C₁-C₈ alkyl.

58. The method as recited in claim 53, wherein R³ is chosen from aryl, arylalkyl, heteroaryl, and heteroarylalkyl, any of which may be optionally substituted with between 0 and 3 R⁶ groups.

59. The method as recited in claim 6, wherein R³ is chosen from aryl and heteroaryl, any of which may be optionally substituted with between 0 and 3 R⁶ groups.

60. The method as recited in claim 53, wherein m is an integer from 0 to 1; Y is chosen from NR^{4a}, O, S, SO₂, and CH₂; n is an integer from 1 to 3; and R^{4a} is chosen from hydrogen and alkyl.

61. The method as recited in claim 60, wherein m is 0; Y is CH₂; and n is chosen from 1 and 2.

62. The compound as recited in claim 61, wherein n is 2.

63. The method as recited in claim 53, wherein R³ is 5-6 membered monocyclic or 8-12 membered bicyclic heteroaryl, in which between one and five ring members may be heteroatoms chosen from N, O, and S, and which may be optionally substituted with between 0 and 3 R⁶ groups.

64. The method as recited in claim 63, wherein R³ is 5-6 membered monocyclic heteroaryl, in which between one and four ring members may be heteroatoms chosen from N, O, and S, and which may be optionally substituted with between 0 and 3 R⁶ groups.

65. The method as recited in claim 64, wherein each R⁶ is chosen from the group consisting of lower alkyl, halogen, lower alkoxy, OCF₃ and CF₃.

66. The method as recited in claim 64, wherein R³ is chosen from

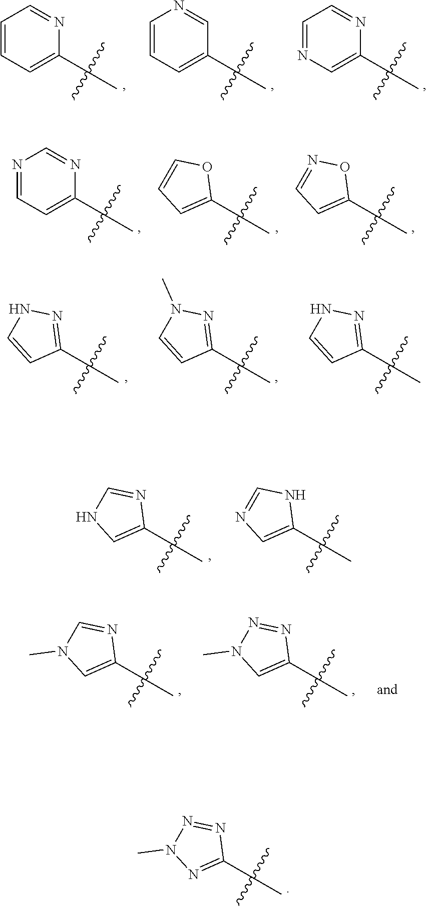

67. The method as recited in claim 53, wherein R⁴ is hydrogen.

68. The method as recited in claim 53, wherein R⁴ is methyl.

69. The method as recited in claim 53, wherein the nitrogen-containing heterocycloalkyl or heteroaryl ring formed by R¹ and R² together with the nitrogen to which they are attached contains 3 to 8 atoms.

70. The method as recited in claim 69, wherein R¹ and R² are taken together to form a nitrogen-containing heterocycloalkyl, which may be optionally substituted with between 0 and 3 R⁶ groups.

71. The method as recited in claim 70, wherein the nitrogen-containing heterocycloalkyl formed by R¹ and R² together with the nitrogen to which they are attached is chosen from:

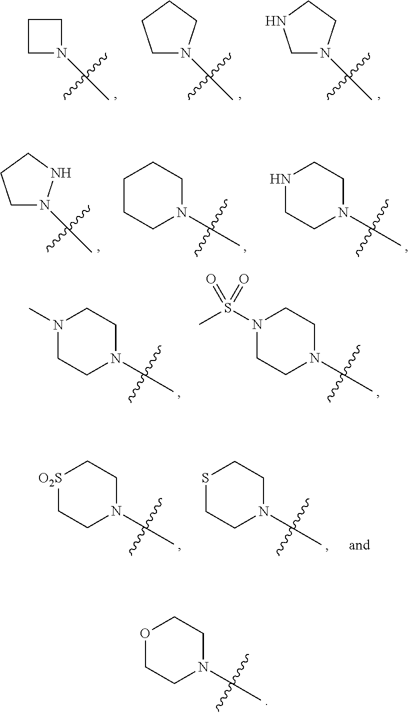

72. The method as recited in claim 70, wherein the nitrogen-containing heterocycloalkyl formed by R¹ and R² together with the nitrogen to which they are attached is chosen from:

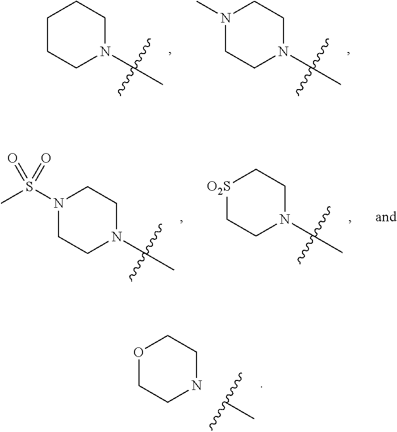

73. The method as recited in claim 70, wherein the nitrogen-containing heterocycloalkyl formed by R¹ and R² together with the nitrogen to which they are attached is

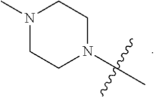 [SMILES: *N1CCN(C)CC1]

74. The method as recited in claim 69, wherein R¹ and R² are taken together to form a nitrogen-containing heteroaryl, which may be optionally substituted with between 0 and 3 R⁶ groups.

75. The method as recited in claim 74, wherein the nitrogen-containing heteroaryl formed by R¹ and R² together with the nitrogen to which they are attached is selected from the group consisting of pyrrole, imidazole, and pyrazole.

76. The method as recited in claim 69, wherein n is 2.

77. The method as recited in claim 53, wherein R⁵ is aryl, which may be optionally substituted with between 0 and 3 R⁶ groups.

78. The method as recited in claim 77, wherein R⁵ is phenyl, which may be optionally substituted with between 0 and 3 R⁶ groups.

79. The method as recited in claim 77, wherein n is 2.

80. The method of claim 77, wherein each R⁶ is independently chosen from the group consisting of lower alkyl, halogen, lower alkoxy, OCF₃ and CF₃

81. The method as recited in claim 53, wherein R⁵ is heteroaryl, which may be optionally substituted with between 0 and 3 R⁶ groups.

82. The method as recited in claim 81, wherein R⁵ is a 5-6 membered monocyclic or 8-12 membered bicyclic heteroaryl, in which between one and five ring members may be heteroatoms chosen from N, O, and S, and which may be optionally substituted with between 0 and 3 R⁶ groups.

83. The method as recited in claim 82, wherein R⁵ is a 5-6 membered monocyclic heteroaryl, in which between one and five ring members may be heteroatoms chosen from N, O, and S, and which may be optionally substituted with between 1 or 2 R⁶ groups.

84. The method as recited in claim 83, wherein R⁵ is chosen from

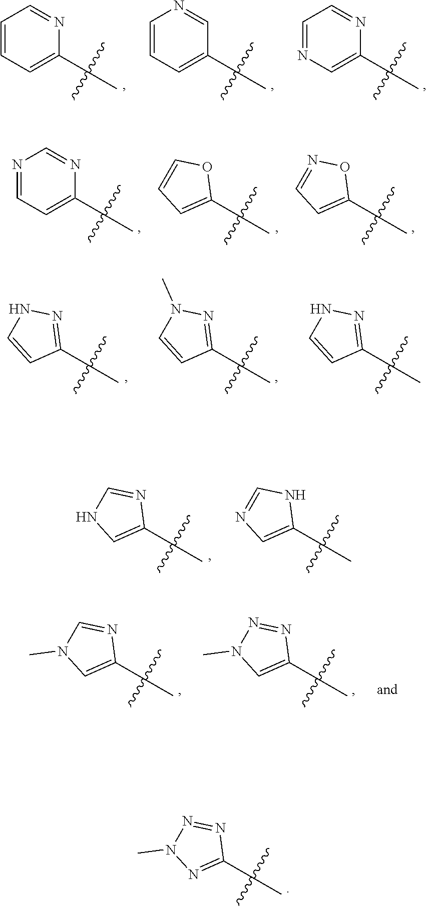

85. The method as recited in claim 81, wherein n is 2.

86. The method as recited in claim 81, wherein each R⁶ is independently chosen from the group consisting of lower alkyl, halogen, lower alkoxy, OCF₃ and CF₃.

87. The method as recited in claim 53, wherein R³ is aryl, optionally substituted with between 0 and 3 R⁶ groups.

88. The method as recited in claim 87, wherein R³ is chosen from phenyl and biphenyl, either of which may be optionally substituted with between 0 and 3 R⁶ groups.

89. The method as recited in claim 88, wherein each R⁶ is independently chosen from the group consisting of lower alkyl, halogen, lower alkoxy, OCF₃, CF₃, and heteroaryl.

90. The method as recited in claim 53, wherein the compound of Formula I is chosen from the group consisting of the Examples disclosed herein.

91. The method as recited in any one of claims 1-52 wherein the compound is a compound of Formula II: 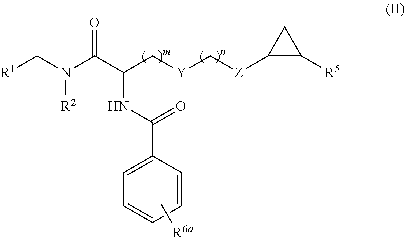
or a salt, polymorph, or solvate thereof, wherein:
Y is chosen from a bond, NR^{4a}, O, C(O)NH, NHC(O), S, SO₂, CHOH, and CH₂;
Z is chosen from a bond, NR^{4b}, O, C(O)NH, NHC(O), S, SO₂, and CH₂;
m is chosen from 0, 1, 2, 3, 4, and 5;
n is chosen from 0, 1, 2, and 3;
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4a} and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
R^{6a} is chosen from heteroaryl, cyano, and S(O)₂N(CH₃)₂;
each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;
with the proviso that when Y═CH₂ and Z═R^{4b}, then m+n≠3.

92. The method as recited in claim 91, wherein Z is NR^{4b}.

93. The method as recited in claim 92, wherein R^{4b} is chosen from methyl and hydrogen.

94. The method as recited in claim 93, wherein R^{4b} is hydrogen.

95. The method as recited in claim 94, wherein the alkyl, whether by itself or as a named part of another non-cyclic substituent, is C₁-C₈ alkyl.

96. The method as recited in claim 95, wherein m is 0; Y is CH₂; and n is chosen from 0, 1, and 2.

97. The method as recited in claim 96, wherein n is 2.

98. The method as recited in claim 95, wherein R¹ and R² are each independently chosen from alkyl, aminoalkyl, alkylsulfonylalkyl, alkoxyalkyl, and heteroaryl, and R¹ and R², together with the nitrogen to which they attach, form a nitrogen-containing heterocycloalkyl or heteroaryl ring, which may be optionally substituted with between 0 and 3 R⁶ groups

99. The method as recited in claim 98, wherein the nitrogen-containing heterocycloalkyl or heteroaryl ring formed by R¹ and R² together with the nitrogen to which they are attached contains 3 to eight atoms.

100. The method as recited in claim 99, wherein R¹ and R² are taken together to form a nitrogen-containing heterocycloalkyl, which may be optionally substituted with between 0 and 3 R⁶ groups.

101. The method as recited in claim 100, wherein the nitrogen-containing heterocycloalkyl is optionally substituted with between 0 and 3 R⁶ groups chosen from alkyl, halogen, CONH₂, SO₂CH³, cyano, spiro-heterocycloalkyl, and oxo.

102. The method as recited in claim 100, wherein the nitrogen-containing heterocycloalkyl is chosen from:

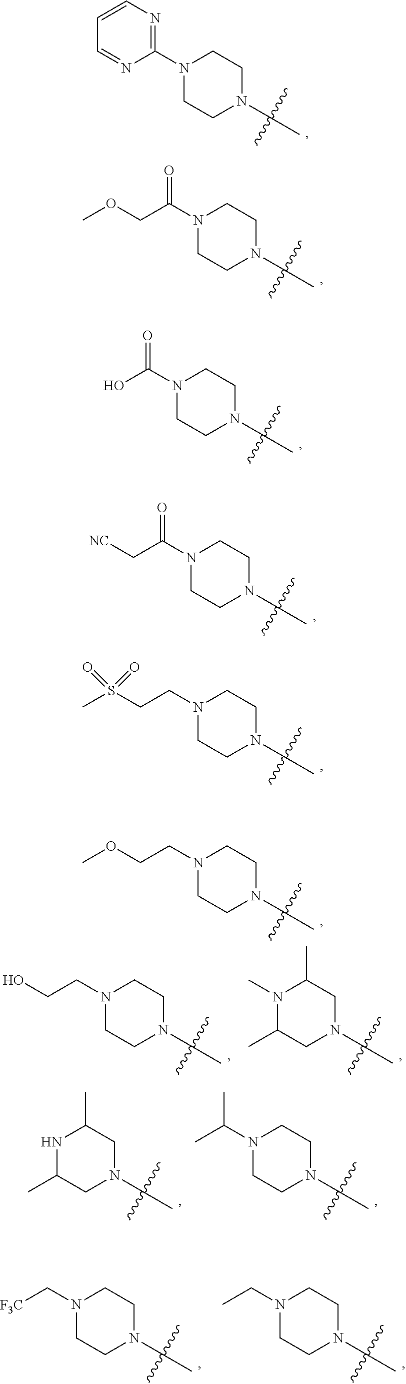

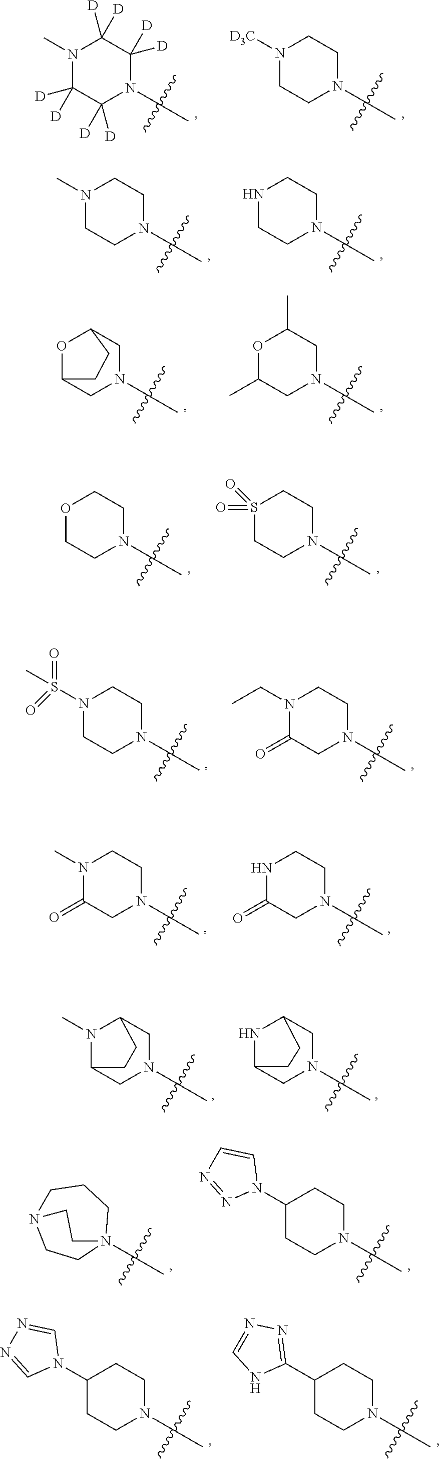

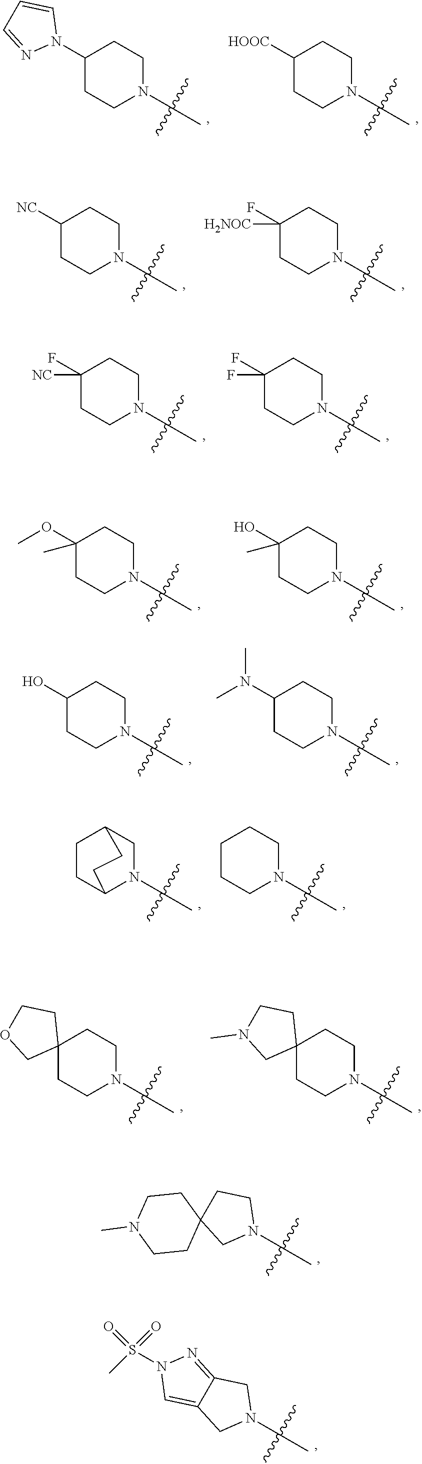

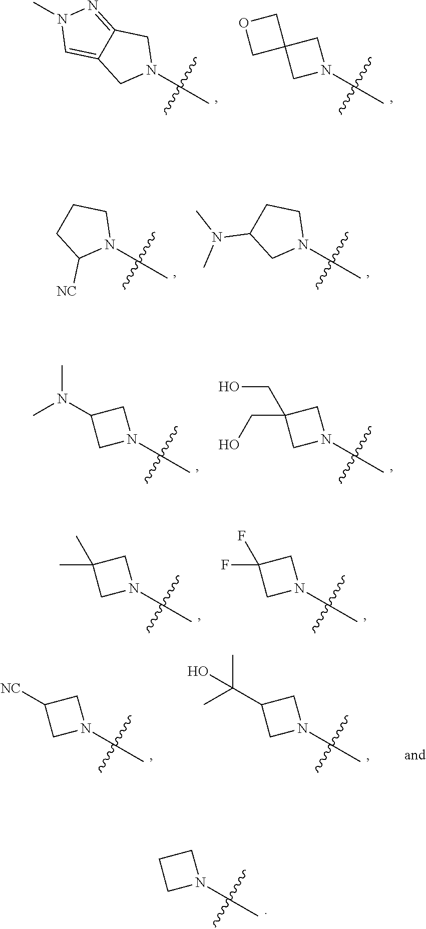

103. The method as recited in claim 102 wherein the nitrogen-containing heterocycloalkyl is chosen from:

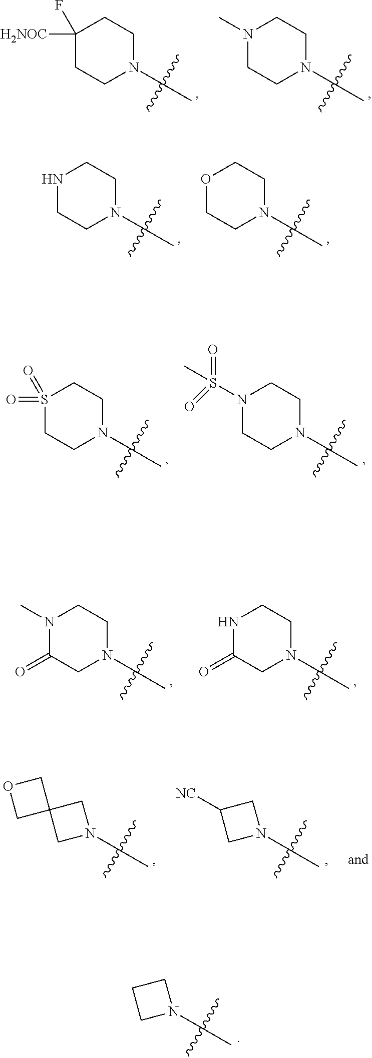

104. The method as recited in claim 103, wherein the nitrogen-containing heterocycloalkyl is:

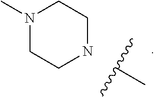

105. The method as recited in any of claims 91-104, wherein each R^{6a} is chosen from cyano, S(O)₂N(CH₃)₂,

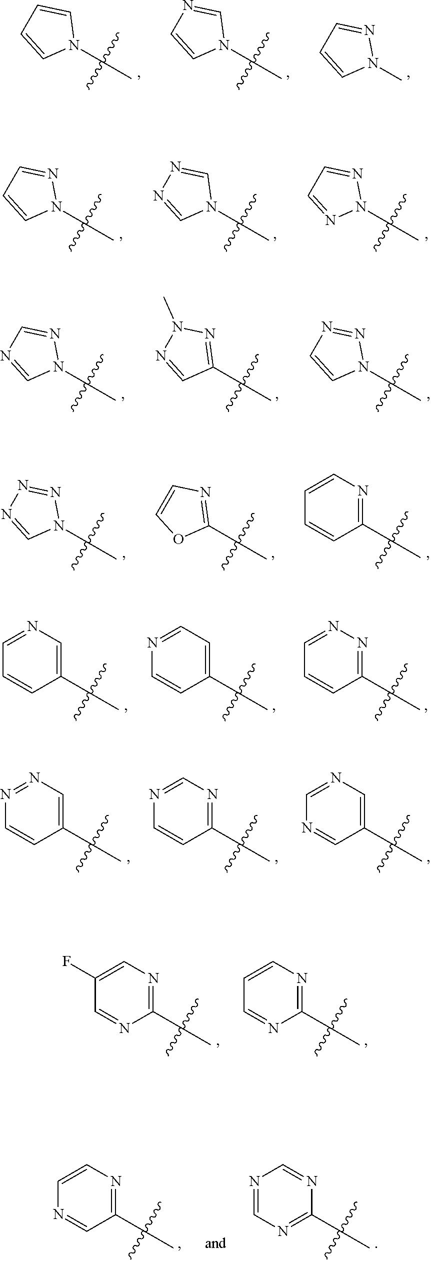

106. The method as recited in any of claims 91-105, wherein R⁵ is phenyl, which may be optionally substituted with between 0 and 3 R⁶ groups.

107. The method as recited in claim 95, wherein R⁵ is: 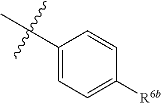
wherein R^{6b} is chosen from halogen, hydroxy, and methoxy.

108. The method as recited in claim 107, wherein R^{6b} is chosen from fluoro, methoxy, and hydroxy.

109. The method as recited in claim 108, wherein R^{6b} is fluoro.

110. The method as recited in claim 91, wherein the compound of Formula II is a compound of Formula III: 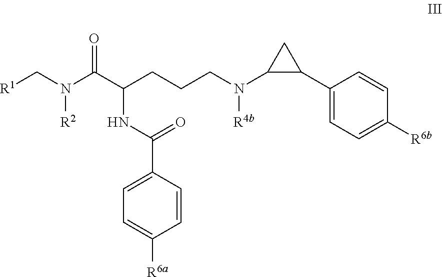
or a salt, polymorph, or solvate thereof, wherein:
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4a} is chosen from hydrogen, alkyl, alkenyl, alkynyl, and cycloalkyl;
R^{6b} is chosen from heteroaryl, cyano, and S(O)₂N(CH₃)₂;
each R⁶ and R^{6b} is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R′ and Re may be taken together to form a nitrogen-containing heterocycloalkyl or heteroaryl ring, which may be optionally substituted with lower alkyl;
with the proviso that when Y═CH₂ and Z═R^{4b}, then m+n≠3.

111. The method as recited in claim 110, wherein R^{4b} is chosen from methyl and hydrogen.

112. The method as recited in claim 111, wherein R^{4b} is hydrogen.

113. The method as recited in claim 112, wherein each R^{6a} is chosen from cyano,

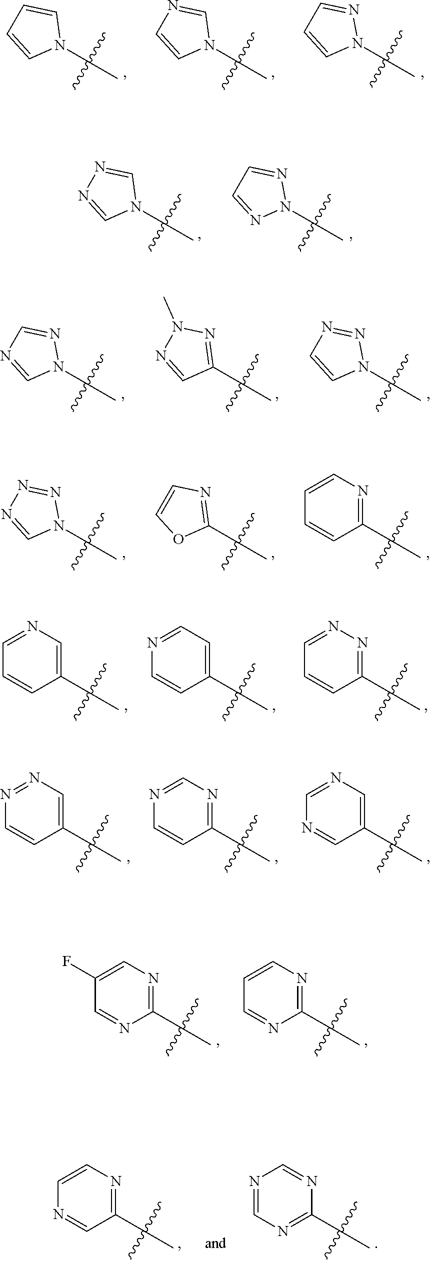

114. The method as recited in any of claims 110-113, wherein R¹ and R² are each independently chosen from alkyl, aminoalkyl, alkylsulfonylalkyl, alkoxyalkyl, and heteroaryl, and R¹ and R², together with the nitrogen to which they attach, form a nitrogen-containing heterocycloalkyl or heteroaryl ring, which may be optionally substituted with between 0 and 3 R⁶ groups.

115. The method as recited in claim 114, wherein the nitrogen-containing heterocycloalkyl or heteroaryl ring formed by R¹ and R² together with the nitrogen to which they are attached contains 3 to eight atoms.

116. The method as recited in claim 115, wherein R¹ and R² are taken together to form a nitrogen-containing heterocycloalkyl, which may be optionally substituted with between 0 and 3 R⁶ groups.

117. The method as recited in claim 116, wherein the nitrogen-containing heterocycloalkyl is chosen from:

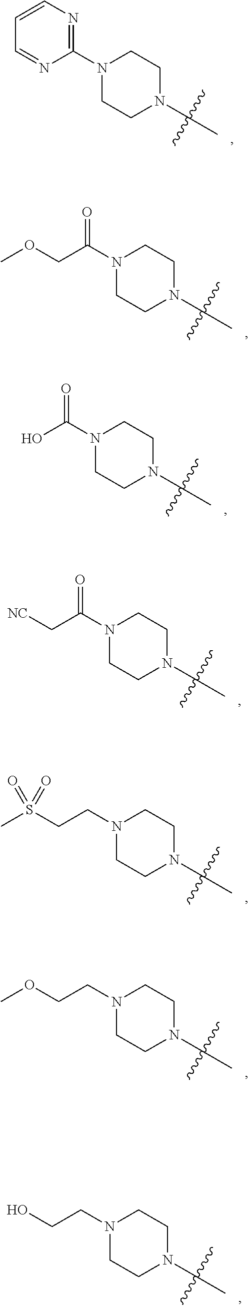

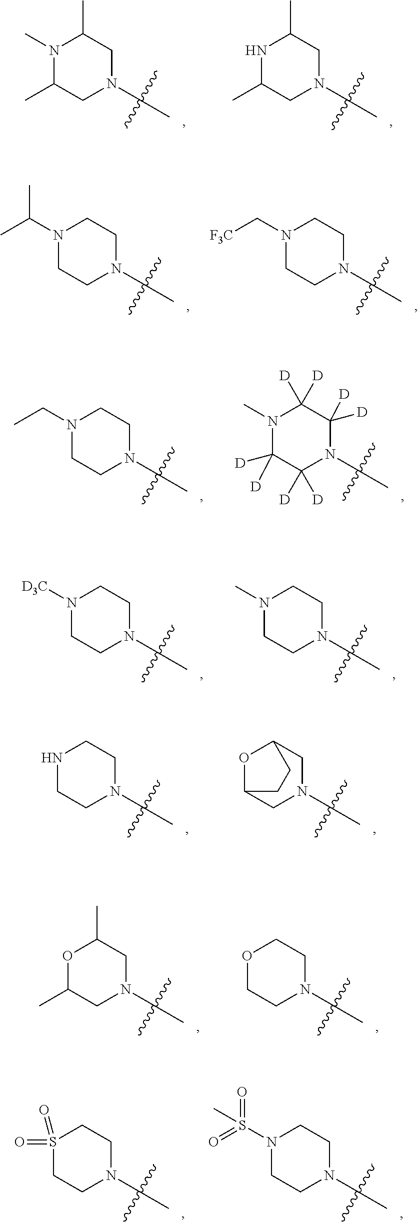

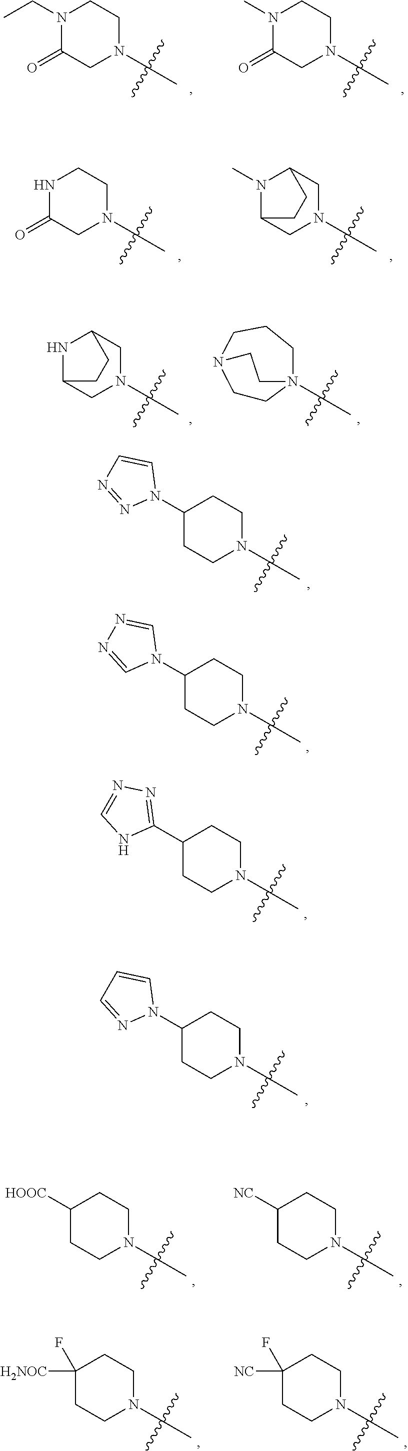

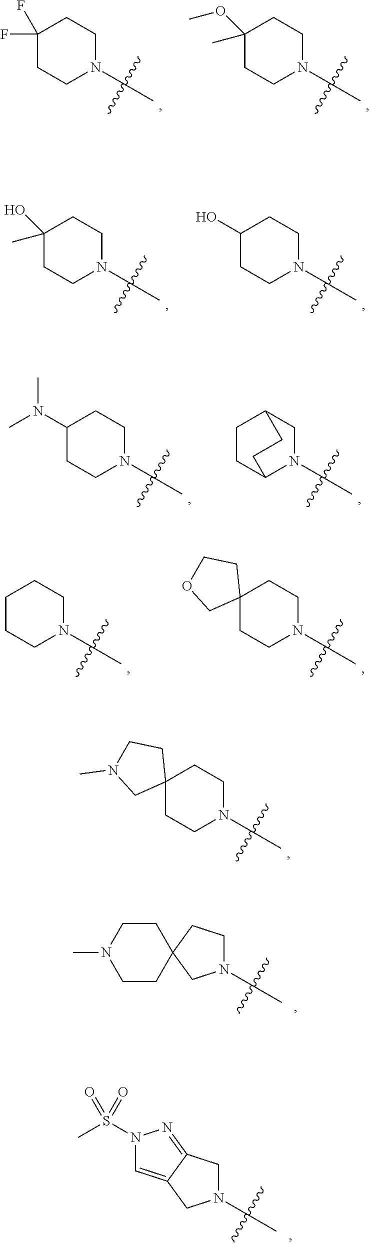

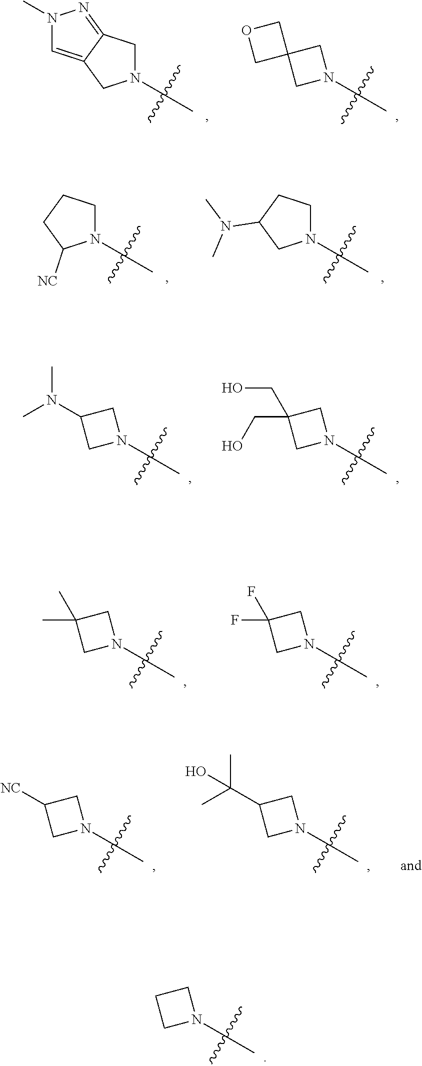

118. The method as recited in claim 117, wherein the nitrogen-containing heterocycloalkyl is chosen from:

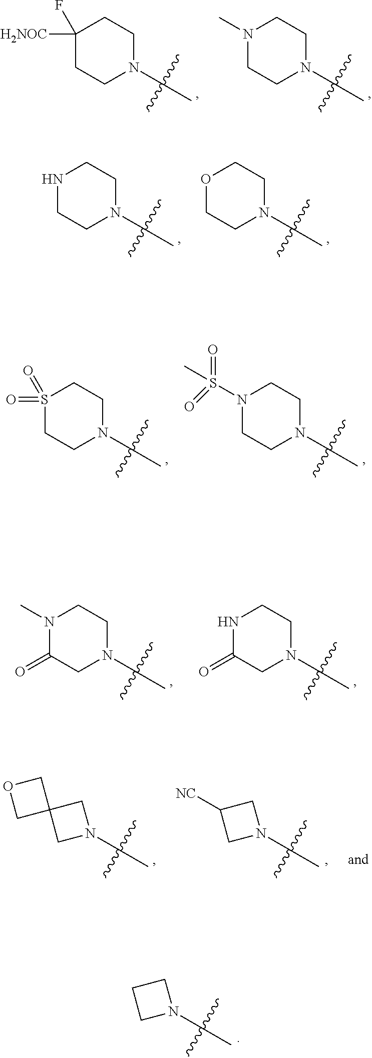

119. The method as recited in claim 118, wherein the nitrogen-containing heterocycloalkyl is:

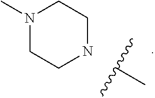

120. The method as recited in any of claims 110-119, wherein R^{6b} is chosen from fluoro, methoxy, and hydroxy.

121. The method as recited in claim 120, wherein R^{6b} is fluoro.

122. The method as recited in claim 110, wherein the compound of Formula III is: 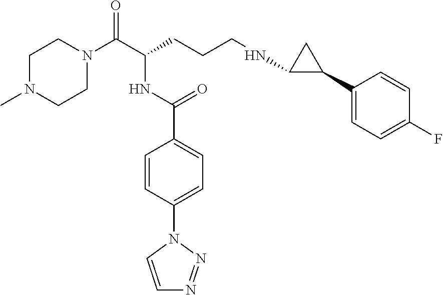 [SMILES: CN1CCN(C(=O)[C@H](CCCN[C@@H]2C[C@H]2c2ccc(F)cc2)NC(=O)c2ccc(-n3ccnn3)cc2)CC1]
or a salt, polymorph, or solvate thereof.

123. The method as recited in claim 122, wherein the compound is a salt of the formula: 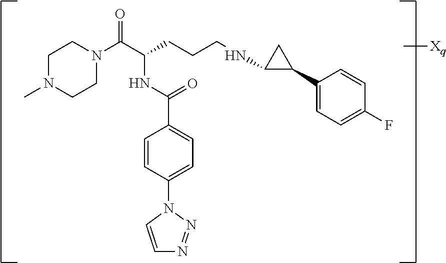
or a polymorph or solvate thereof, wherein:
X is chosen from tosylate, sulfate, tartrate, oxalate, besylate, fumarate, citric, esylate, and malate; and
q is an integer chosen from 1 and 2.

124. The method as recited in claim 71, wherein X is tosylate.

125. The method as recited in claim 72, wherein q is 2.

126. The method as recited in any one of claims 1-52 wherein the compound is chosen from amongst Examples A1-A34, Examples 1-188, and the Examples disclosed in Paragraph [0191] of the specification.

127. The method as recited in claim 126, wherein the compound is:

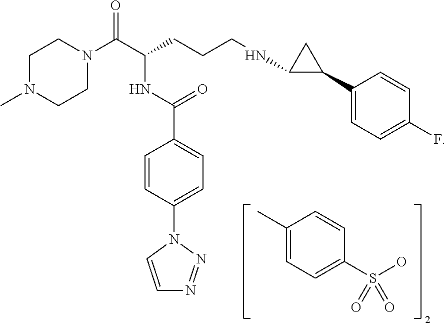

128. The method as recited in claim 126, wherein the compound is: 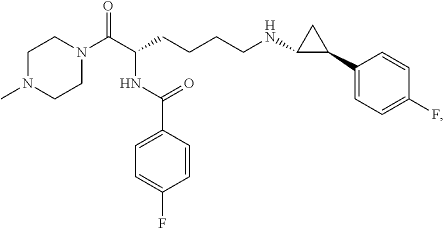 [SMILES: CN1CCN(C(=O)[C@H](CCCCN[C@@H]2C[C@H]2c2ccc(F)cc2)NC(=O)c2ccc(F)cc2)CC1]
or a salt thereof.
